(19) United States
(12) Patent Application Publication
(10) Pub. No.: US 2024/0058240 A1
(43) Pub. Date: Feb. 22, 2024

(54) COMPOUNDS AS SELF-TANNING SUBSTANCES AND TANNING COMPOSITIONS THEREOF

(71) Applicant: CHEMCOM S.A., Brussels (BE)

(72) Inventors: Sandra HUYSSEUNE, Bruxelles (BE); Alex VEITHEN, Genappe (BE); Yannick QUESNEL, Wavre (BE)

(21) Appl. No.: 18/003,303

(22) PCT Filed: Jun. 25, 2021
(86) PCT No.: PCT/EP2021/067466

§ 371 Date: Dec. 23, 2022

(30) Foreign Application Priority Data
Jun. 25, 2020 (EP) 20182311.9

(51) Int. Cl. A61K 8/34; A61K 8/33; A61K 8/35; A61K 8/37; A61K 8/49; A61K 8/63; A61Q 19/04; C07C 33/12

(52) U.S. Cl. CPC A61K 8/342; A61K 8/33; A61K 8/34; A61K 8/35; A61K 8/37; A61K 8/4973; A61K 8/498; A61K 8/63; A61Q 19/04; C07C 33/12; C07C 2602/44

(57) ABSTRACT

A self-tanning composition, such as a sunless tanning composition, a moisturizing composition, or a haircare composition, includes a self-tanning substance. The composition can result in increasing the amount of melanin in melanocytes, increasing melanin synthesis, improving melanin transport, and/or improving the distribution of melanin in suprabasal layers. The composition can be used in methods for darkening skin and/or for sunless tanning.

### PRIORITY AND CROSS REFERENCE TO RELATED APPLICATIONS

This application is a U.S. National Stage Application under 35 U.S.C. § 371 of International Application No. PCT/EP2021/067466, filed Jun. 25, 2021, designating the U.S. and published in English as WO 2021/260168 A1 on Dec. 30, 2021, which claims the benefit of European Patent Application No. EP 20182311.9, filed Jun. 25, 2020. Any and all applications for which a foreign or a domestic priority is claimed is/are identified in the Application Data Sheet filed herewith and is/are hereby incorporated by reference in their entirety under 37 C.F.R. § 1.57.

### REFERENCE TO ELECTRONIC SEQUENCE LISTING

The present application includes an Electronic Sequence Listing as an XML file submitted via EFS-Web. The Electronic Sequence Listing is provided as a file entitled DECLE242003APCSEQLIST.xml, created and last saved on Dec. 21, 2022, which is 8,530 bytes in size, which is replaced by a Replacement Electronic Sequence Listing submitted herewith as a file entitled DECLE242003APCREPLACEMENTSEQLIST.txt, which is 1,425 bytes in size and was created on Jul. 3, 2023. The information in the Electronic Sequence Listing is incorporated herein by reference in its entirety.

### FIELD OF INVENTION

The present invention relates to the use of organic compounds as self-tanning substances as well as tanning compositions comprising said organic compounds able to activate melanin synthesis, and the use thereof to induce an increase of melanin content in skin and thus inducing a tanning effect.

### BACKGROUND OF THE INVENTION

Melanins are predominantly indolic polymers that serve as the major pigment present in vertebrate skin, hair, fur and feathers. Melanin has many interesting properties among which its ability to absorb ultraviolet light (sunlight or other light sources) and prevent DNA damages. Two types of melanin co-exist: eumelanin-brown-black insoluble polymer- and pheomelanin-red-yellow soluble polymer. Eumelanin is the major type in individuals with dark skin/hair and is more efficient in photoprotection.

Skin has epidermal components that are responsible for melanin synthesis (melanocytes) and distribution (keratinocytes). Melanin biogenesis, called melanogenesis, occurs in melanosomes, a specialized lysosome-related organelle, present in melanocytes. This complex process includes different stages and starts from an oxidation of amino acid L-Tyrosine. Once synthesized, the melanin is transferred via cellular extensions (dendrites) of the melanocytes to the neighboring keratinocytes, the major cell type present in the epidermis (30-40 keratinocytes for 1 melanocyte).

Many factors are known as regulators of melanin synthesis but the most common is ultraviolet light exposure or Ultraviolet Radiation (UVR) that leads to sun-tanning, using either natural sunlight or ultraviolet light sources such as tanning lamps. The overall mechanism of tanning, i.e., production of melanin, is not fully understood, but at the melanocyte level, quite well described and accepted by the scientific community. Under UVRs stimulation, Melanocortin 1 receptor (MC1R), a G protein-couple receptor (GPCR), is expressed on the surface of melanocytes and regulates melanin synthesis qualitatively and quantitatively by increasing intracellular cyclic AMP (cAMP). MC1R function is regulated by the physiological agonists alpha Melanocyte Stimulating Hormone (αMSH), adrenocorticotropic hormone (ACTH) and by one antagonist agouti-signaling protein (ASP). Moreover, it has been demonstrated that people expressing less functional MC1R variants show a relative inability to tan (red-heads people) confirming the central role of this receptor and the cAMP cascade in melanogenesis.

Skin pigmentation is of great cultural, cosmetic importance and is considered as desirable by many persons. Nowadays, the most common way of darkening skin is sun-tanning using either natural sunlight or specially designed ultraviolet light sources (tanning lamps/beds). In the light of increasing incidence for UV-induced skin cancer/damages correlated with the progressive depletion of the ozone layer or excessive sunbath, other ways of tanning are of great interest. The first self-tanning lotion containing DiHydroxy Acetone (DHA) was put on the market in 1945 in California. Since 1960s, the popularity of sunless tanning grows in interest and chemists have spent tons of time to create self-tanner formulas that give a “natural brown” skin color. Almost all the currently available self-tanning products contain as active ingredient DHA, which may or may not be combined with erythrulose, tyrosine derivatives, and occasionally a naphthoquinone. DHA is a sugar surrogate that reacts with amino acids present in the skin trough the Maillard reaction, a well-known process to food chemists that causes the food browning during manufacturing, storage and cooking. Although DHA does not require UVs exposure to initiate color change, some studies reveal that DHA treated skins are more susceptible to UVs and generate a huge amount of additional free radicals inside. Moreover, most sunless tanning products do not contain sunscreen. If a sunscreen product is present, it will only be effective for a couple of hours. Self-tanning does not confer any significant UV protection. Besides this lack of protective effect, DHA has a so called “crust-like” odor once applied on the skin. The odor issue varies from person to person depending on their body chemistry.

Therefore, there is a need for the correct regulation of melanogenesis as it is an extremely important aspect of skin characteristics and constitutes a response to the environmental stresses. So far, beside exposition to UV, there are few if any reliable methods that stimulate the production of melanin in the skin to allow a natural tanning and a significant UV protection.

The present invention addresses these needs.

### SUMMARY OF THE INVENTION

The present invention provides tanning compositions comprising tanning compounds that can induce self-tanning in a safe manner while avoiding the development of undesirable odors.

In particular, in a first aspect, the present invention provides a tanning composition comprising:
a) a cosmetically acceptable carrier, diluent and/or excipient and
b) at least one tanning compound selected from the group comprising a compound of formula (I), (II), (III) and (IV), or a racemate, a stereoisomer, a tautomer, a salt, and/or a solvate thereof, including mixtures thereof,

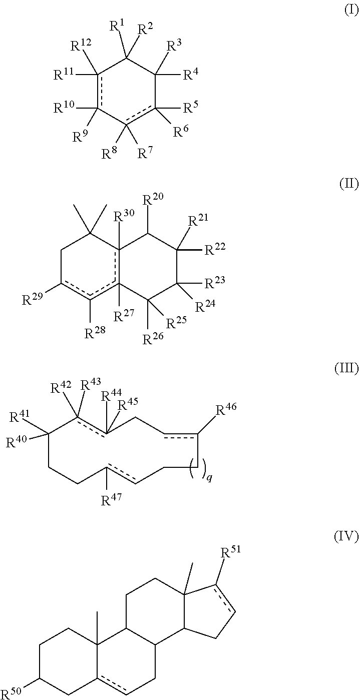

wherein R¹, R², R³, R⁴, R⁵, R⁶, R⁷, R⁸, R⁹, R¹⁰, R¹¹, R¹², R²⁰, R²¹, R²², R²³, R²⁴, R²⁵, R²⁶, R²⁷, R²⁸, R²⁹, R³⁰, R⁴⁰, R⁴¹, R⁴², R⁴³, R⁴⁴, R⁴⁵, R⁴⁶, R⁴⁷, q, R⁵⁰, R⁵¹ are as defined herein.

In a preferred aspect, the present invention provides the use of a compound of formula (IIb), (IIa), (IIIa), (IV), (Ie), (If), (Ig), (Ic) or (Id), a racemate, a stereoisomer, a tautomer, a salt, and/or a solvate thereof, including mixtures thereof, as self-tanning substance,

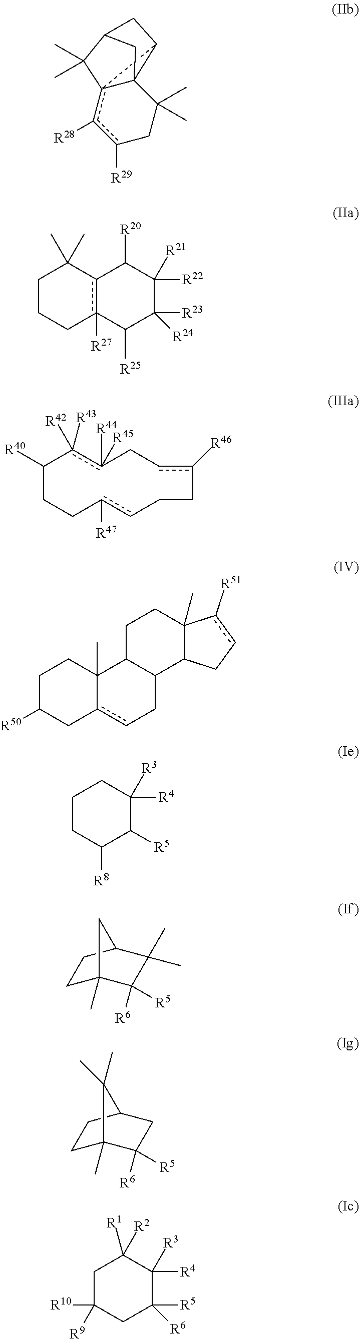

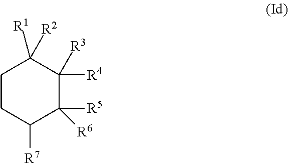

wherein the dash bond [symbol] represents an optional double bond; and wherein [symbol] represent an optional single bond;
R²⁸ is selected from the group comprising hydrogen, oxo, C₁₋₆alkyl, —O—C₁₋₆alkyl, —[CR¹⁷R¹⁸]ₘ—O—CO—C₁₋₆alkyl, —[CR¹⁷R¹⁸]ₘ—O—C₁₋₆alkyl, —[CR¹⁷R¹⁸]ₘ—COH, and —[CR¹⁷R¹⁸]ₘ—OH; m is an integer selected from 1, 2, 3, 4 or 5,
R¹⁷, R¹⁸, are each independently selected from hydrogen or C₁₋₆alkyl;
R²⁹ is hydrogen or C₁₋₆alkyl;
or R²⁹ and R²⁸ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2;
R²⁰ is hydrogen or C₁₋₆alkyl;
R²¹ is hydrogen or C₁₋₆alkyl;
R²² is selected from the group comprising hydrogen, C₁₋₆alkyl, and —CO—R¹⁹;
R²³ is hydrogen or C₁₋₆alkyl;
R²⁴ is hydrogen or C₁₋₆alkyl;
or R²³ and R²⁴ together with the carbon atom to which they are attached form a 3- to 6-membered heterocyclyl moiety; wherein said heterocyclyl is optionally substituted with one or more substituents each independently selected from C₁₋₆alkyl;
R²⁵ is hydrogen or C₁₋₆alkyl;
or R²⁴ and R²⁵ form together with the carbon atom to which they are attached a 5 or 6-membered heterocyclyl moiety, wherein said heterocyclyl is optionally substituted with one or more substituents each independently selected from C₁₋₆alkyl, or wherein one of said substituent and R²³ form together with the atoms to which they are attached a 3- to 6-membered heterocyclyl moiety;
R²⁷ if present is hydrogen or C₁₋₆alkyl;
or R²⁰ and R²⁷ together with the carbon atoms to which they are attached form a C3-membered ring;
R⁴⁰ is selected from the group comprising —[CR¹⁷R¹⁸]ₘ—OH, —CO—C₁₋₆alkyl, —O—([CR¹⁷R¹⁸]ₘ—O—)ₜ—C₁₋₆ alkyl, t is an integer selected from 0, 1, 2, or 3;
R⁴², R⁴³, R⁴⁴, R⁴⁵, R⁴⁶, R⁴⁷ are each independently selected from hydrogen or C₁₋₆alkyl;
or R⁴⁰ and R⁴² together with the carbon atom to which they are attached form a 3- to 6-membered heterocyclyl moiety;
R⁵⁰ is selected from OH or oxo;
R⁵¹ is hydrogen or C₁₋₄alkyl;
R¹, R² are each independently selected from hydrogen or C₁₋₆alkyl;
R³, R⁴ are each independently selected from hydrogen or C₁₋₆alkyl;
or R¹ with R³ together with the carbon atoms to which they are attached form a saturated C3 to C5-membered ring, wherein said ring is optionally substituted with one or more C₁₋₆alkyl substituents;
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —X—[CR¹³R¹⁴]ₙ—O—[CR¹⁷R¹⁸]ₘ—OH, —([CR¹⁷R¹⁸]ₘ—O)_{y}—C₁₋₆alkyl, —[CR¹³R¹⁴]ₙ—CO—R¹⁹;
m is an integer selected from 1, 2, 3, 4 or 5, n is an integer selected from 0, 1, 2, 3 or 4; y is an integer selected from 1, 2 or 3;
X is a single bond or O,
R¹³, R¹⁴, R¹⁶, R¹⁷, R¹⁸, R¹⁹ are each independently selected from hydrogen or C₁₋₆alkyl;
R¹⁵ is OH, or R¹⁵ together with R¹⁶ form an oxo group;
or R³ and R⁵ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2;
R⁶ is hydrogen or C₁₋₆alkyl;
or R⁵ and R⁶ form together ═[CR¹⁴]—[CR¹⁷R¹⁸]ₘ—OH; or R⁵ and R⁶ together with the carbon atom to which they are attached form a 5- or 6-membered heterocyclyl moiety, wherein said heterocyclyl is optionally substituted with one or more C₁₋₆alkyl substituents;
R⁷ is hydrogen or C₁₋₆alkyl;
R⁸ is hydrogen or C₁₋₆alkyl;
or R⁵ and R⁸ together with the carbon atoms to which they are attached form a saturated or unsaturated C5 or C6-membered ring, wherein said ring is optionally substituted with one or more substituents each independently selected from oxo, C₁₋₆alkyl, —O—C₁₋₆alkyl, —CO—C₁₋₆alkyl, —[CR¹⁷R¹⁸]ₘ—O—CO—C₁₋₆alkyl, —([CR¹⁷R¹⁸]ₘ—O)_{y}—C₁₋₆alkyl, —[CR¹⁷R¹⁸]ₘ—COH, or —[CR¹⁷R¹⁸]ₘ—OH; or two substituents on said ring form together with the carbon atoms to which they are attached a 3- to 6-membered heterocyclyl moiety, wherein said heterocyclyl moiety is optionally substituted with one or more substituents each independently selected from C₁₋₆alkyl, or two substituents on said heterocyclyl form together with the atoms to which they are attached a 3- to 6-membered heterocyclyl moiety;
R⁹, R¹⁰ are each independently selected from hydrogen or C₁₋₆alkyl;
or R⁶ and R⁹ together with the carbon atoms to which they are attached form a C3-membered ring;
or R¹ with R⁹ together with the carbon atoms to which they are attached form a saturated C5-membered ring, wherein said ring is optionally substituted with one or more C₁₋₆alkyl substituents;
or R³ with R⁹ together with the carbon atoms to which they are attached form a saturated C5-membered ring, wherein said ring is optionally substituted with one or more C₁₋₆alkyl substituents.

The present invention also encompasses self-tanning compositions prepared using at least one compound of formula (IIb), (IIa), (IIIa), (IV), (Ie), (If), (Ig), (Ic), or (Id), a racemate, a stereoisomer, a tautomer, a salt, and/or a solvate thereof, including mixtures thereof, as self-tanning substances.

The present invention also provides, in a second aspect, a compound of formula (Ih), or a racemate, a stereoisomer, a tautomer, a salt, and/or a solvate thereof, including mixtures thereof;

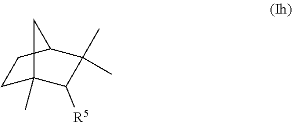

wherein
R⁵ is selected from the group comprising —O—CHR¹⁴—C(═O)—[CR¹⁷R¹⁸]ₘ—R¹⁹, —O—CHR¹⁴—CH(OH)—[CR¹⁷R¹⁸]ₘ—R¹⁹;
m is an integer selected from 1, 2, 3, 4 or 5; preferably m is selected from 1, 2, 3 or 4, preferably m is selected from 1, 2, or 3, preferably m is 1 or 2;
R¹⁴, R¹⁷, R¹⁸, R¹⁹ are each independently selected from hydrogen or C₁₋₆alkyl; preferably R¹⁴, R¹⁷, R¹⁸, R¹⁹ are each independently selected from hydrogen or C₁₋₄ alkyl; preferably R¹⁴, R¹⁷, R¹⁸, R¹⁹ are each independently selected from hydrogen or C₁₋₂alkyl; preferably R¹⁴, R¹⁷, R¹⁸, R¹⁹ are each independently selected from hydrogen or methyl; preferably R¹⁴, R¹⁷, R¹⁸, are each independently hydrogen and R¹⁹ is methyl.

The present invention also provides, in a third aspect, the use of a compound of formula (I), (II), (III) or (IV) as defined herein, or a racemate, a stereoisomer, a tautomer, a salt, and/or a solvate thereof, including mixtures thereof, as self-tanning substance; or the use of a compound according to the second aspect of the invention as self-tanning substance;

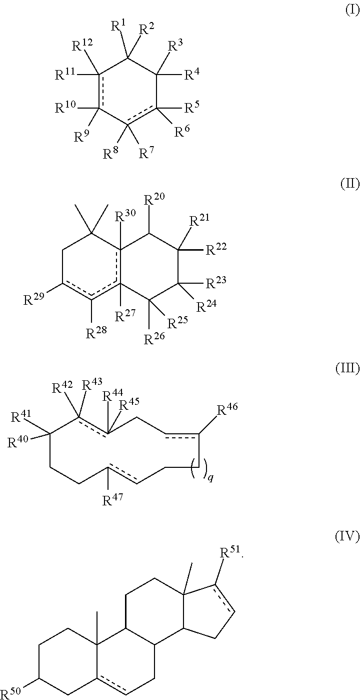

The present invention also provides, in a fourth aspect, a compound of formula (I), (II), (III), (IV), (IIb), (IIa), (IIIa), (Ie), (If), (Ig), (Ic), or (Id) as defined herein, or a racemate, a stereoisomer, a tautomer, a salt, and/or a solvate thereof, including mixtures thereof, for use for increasing the amount of melanin in melanocytes, for increasing melanin synthesis, for improving melanin transport and/or for improving the distribution of melanin in suprabasal layers; or the compound according to the second aspect of the invention for use for increasing the amount of melanin in melanocytes, for increasing melanin synthesis, for improving melanin transport and/or for improving the distribution of melanin in suprabasal layers;

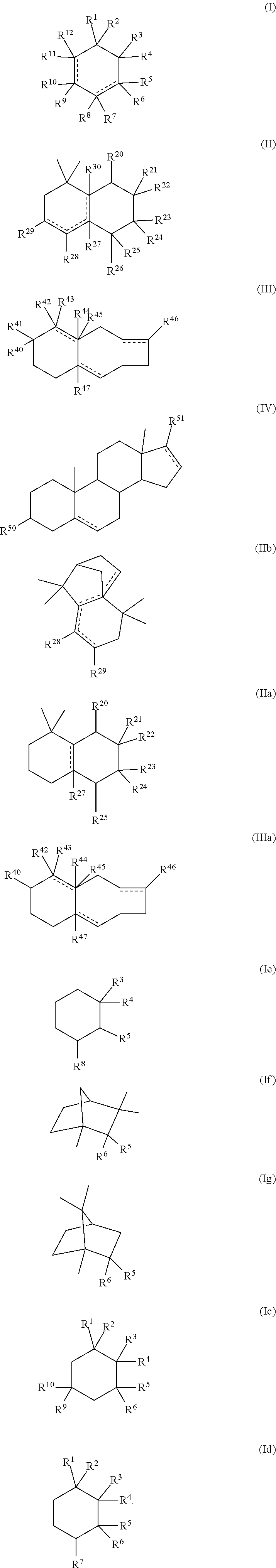

The present invention also encompasses the use of a composition according to the first aspect for darkening skin, or the preferred use of a compound of formula (IIb), (IIa), (IIIa), (IV), (Ie), (If), (Ig), (Ic), or (Id) for darkening skin, or the use of a compound of formula (I), (II), (III), (IV) or (Ih) according to the second or third aspect, for darkening skin.

The present invention also encompasses the use of a composition according to the first aspect for sunless tanning, or the preferred use of a compound of formula (IIb), (IIa), (IIIa), (IV), (Ie), (If), (Ig), (Ic), or (Id) for sunless tanning, or the use of a compound of formula (I), (II), (III), (IV) or (Ih) according to the second or third aspect, for sunless tanning.

The present invention also encompasses a tanning method comprising using a composition according to the first aspect, or a compound according to the second aspect as a self-tanning substance, or the use of a compound of formula (I), (II), (III) or (IV) according to the third aspect as a self-tanning substance, or the preferred use of a compound of formula (IIb), (IIa), (IIIa), (IV), (Ie), (If), (Ig), (Ic), or (Id) as a self-tanning substance.

The present invention also encompasses a method for increasing the amount of melanin in melanocytes, for increasing melanin synthesis, for improving melanin transport and/or for improving the distribution of melanin in suprabasal layers comprising applying a composition according to the first aspect, or a compound according to the second aspect, or applying one of more compounds of the formula (I), (II), (III), (IV) or (Ih), or any subgroup thereof such as a compound of formula (IIb), (IIa), (IIIa), (Ie), (If), (Ig), (Ic), or (Id), as defined herein.

The present invention also encompasses a method of darkening skin comprising the step of applying a composition according to according to the first aspect, or a compound according to the second aspect, or applying one of more compounds of the formula (I), (II), (III), (IV), (IIb), (IIa), (IIIa), (Ie), (If), (Ig), (Ic), or (Id) or a racemate, a stereoisomer, a tautomer, a salt, and/or a solvate thereof, including mixtures thereof, as defined herein, onto a desired skin surface.

In the present invention, it has surprisingly been discovered that the olfactory receptor OR7C1 is expressed in melanocytes and that agonists of this receptor can trigger the production of melanin by melanocytes, leading to an increase of their melanin content that can therefore increase pigmentation. Therefore, the present invention also encompasses the use of agonists of human olfactory receptor family seven, subfamily C, member one; i.e., OR7C1, particularly those expressed on melanocytes, for increasing the amount of melanin in melanocytes, for increasing melanin synthesis, for improving melanin transport and/or for improving the distribution of melanin in suprabasal layers and thus inducing a tanning effect. The present invention also encompasses the use of agonists of human olfactory receptor family seven, subfamily C, member one; i.e., OR7C1, particularly those expressed on melanocytes, as a self-tanning substance. The present invention also encompasses a tanning composition comprising (a) a cosmetically acceptable carrier, diluent and/or excipient and (b) at least one tanning compound selected from the group comprising agonists of human olfactory receptor family seven, subfamily C, member one; i.e., OR7C1, particularly those expressed on melanocytes.

The compounds for use in the present invention can induce melanin synthesis in melanocytes and thereby increasing the melanin content, and thus subject pigmentation such as skin, hair, fur and feathers pigmentation by the compounds defined herein is also encompassed herein. The melanocytes that are in contact with the compounds of invention may be present in vertebrate skin, hair, fur, and feathers.

The compounds for use in the present invention can be used as skin tanning agents. A melanin overproduction in the skin can therefore be induced, thereby preventing sunburns, improving UVR's skin protection or increasing skin pigmentation, for example in a non-therapeutic manner. Given the importance of UV-induced skin cancer/damages in the future, this unexpected discovery would allow a safer way of tanning and avoid UVRs deleterious effects by activating the endogenous and natural tanning pathway.

The above and other characteristics, features, and advantages of the present invention will become apparent from the following detailed description, which illustrate, by way of example, the principles of the invention.

### BRIEF DESCRIPTION OF THE DRAWINGS

FIG. 1 represents a schematic representation of the treatment of Normal Human Epidermal Melanocytes-Moderately Pigmented (NHEMs-MP) with 2 different compounds according to embodiments of the invention (Amberketal and Ambermax) to test the effects of said compounds on cell viability.

FIG. 2 represents a schematic representation of the treatment of NHEMs-MP with 2 different compounds according to embodiments of the invention (Amberketal and Ambermax) to test the effect of these compounds on the pigmentation of NHEMs-MP in culture.

FIG. 3 represents schematic representations of explants treatment with 2 different compounds according to embodiments of the invention to test the tanning effect.

FIG. 4A represents photographs showing the results of PCR experiment on cDNA prepared after mRNA extraction of primary melanocytes (NHEMs-MP). Results are shown as a cDNA amplification in an agarose gel after PCR using 3 different couples of primers (couple 1, 2 and 3, see experimental procedures).

FIG. 4B represents a graph illustrating the effects of 2 compounds according to embodiments of the invention on the survival of primary melanocytes in culture (MTS test, see experimental procedures). Results are expressed as average of relative viability values +/−SEM of 3 measurements; n=3.

FIG. 5A represents a graph representing the effects of 2 compounds according to embodiments of the invention (Ambermax and Amberketal) on intracellular melanin production in melanocytes (NHEMs-MP). Results are represented as average of relative values of the melanin content in the DOPA (50 μM) condition +/−SEM of 12 measurements; n=12.

FIG. 5B represents a graph representing the effects of 2 compounds according to embodiments of the invention (Ambermax and Amberketal) on extracellular melanin production in melanocytes (NHEMs-MP). Results are represented as relative values of the melanin content in the DOPA (50 μM) condition +/−SEM of 10 measurements; n=10.

FIG. 6A shows representative pictures of explants stained with the Trichrome Masson, Goldner variant method, to assess the effect of compound according to embodiments of the invention on the survival of human skin living explants. Biopsies were treated topically with 10 μM, 30 μM and 100 μM of archetypal compound of invention at day 5 (D5 or J5). T corresponds to untreated explants (negative controls).

FIG. 6B represents a table showing the summary of the macroscopic assessment of the survival of human skin living explants after treatments with compound according to embodiments of the invention at 3 concentrations and with 3 ways of administration (topical, systemic, and intradermal injections) at day 5 (D5 or J5) and day 10 (D10 or J10). T corresponds to untreated explants (negative controls), TUV are explants irradiated with a UVA dose of 0.5 MED (positive controls).

FIG. 6C represents a table with photographs of skin explants showing the level of melanin production due to topical treatments (10 μM, 30 μM and 100 μM) with compounds according to embodiments of the invention on epidermal living explants of human epidermis after 5 days (Masson's Fontana staining method).

FIG. 6D represents graph plotting the melanin levels as a function of time, showing the level of melanin production due to topical treatments (10 μM, 30 μM and 100 μM) with compounds according to embodiments of the invention on epidermal living explants of human epidermis after 5 and 10 days (Masson's Fontana staining method).

FIG. 6E represents a table summarizing the macroscopic assessment of the level of melanin present in human skin living explants after treatments with compounds according to embodiments of the invention at 3 concentrations and with 3 ways of administration (topical, systemic and intradermal injections) at D5 and D10. T corresponds to untreated explants (negative controls), TUV are explants irradiated with a UVA dose of 0.5 MED (positive controls). Results are expressed as melanin content present at day 5 or 10 in comparison with melanin content present in non-exposed explant or UV exposed explant at the same time frame.

FIG. 6F represents a table with photographs of skin explants showing an increase of the level of melanin due to topical treatment (0.3 μM, 1 μM, 10 and 15 μM) with compound according to embodiments of the invention on epidermal living explants of human epidermis after 6 days (Masson's Fontana staining method).

FIG. 6G represents a graph plotting the percentage of surface positive to melanin staining in the assessment of the level of melanin production due to topical treatment (1 μM, 3 μM, 10 and 15 μM) with compound according to embodiments of the invention on epidermal living explants of human epidermis after 6 days (Masson's Fontana staining method). Results are average +/−SEM of single measurement on 3 different skin explants.

FIG. 6H represents a table summarizing the effect of compounds according to embodiments of the invention on melanin content in living explants after topical treatment with archetypal compound of invention (0.3, 1, 10 and 15 μM). T corresponds to untreated explants (negative controls), TUV are explants irradiated with a UVA dose of 0.5 MED (positive controls). Results are expressed as melanin content present at day 6 or 10 in comparison with melanin content present in non-exposed explant or UV exposed explant at the same time frame.

FIGS. 7A to 7D represent schematic representations of an image to be analyzed.

### DETAILED DESCRIPTION OF THE INVENTION

When describing the invention, the terms used are to be construed in accordance with the following definitions, unless a context dictates otherwise.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention, and form different embodiments, as would be understood by those in the art.

The terms “comprising”, “comprises” and “comprised of” as used herein are synonymous with “including”, “includes” or “containing”, “contains”, and are inclusive or open-ended and do not exclude additional, non-recited members, elements, or method steps. It will be appreciated that the terms “comprising”, “comprises” and “comprised of” as used herein comprise the terms “consisting of”, “consists” and “consists of”.

As used in the specification and the appended claims, the singular forms “a”, “an,” and “the” include plural referents unless the context clearly dictates otherwise. By way of example, “a step” means one step or more than one step.

Unless defined otherwise, all technical and scientific terms used herein have the same meaning as is commonly understood by one of skill in the art. All publications referenced herein are incorporated by reference thereto.

The recitation of numerical ranges by endpoints includes all integer numbers and, where appropriate, fractions subsumed within that range (e.g., 1 to 5 can include 1, 2, 3, 4 when referring to, for example, a number of elements, and can also include 1.5, 2, 2.75 and 3.80, when referring to, for example, measurements). The recitation of end points also includes the end point values themselves (e.g., from 1.0 to 5.0 includes both 1.0 and 5.0). Any numerical range recited herein is intended to include all sub-ranges subsumed therein.

Whenever the term “substituted” is used in the present invention, it is meant to indicate that one or more hydrogens on the atom indicated in the expression using “substituted” is replaced with a selection from the indicated group, provided that the indicated atom's normal valency is not exceeded, and that the substitution results in a chemically stable compound. Where groups can be substituted, such groups may be substituted with one or more, and preferably one, two or three substituents.

The term “alkyl”, as a group or part of a group, refers to a hydrocarbyl group of formula CₙH₂ₙ₊₁ wherein n is a number of at least 1. Alkyl groups may be linear or branched and may be substituted as indicated herein. Preferably, the alkyl group comprises from 1 to 6 carbon atoms, preferably from 1 to 5 carbon atoms, preferably from 1 to 4 carbon atoms, preferably from 1 to 3 carbon atoms, preferably from 1 to 2 carbon atoms. When a subscript is used herein following a carbon atom, the subscript refers to the number of carbon atoms that the named group may contain. For example, the term “C₁₋₆alkyl”, as a group or part of a group, refers to a hydrocarbyl group of Formula CₙH₂ₙ₊₁ wherein n is a number ranging from 1 to 6. For example, C₁₋₆alkyl includes all linear or branched alkyl groups having 1 to 6 carbon atoms, and thus includes for example methyl, ethyl, n-propyl, i-propyl, 2-methyl-ethyl, butyl, and its isomers (e.g., n-butyl, i-butyl, and t-butyl); pentyl and its isomers, hexyl and its isomers, and the like. For example, C₁₋₄alkyl includes all linear or branched alkyl groups having 1 to 4 carbon atoms, and thus includes for example methyl, ethyl, n-propyl, i-propyl, 2-methyl-ethyl, butyl, and its isomers (e.g., n-butyl, i-butyl, and t-butyl), and the like.

The term “oxo” refers to the group ═O.

The term “heterocyclyl moiety” or “heterocyclyl ring” as used herein refer to non-aromatic, fully saturated or partially unsaturated ring of 3 to 6 atoms including at least one N, O, or S. The N and S heteroatoms may optionally be oxidized, and the N heteroatoms may optionally be quaternized, and wherein at least one carbon atom of heterocyclyl can be oxidized to form at least one C═O.

The heterocyclic may be attached at any heteroatom or carbon atom of the ring or ring system, where valence allows.

Non limiting exemplary heterocyclic groups include oxiranyl, piperidinyl, piperazinyl, morpholinyl, tetrahydropyranyl, tetrahydrofuranyl, pyrrolidinyl, aziridinyl, thiiranyl, azetidinyl, oxetanyl, thietanyl, imidazolinyl, pyrazolidinyl imidazolidinyl, isoxazolinyl, oxazolidinyl, isoxazolidinyl, thiazolidinyl, isothiazolidinyl, succinimidyl, 2H-pyrrolyl, 1-pyrrolinyl, 2-pyrrolinyl, 3-pyrrolinyl, 2-pyrazolinyl, 3-pyrazolinyl, tetrahydro-2H-pyranyl, 2H-pyranyl, 4H-pyranyl, 3,4-dihydro-2H-pyranyl, 1,3-dioxolanyl, 1,3-dioxanyl, 1,4-dioxanyl, tetrahydrothiophenyl, thiomorpholin-4-yl, 1,4-oxathianyl, 1,4-dithianyl, and 1,3,5-trioxanyl. Preferably heterocyclyl is selected from the group comprising oxiranyl, tetrahydropyranyl, tetrahydrofuranyl, oxetanyl, 2H-pyrrolyl, 1-pyrrolinyl, 2-pyrrolinyl, 3-pyrrolinyl, tetrahydro-2H-pyranyl, 2H-pyranyl, 4H-pyranyl, 3,4-dihydro-2H-pyranyl, 1,3-dioxolanyl, 1,3-dioxanyl, and 1,4-dioxanyl.

The term “single bond” as used herein for a linking group i.e., in a way that a certain linking group is selected from a single bond, etc. in the formulas herein, refers to a molecule wherein the linking group is not present and therefore refers to compounds with a direct linkage via a single bond between the two moieties being linked by the linking group.

As used herein and unless otherwise stated, the term “solvate” includes any combination which may be formed by a derivative of this invention with a suitable inorganic solvent (e.g., hydrates) or organic solvent, such as but not limited to alcohols, ketones, esters, ethers, nitriles and the like.

The term “isomers” as used herein means all possible isomeric forms, including tautomeric and stereochemical forms, which the compounds of formulae herein may possess, but not including position isomers. Typically, the structures shown herein exemplify only one tautomeric or resonance form of the compounds, but the corresponding alternative configurations are contemplated as well. Unless otherwise stated, the chemical designation of compounds denotes the mixture of all possible stereochemically isomeric forms, said mixtures containing all diastereomers and enantiomers (since the compounds of formulae herein may have at least one chiral center) of the basic molecular structure, as well as the stereochemically pure or enriched compounds. More particularly, stereogenic centers may have either the R- or S-configuration, and multiple bonds may have either cis- or trans-configuration.

Pure isomeric forms of the said compounds are defined as isomers substantially free of other enantiomeric or diastereomeric forms of the same basic molecular structure. In particular, the term “stereoisomerically pure” or “chirally pure” relates to compounds having a stereoisomeric excess of at least about 80% (e.g., at least 90% of one isomer and at most 10% of the other possible isomers), preferably at least 90%, more preferably at least 94% and most preferably at least 97%. The terms “enantiomerically pure” and “diastereomerically pure” should be understood in a similar way, having regard to the enantiomeric excess, respectively the diastereomeric excess, of the mixture in question.

Separation of stereoisomers is accomplished by standard methods known to those in the art. One enantiomer of a compound of the invention can be separated substantially free of its opposing enantiomer by a method such as formation of diastereomers using optically active resolving agents (“Stereochemistry of Carbon Compounds,” (1962) by E. L. Eliel, McGraw Hill; Lochmuller, C. H., (1975) J. Chromatogr., 113:(3) 283-302). Separation of isomers in a mixture can be accomplished by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure enantiomers, or (3) enantiomers can be separated directly under chiral conditions. Under method (1), diastereomeric salts can be formed by reaction of enantiomerically pure chiral bases such as brucine, quinine, ephedrine, strychnine, a-methyl-b-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts. Alternatively, by method (2), the substrate to be resolved may be reacted with one enantiomer of a chiral compound to form a diastereomeric pair (Eliel, E. and Wilen, S. (1994) Stereochemistry of Organic Compounds, John Wiley & Sons, Inc., p. 322). Diastereomeric compounds can be formed by reacting asymmetric compounds with enantiomerically pure chiral derivatizing reagents, such as menthyl derivatives, followed by separation of the diastereomers and hydrolysis to yield the free, enantiomerically enriched compound. A method of determining optical purity involves making chiral esters, such as a menthyl ester or Mosher ester, a-methoxy-a-(trifluoromethyl)phenyl acetate (Jacob III. (1982) J. Org. Chem. 47:4165), of the racemic mixture, and analyzing the NMR spectrum for the presence of the two atropisomeric diastereomers. Stable diastereomers can be separated and isolated by normal- and reverse-phase chromatography following methods for separation of atropisomeric naphthyl-isoquinolines (Hoye, T., WO 96/15111). Under method (3), a racemic mixture of two asymmetric enantiomers is separated by chromatography using a chiral stationary phase. Suitable chiral stationary phases are, for example, polysaccharides, in particular cellulose or amylose derivatives. Commercially available polysaccharide based chiral stationary phases are ChiralCel™ CA, OA, OB5, OC5, OD, OF, OG, OJ and OK, and Chiralpak™ AD, AS, OP(+) and OT(+). Appropriate eluents or mobile phases for use in combination with said polysaccharide chiral stationary phases are hexane and the like, modified with an alcohol such as ethanol, isopropanol, and the like. (“Chiral Liquid Chromatography” (1989) W. J. Lough, Ed. Chapman and Hall, New York; Okamoto, (1990) “Optical resolution of dihydropyridine enantiomers by High-performance liquid chromatography using phenylcarbamates of polysaccharides as a chiral stationary phase”, J. of Chromatogr. 513:375-378).

As used herein, the term “Olfactory Receptor polypeptides (ORs)” in general refers to polypeptides from the G protein coupled receptor family mainly expressed by olfactory neurons. ORs may have the ability to interact with odorant molecules and to transduce cAMP cascade.

As used herein, the term “G-Protein coupled receptor,” or “GPCR” refers to a membrane-associated polypeptide with 7 alpha helical transmembrane domains. Functional GPCRs associate with a ligand or agonist and also associate with and activate G-proteins. The OR7C1 as defined herein belongs to the family of GPCRs.

As used herein, the term “OR binding” refers to specific binding of an odorant molecule by an OR polypeptide. Examples of odorant molecules include but are not limited to compounds from the formulation described below (Markush).

As used herein, the term “OR signaling activity” refers to the initiation or propagation of signaling by an OR polypeptide. OR signaling activity is monitored by measuring a detectable step in a signaling cascade; and more specifically the measurement of cAMP. Alternatively, the measurable activity can be measured indirectly, as in, for example, a reporter gene assay. For most of these assays, kits are commercially available.

As used herein, an “agonist” is a ligand which binds to a receptor and mimic the intracellular response induced by a natural ligand or another identify agonist, for example a new compound present in libraries.

As used herein, the term “binding” refers to the physical association of a molecule (e.g., a ligand such as an organic compound from the formulation described below (Markush)) with a receptor (e.g., the OR7C1 defined herein). As the term is used herein, binding is “specific” if it occurs with an EC₅₀ or a Kd of 1 mM or less, generally in the range of 1 mM to 10 nM. For example, binding is specific if the EC₅₀ or Kd is at most 1 mM, for example at most 500 μM, for example at most 100 μM, for example at most 10 μM, for example at most 9.5 μM, for example at most 9 μM, for example at most 8.5 μM, for example at most 8 μM, for example at most 7.5 μM, for example at most 7 μM, for example at most 6.5 μM, for example at most 6 μM, for example at most 5.5 μM, for example at most 5 μM, for example at most 4.5 μM, for example at most 4 μM, for example at most 3.5 μM, for example at most 3 μM, for example at most 2.5 μM, for example at most 2 μM, for example at most 1.5 μM, for example at most 1 μM, for example at most 750 nM, for example at most 500 nM, for example at most 250 nM or for example at most 100 nM or less.

As used herein, the term “EC₅₀” refers to that concentration of a compound at which a given activity, including binding of a compounds of inventions or other ligands and a functional activity of an OR, is 50% of the maximum for that OR activity measurable using the same assay in the absence of compound. Stated differently, the “EC₅₀” is the concentration of compound that gives 50% activation, when 100% activation is set at the amount of activity that does not increase with the addition of more agonist.

The terms “increase” and “decrease” refer to a change in amount of melanin content present in the melanocytes. An “increase” or “decrease” in melanin production in melanocytes is preferably measured in response to contacting OR7C1 as defined herein present on melanocytes or skin explants with compounds for use in the invention, wherein the change in melanin content is relative to the level content of melanin present in the melanocytes in absence of the compound of invention or in presence of 50 μM of 3,4-dihydroxyphenylalanine (DOPA).

In the present specification, the term “non-therapeutic” refers to the concept which excludes medical practice, that is, treatments to human body by a medical therapy.

The terms described herein and others used in the specification are well understood to those in the art.

Preferred statements (features) and embodiments of the methods, compositions, and uses of this invention are set herein below. Each statement and embodiment of the invention so defined may be combined with any other statement and/or embodiment, unless clearly indicated to the contrary. In particular, any feature indicated as being preferred or advantageous may be combined with any other features or statements indicated as being preferred or advantageous. Hereto, the present invention is in particular captured by any one or any combination of one or more of the below numbered statements and embodiments, with any other aspect and/or embodiment.

1. A tanning composition comprising
(a) a cosmetically acceptable carrier, diluent and/or excipient and
(b) at least one tanning compound selected from the group comprising a compound of formula (I), (II), (III) and (IV), or a racemate, a stereoisomer, a tautomer, a salt, and/or a solvate thereof, including mixtures thereof,

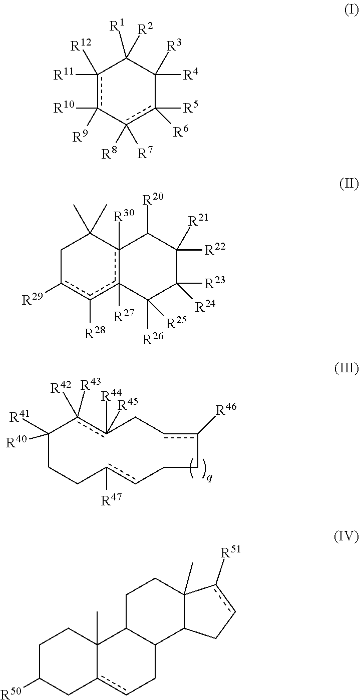

wherein the dash bond [symbol] represents an optional double bond; and wherein
R¹, R² are each independently selected from hydrogen or C₁₋₆alkyl;
R³, R⁴ are each independently selected from hydrogen or C₁₋₆alkyl;
or R¹ with R³ together with the carbon atoms to which they are attached form a saturated C3 to C5-membered ring, wherein said ring is optionally substituted with one or more C₁₋₆alkyl substituents;
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —X—[CR¹³R¹⁴]ₙ—O—[CR¹⁷R¹⁸]ₘ—OH, —([CR¹⁷R¹⁸]ₘ—O)_{y}—C₁₋₆alkyl, —[CR¹³R¹⁴]ₙ—CO—R¹⁹;
m is an integer selected from 1, 2, 3, 4 or 5, n is an integer selected from 0, 1, 2, 3 or 4; y is an integer selected from 1, 2 or 3;
X is a single bond or O,
R¹³, R¹⁴, R¹⁶, R¹⁷, R¹⁸, R¹⁹ are each independently selected from hydrogen or C₁₋₆alkyl;
R¹⁵ is OH, or R¹⁵ together with R¹⁶ form an oxo group;
or R³ and R⁵ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2;
or R⁵ and R⁶ form together ═[CR¹⁴]—[CR¹⁷R¹⁸]ₘ—OH; or R⁵ and R⁶ together with the carbon atom to which they are attached form a 5- or 6-membered heterocyclyl moiety, wherein said heterocyclyl is optionally substituted with one or more C₁₋₆alkyl substituents; or R⁵ and R⁸ together with the carbon atoms to which they are attached form a saturated or unsaturated C5 or C6-membered ring, wherein said ring is optionally substituted with one or more substituents each independently selected from oxo, C₁₋₆alkyl, —O—C₁₋₆alkyl, —CO—C₁₋₆alkyl, —[CR¹⁷R¹⁸]ₘ—O—CO—C₁₋₆alkyl, —([CR¹⁷R¹⁸]ₘ—O)_{y}—C₁₋₆alkyl, —[CR¹⁷R¹⁸]ₘ—COH, or —[CR¹⁷R¹⁸]ₘ—OH; or two substituents on said ring form together with the carbon atoms to which they are attached a 3- to 6-membered heterocyclyl moiety, wherein said heterocyclyl moiety is optionally substituted with one or more substituents each independently selected from C₁₋₆ alkyl or oxo, or two substituents on said heterocyclyl form together with the atoms to which they are attached a 3- to 6-membered heterocyclyl moiety;
or R⁶ and R⁹ together with the carbon atoms to which they are attached form a C3-membered ring;
R⁶ if present is hydrogen or C₁₋₆alkyl;
R⁷ if present is hydrogen or C₁₋₆alkyl;
R⁸ is hydrogen or C₁₋₆alkyl;
or R¹ with R⁸ together with the carbon atoms to which they are attached form a saturated C5-membered ring, wherein said ring is optionally substituted with one or more C₁₋₆alkyl substituents;
R⁹, R¹⁰ are each independently selected from hydrogen or C₁₋₆alkyl;
or R¹ with R⁹ together with the carbon atoms to which they are attached form a saturated C5-membered ring, wherein said ring is optionally substituted with one or more C₁₋₆alkyl substituents;
or R³ with R⁹ together with the carbon atoms to which they are attached form a saturated C5-membered ring, wherein said ring is optionally substituted with one or more C₁₋₆alkyl substituents;
R¹¹, R¹² are each independently selected from hydrogen or C₁₋₆alkyl;
or R⁹ and R¹¹ together with the carbon atoms to which they are attached form a saturated or unsaturated C5 or C6-membered ring, wherein said ring is optionally substituted with one or more C₁₋₆alkyl substituents;
R²⁰ is hydrogen or C₁₋₆alkyl;
R²¹ is hydrogen or C₁₋₆alkyl;
R²² is selected from the group comprising hydrogen, C₁₋₆alkyl, and —CO—R¹⁹;
R²³ is hydrogen or C₁₋₆alkyl;
R²⁴ is hydrogen or C₁₋₆alkyl;
or R²³ and R²⁴ together with the carbon atom to which they are attached form a 3- to 6-membered heterocyclyl moiety; wherein said heterocyclyl is optionally substituted with one or more substituents each independently selected from C₁₋₆alkyl;
R²⁵ is hydrogen or C₁₋₆alkyl;
or R²⁴ and R²⁵ form together with the carbon atom to which they are attached a 5 or 6-membered heterocyclyl moiety, wherein said heterocyclyl is optionally substituted with one or more substituents each independently selected from C₁₋₆alkyl or oxo, or wherein one of said substituent and R²³ form together with the atoms to which they are attached a 3- to 6-membered heterocyclyl moiety;
R²⁶ is hydrogen or C₁₋₆alkyl;
R²⁷ if present is hydrogen or C₁₋₆alkyl;
or R²⁰ and R²⁷ together with the carbon atoms to which they are attached form a C3-membered ring;
R²⁸ is selected from the group comprising hydrogen, oxo, C₁₋₆alkyl, —O—C₁₋₆alkyl, —[CR¹⁷R¹⁸]ₘ—O—CO—C₁₋₆alkyl, —[CR¹⁷R¹⁸]ₘ—O—C₁₋₆alkyl, —[CR¹⁷R¹⁸]ₘ—COH, and —[CR¹⁷R¹⁸]ₘ—OH;
R²⁹ is hydrogen or C₁₋₆alkyl;
R³⁰ if present is hydrogen or C₁₋₆alkyl;
or R³⁰ with R²³ together with the carbon atoms to which they are attached form a saturated C3 to C5-membered ring, wherein said ring is optionally substituted with one or more C₁₋₆alkyl substituents;
or R²⁹ and R²⁸ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; q is an integer selected from 0, 1, 2, 3, preferably q is 1;
R⁴⁰ is selected from the group comprising —[CR¹⁷R¹⁸]ₘ—OH, —CO—C₁₋₆alkyl, —O—([CR¹⁷R¹⁸]ₘ—O—)ₜ—C₁₋₆alkyl, t is an integer selected from 0, 1, 2, or 3;
R⁴¹, R⁴², R⁴³, R⁴⁴, R⁴⁵, R⁴⁶, R⁴⁷ are each independently selected from hydrogen or C₁₋₆alkyl; or R⁴⁰ and R⁴² together with the carbon atom to which they are attached form a 3- to 6-membered heterocyclyl moiety;
R⁵⁰ is selected from OH or oxo; and
R⁵¹ is hydrogen or C₁₋₁₀ alkyl.

2. The tanning composition according to statement 1, wherein the compound is of formula (Ia), (Ib), (Ic), (Id), (IIa), (IIIa), or (IVa),

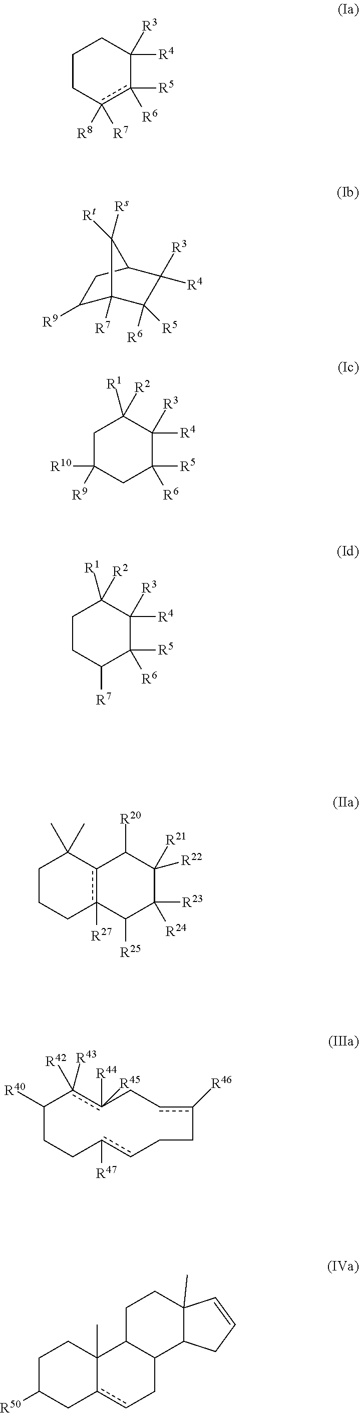

wherein
R³, R⁴, R⁵, R⁶, R⁷, R⁸, R⁹, R²⁰, R²¹, R²², R²³, R²⁴, R²⁵, R²⁷, R⁴⁰, R⁴², R⁴³, R⁴⁴, R⁴⁵, R⁴⁶, R⁴⁷, R⁵⁰ are as defined in statement 1 and R^{t} and R^{s} are each independently selected from hydrogen or C₁₋₆alkyl.

3. The tanning composition according to any one of statements 1-2, wherein the compound is of formula (Ie),

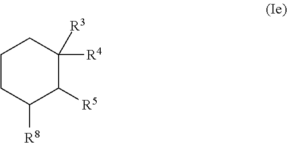

wherein
R³, R⁴ are each independently selected from hydrogen or C₁₋₆alkyl; preferably R³, R⁴ are each independently selected from hydrogen or C₁₋₄alkyl; preferably R³, R⁴ are each independently selected from hydrogen or C₁₋₂alkyl; preferably R³, R⁴ are each independently selected from hydrogen or methyl;
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ᵣ—[CR¹⁷R¹⁸]ₘ—OH, —CO—R¹⁹; preferably R⁵ is selected from the group comprising —X—[CHR¹⁴]ₙ—C(═O)—[CHR¹⁸]ₘ—R¹⁹, —X—[CHR¹⁴]ₙ—CH(OH)—[CHR¹⁸]ₘ—R¹⁹, —X—[CHR¹⁴]ₙ—[CHR¹⁸]ₘ—OH, —X—[CHR¹⁴]ₙ—O—[CHR¹⁸]ₘ—OH, —[CHR¹⁴]ₙ—CO—R¹⁹; preferably R⁵ is selected from the group comprising —O—[CHR¹⁴]ₙ—C(═O)—[CHR¹⁸]ₘ—R¹⁹, —[CHR¹⁴]ₙ—C(═O)—[CHR¹⁸]ₘ—R¹⁹, —O—[CHR¹⁴]ⁿ—CH(OH)—[CHR¹⁸]ₘ—R¹⁹, —[CHR¹⁴]ₙ—CH(OH)—[CHR¹⁸]ₘ—R¹⁹, —O—[CHR¹⁴]ₙ—[CHR¹⁸]ₘ—OH, —[CHR¹⁴]ₙ—[CHR¹⁸]ₘ—OH, —CO—R¹⁹;
m is an integer selected from 1, 2, 3, 4 or 5, n is an integer selected from 0, 1, 2, 3 or 4; preferably m is selected from 1, 2 or 3, preferably m is 1 or 2, preferably n is selected from 0, 1, 2, or 3; preferably n is selected from 0, 1, or 2;
X is a single bond or O,
R¹³, R¹⁴, R¹⁶, R¹⁷, R¹⁸, R¹⁹ are each independently selected from hydrogen or C₁₋₆alkyl; preferably R¹³, R¹⁴, R¹⁶, R¹⁷, R¹⁸, R¹⁹ are each independently selected from hydrogen or C₁₋₄ alkyl; preferably R¹³, R¹⁴, R¹⁶, R¹⁷, R¹⁸, R¹⁹ are each independently selected from hydrogen or C₁₋₂alkyl;
R¹⁵ is OH, or R¹⁵ together with R¹⁶ form an oxo group; and
R⁸ is hydrogen or C₁₋₆alkyl; preferably R⁸ is hydrogen or C₁₋₅alkyl; preferably R⁸ is hydrogen or C₁₋₄alkyl.

4. The tanning composition according to any one of statements 1-2, wherein the compound is of formula (If),

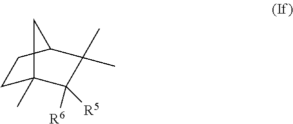

wherein
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —X—[CR¹³R¹⁴]ₙ—O—[CR¹⁷R¹⁸]ₘ—OH, —([CR¹⁷R¹⁸]ₘ—O)_{y}—C₁₋₆alkyl, —[CR¹³R¹⁴]ₙ—CO—R¹⁹; preferably R⁵ is selected from the group comprising —X—[CHR¹⁴]ₙ—C(═O)—[CHR¹⁸]ₘ—R¹⁹, —X—[CHR¹⁴]ₙ—CH(OH)—[CHR¹⁸]ₘ—R¹⁹, —X—[CHR¹⁴]ₙ—[CHR¹⁸]ₘ—OH, —X—[CHR¹⁴]ₙ—O—[CHR¹⁸]ₘ—OH, —([CHR¹⁸]ₘ—O)_{y}—C₁₋₆alkyl, —[CHR¹⁴]ₙ—CO—R¹⁹; preferably R⁵ is selected from the group comprising —O—[CHR¹⁴]ₙ—C(═O)—[CHR¹⁸]ₘ—R¹⁹, —[CHR¹⁴]ₙ—C(═O)—[CHR¹⁸]ₘ—R¹⁹, —O—[CHR¹⁴]ₙ—CH(OH)—[CHR¹⁸]ₘ—R¹⁹, —[CHR¹⁴]ₙ—CH(OH)—[CHR¹⁸]ₘ—R¹⁹, —O—[CHR¹⁴]ₙ—[CHR¹⁸]ₘ—OH, —[CHR¹⁴]ₙ—[CHR¹⁸]ₘ—OH, —CO—R¹⁹;
m is an integer selected from 1, 2, 3, 4 or 5, n is an integer selected from 0, 1, 2, 3 or 4; y is an integer selected from 1, 2 or 3; preferably m is selected from 1, 2 or 3, preferably m is 1 or 2, preferably n is selected from 0, 1, 2, or 3; preferably n is selected from 0, 1, or 2; preferably y is 1, or 2;
X is a single bond or O,
R¹³, R¹⁴, R¹⁶, R¹⁷, R¹⁸, R¹⁹ are each independently selected from hydrogen or C₁₋₆alkyl; preferably R¹³, R¹⁴, R¹⁶, R¹⁷, R¹⁸, R¹⁹ are each independently selected from hydrogen or C₁₋₄ alkyl; preferably R¹³, R¹⁴, R¹⁶, R¹⁷, R¹⁸, R¹⁹ are each independently selected from hydrogen or C₁₋₂alkyl;
R¹⁵ is OH, or R¹⁵ together with R¹⁶ form an oxo group; and
R⁶ is hydrogen or C₁₋₆alkyl; preferably R⁶ is hydrogen or C₁₋₄alkyl; preferably R⁶ is hydrogen or C₁₋₂alkyl; preferably R⁶ is hydrogen or methyl;
or R⁵ and R⁶ form together ═[CR¹⁴]—[CR¹⁷R¹⁸]ₘ—OH; preferably R⁵ and R⁶ form together ═[CH]—[CHR¹⁸]ₘ—OH; and m is 1 or 2; preferably R⁵ and R⁶ form together ═[CH]—[CH₂]ₘ—OH; and m is 1 or 2.

5. The tanning composition according to any one of statements 1-2, wherein the compound is of formula (Ig),

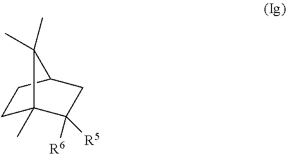

wherein
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —X—[CR¹³R¹⁴]ₙ—O—[CR¹⁷R¹⁸]ₘ—OH, —([CR¹⁷R¹⁸]ₘ—O)_{y}—C₁₋₆alkyl, —[CR¹³R¹⁴]ₙ—CO—R¹⁹; preferably R⁵ is selected from the group comprising —X—[CHR¹⁴]ₙ—C(═O)—[CHR¹⁸]ₘ—R¹⁹, —X—[CHR¹⁴]ₙ—CH(OH)—[CHR¹⁸]ₘ—R¹⁹, —X—[CHR¹⁴]ₙ—[CHR¹⁸]ₘ—OH, —X—[CHR¹⁴]ₙ—O—[CHR¹⁸]ₘ—OH, —([CHR¹⁸]ₘ—O)_{y}—C₁₋₆alkyl, —[CHR¹⁴]ₙ—CO—R¹⁹; preferably R⁵ is selected from the group comprising —O—[CHR¹⁴]ₙ—C(═O)—[CHR¹⁸]ₘ—R¹⁹, —[CHR¹⁴]ₙ—C(═O)—[CHR¹⁸]ₘ—R¹⁹, —O—[CHR¹⁴]ₙ—CH(OH)—[CHR¹⁸]ₘ—R¹⁹, —[CHR¹⁴]ₙ—CH(OH)—[CHR¹⁸]ₘ—R¹⁹, —O—[CHR¹⁴]ₙ—[CHR¹⁸]ₘ—OH, —[CHR¹⁴]ₙ—[CHR¹⁸]ₘ—OH, —CO—R¹⁹;
m is an integer selected from 1, 2, 3, 4 or 5, n is an integer selected from 0, 1, 2, 3 or 4; y is an integer selected from 1, 2 or 3; preferably m is selected from 1, 2 or 3, preferably m is 1 or 2, preferably n is selected from 0, 1, 2, or 3; preferably n is selected from 0, 1, or 2; preferably y is 1, or 2;
X is a single bond or O,
R¹³, R¹⁴, R¹⁶, R¹⁷, R¹⁸, R¹⁹ are each independently selected from hydrogen or C₁₋₆alkyl; preferably R¹³, R¹⁴, R¹⁶, R¹⁷, R¹⁸, R¹⁹ are each independently selected from hydrogen or C₁₋₄alkyl; preferably R¹³, R¹⁴, R¹⁶, R¹⁷, R¹⁸, R¹⁹ are each independently selected from hydrogen or C₁₋₂alkyl;
R¹⁵ is OH, or R¹⁵ together with R¹⁶ form an oxo group;
R⁶ is hydrogen or C₁₋₆alkyl; preferably R⁶ is hydrogen or C₁₋₄alkyl; preferably R⁶ is hydrogen or C₁₋₂alkyl; preferably R⁶ is hydrogen or methyl;
or R⁵ and R⁶ form together ═[CR¹⁴]—[CR¹⁷R¹⁸]ₘ—OH; or R⁵ and R⁶ together with the carbon atom to which they are attached form a 5- or 6-membered heterocyclyl moiety, wherein said heterocyclyl is optionally substituted with one or more C₁₋₆alkyl substituents; preferably R⁵ and R⁶ form together ═[CH]—[CHR¹⁸]ₘ—OH; and m is 1 or 2; or R⁵ and R⁶ together with the carbon atom to which they are attached form a 6-membered heterocyclyl moiety, wherein said heterocyclyl is optionally substituted with one or more C₁₋₆alkyl substituents; preferably R⁵ and R⁶ form together ═[CH]—[CH₂]ₘ—OH; and m is 1 or 2; or R⁵ and R⁶ together with the carbon atom to which they are attached form a 6-membered dioxanyl moiety, for example 1,3-dioxanyl, wherein said dioxanyl is optionally substituted with one or more C₁₋₆alkyl substituents, for example one or more C₁₋₄alkyl substituents, one or more C₁₋₃alkyl substituents.

6. The tanning composition according to any one of statements 1-2, wherein the compound is of formula (IIb),

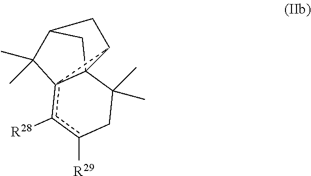

wherein the dash bond [symbol] represents an optional double bond; and wherein [symbol] represent an optional single bond;
R²⁸ is selected from the group comprising hydrogen, oxo, C₁₋₆alkyl, —O—C₁₋₆alkyl, —[CR¹⁷R¹⁸]ₘ—O—CO—C₁₋₆alkyl, —[CR¹⁷R¹⁸]ₘ—O—C₁₋₆alkyl, —[CR¹⁷R¹⁸]ₘ—COH, and —[CR¹⁷R¹⁸]ₘ—OH; m is 1, 2, 3 or 4, and R¹⁷ and R¹⁸ are each independently selected from hydrogen or C₁₋₆alkyl; preferably R²⁸ is selected from the group comprising hydrogen, oxo, C₁₋₄alkyl, —O—C₁₋₄alkyl, —[CHR¹⁸]ₘ—O—CO—C₁₋₄alkyl, —[CHR¹⁸]ₘ—O—C₁₋₄alkyl, —[CHR¹⁸]ₘ—COH, and —[CHR¹⁸]ₘ—OH, m is 1, 2, or 3, and R¹⁸ is hydrogen or C₁₋₄alkyl; preferably R²⁸ is selected from the group comprising hydrogen, oxo, C₁₋₃ alkyl, —O—C₁₋₃alkyl, —[CHR¹⁸]ₘ—O—CO—C₁₋₃alkyl, —[CHR¹⁸]ₘ—O—C₁₋₃alkyl, —[CHR¹⁸]ₘ—COH, and —[CHR¹⁸]ₘ—OH, m is 1, 2, or 3, and R¹⁸ is hydrogen or C₁₋₃alkyl;
R²⁹ is hydrogen or C₁₋₆alkyl; preferably R²⁹ is hydrogen or C₁₋₄alkyl; preferably R²⁹ is hydrogen or C₁₋₂alkyl; preferably R²⁹ is hydrogen or methyl;
or R²⁹ and R²⁸ together are of formula —O—[CR¹⁷R¹⁸]ₚ—O—, p is an integer selected from 1 or 2; and form together with the carbon atoms to which they are attached a 5- or 6-membered heterocyclyl moiety, wherein said heterocyclyl is optionally substituted with one or more C₁₋₆ alkyl substituents, and R¹⁷ and R¹⁸ are each independently selected from hydrogen or C₁₋₆ alkyl; preferably R²⁹ and R²⁸ together are of formula —O—[CR¹⁷R¹⁸]—O—; and form together with the carbon atoms to which they are attached a dioxolanyl, wherein said dioxolanyl is optionally substituted with one or more C₁₋₄alkyl substituents, and R¹⁷ and R¹⁸ are each independently selected from hydrogen or C₁₋₄alkyl; preferably R²⁹ and R²⁸ together are of formula —O—[CR¹⁷R¹⁸]—O—; and form together with the carbon atoms to which they are attached a dioxolanyl, wherein said dioxolanyl is optionally substituted with one or more C₁₋₂alkyl substituents, for example C₁₋₂alkyl substituents; R¹⁷ and R¹⁸ are each independently selected from hydrogen or C₁₋₂alkyl.

7. The tanning composition according to any one of statements 1-2, wherein the compound is of

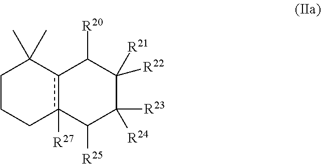

wherein the dash bond [symbol] represents an optional double bond; and wherein
R²⁰ is hydrogen or C₁₋₆alkyl; preferably R²⁰ is hydrogen or C₁₋₄alkyl; preferably R²⁰ is hydrogen or C₁₋₂alkyl; preferably R²⁰ is hydrogen or methyl;
R²¹ is hydrogen or C₁₋₆alkyl; preferably R²¹ is hydrogen or C₁₋₄alkyl; preferably R²¹ is hydrogen or C₁₋₂alkyl; preferably R²¹ is hydrogen or methyl;
R²² is selected from the group comprising hydrogen, C₁₋₆alkyl, and —CO—R¹⁹; with R¹⁹ being hydrogen or C₁₋₆alkyl; preferably R²² is selected from the group comprising hydrogen, C₁₋₄alkyl, and —CO—R¹⁹; with R¹⁹ being hydrogen or C₁₋₄alkyl; preferably R²² is selected from the group comprising hydrogen, C₁₋₂alkyl, and —CO—R¹⁹; with R¹⁹ being hydrogen or C₁₋₂alkyl; preferably R²² is selected from the group comprising hydrogen, methyl, and —CO-methyl;
R²³ is hydrogen or C₁₋₆alkyl; preferably R²³ is hydrogen or C₁₋₄alkyl; preferably R²³ is hydrogen, methyl or ethyl;
R²⁴ is hydrogen or C₁₋₆alkyl; preferably R²⁴ is hydrogen or C₁₋₄alkyl; preferably R²⁴ is hydrogen or C₁₋₂alkyl; preferably R²⁴ is hydrogen or methyl;
or R²³ and R²⁴ together with the carbon atom to which they are attached form a 3- to 6-membered heterocyclyl moiety; wherein said heterocyclyl is optionally substituted with one or more substituents each independently selected from C₁₋₆alkyl; preferably R²³ and R²⁴ together with the carbon atom to which they are attached form a 3- to 5-membered heterocyclyl moiety; wherein said heterocyclyl is optionally substituted with one or more C₁₋₄alkyl substituents; preferably R²³ and R²⁴ together with the carbon atom to which they are attached form a 3- to 4-membered heterocyclyl moiety; wherein said heterocyclyl is optionally substituted with one or more C₁₋₂alkyl substituents; preferably R²³ and R²⁴ together with the carbon atom to which they are attached form a 3-heterocyclyl moiety, preferably oxiranyl; wherein said heterocyclyl is optionally substituted with one or more C₁₋₂alkyl substituents, preferably methyl;
R²⁵ is hydrogen or C₁₋₆alkyl; preferably R²⁵ is hydrogen or C₁₋₄alkyl; preferably R²⁵ is hydrogen or C₁₋₂alkyl; preferably R²⁵ is hydrogen or methyl;
or R²⁴ and R²⁵ form together with the carbon atom to which they are attached a 5 or 6-membered heterocyclyl moiety, wherein said heterocyclyl is optionally substituted with one or more substituents each independently selected from C₁₋₆alkyl or oxo, or wherein one of said substituent and R²³ form together with the atoms to which they are attached a 3- to 6-membered heterocyclyl moiety; preferably R²⁴ and R²⁵ form together with the carbon atom to which they are attached a 5 or 6-membered heterocyclyl moiety, wherein said heterocyclyl is optionally substituted with one or more substituents each independently selected from C₁₋₄alkyl or oxo, or wherein one of said substituent and R²³ form together with the atoms to which they are attached a 4- to 6-membered heterocyclyl moiety; preferably R²⁴ and R²⁵ form together with the carbon atom to which they are attached a 5 or 6-membered heterocyclyl moiety for example a tetrahydropyranyl or a tetrahydrofuranyl, wherein said heterocyclyl is optionally substituted with one or more substituents each independently selected from C₁₋₂alkyl or oxo, or wherein one of said substituent and R²³ form together with the atoms to which they are attached a 5- to 6-membered heterocyclyl moiety such as a dioxolanyl;
R²⁷ if present is hydrogen or C₁₋₆alkyl; preferably R²⁷ is hydrogen or C₁₋₄alkyl; preferably R²⁷ is hydrogen or C₁₋₂alkyl; preferably R²⁷ is hydrogen or methyl.

8. The tanning composition according to any one of statements 1, 2, 7, wherein the compound is of formula (IIa),
wherein the dash bond [symbol] represents an optional double bond; and wherein
R²⁰ is hydrogen or C₁₋₆alkyl; preferably R²⁰ is hydrogen or C₁₋₄alkyl; preferably R²⁰ is hydrogen or C₁₋₂alkyl; preferably R²⁰ is hydrogen or methyl;
R²¹ is hydrogen or C₁₋₆alkyl; preferably R²¹ is hydrogen or C₁₋₄alkyl; preferably R²¹ is hydrogen or C₁₋₂alkyl; preferably R²¹ is hydrogen or methyl;
R²² is selected from the group comprising hydrogen, C₁₋₆alkyl, and —CO—R¹⁹; with R¹⁹ being hydrogen or C₁₋₆alkyl; preferably R²² is selected from the group comprising hydrogen, C₁₋₄alkyl, and —CO—R¹⁹; with R¹⁹ being hydrogen or C₁₋₄alkyl; preferably R²² is selected from the group comprising hydrogen, C₁₋₂alkyl, and —CO—R¹⁹; with R¹⁹ being hydrogen or C₁₋₂alkyl; preferably R²² is selected from the group comprising hydrogen, methyl, and —CO-methyl;
R²³ is hydrogen or C₁₋₆alkyl; preferably R²³ is hydrogen or C₁₋₄alkyl; preferably R²³ is hydrogen, methyl or ethyl;
R²⁴ is hydrogen or C₁₋₆alkyl; preferably R²⁴ is hydrogen or C₁₋₄alkyl; preferably R²⁴ is hydrogen or C₁₋₂alkyl; preferably R²⁴ is hydrogen or methyl;
or R²³ and R²⁴ together with the carbon atom to which they are attached form a 3- to 6-membered heterocyclyl moiety containing one or two O atoms, the rest being carbon atoms; wherein said heterocyclyl is optionally substituted with one or more substituents each independently selected from C₁₋₆alkyl; preferably R²³ and R²⁴ together with the carbon atom to which they are attached form a 3- to 5-membered heterocyclyl moiety containing one or two O atoms, the rest being carbon atoms; wherein said heterocyclyl is optionally substituted with one or more C₁₋₄alkyl substituents; preferably R²³ and R²⁴ together with the carbon atom to which they are attached form a 3- to 4-membered heterocyclyl moiety containing one O atom, the rest being carbon atoms; wherein said heterocyclyl is optionally substituted with one or more C₁₋₂alkyl substituents; preferably R²³ and R²⁴ together with the carbon atom to which they are attached form an oxiranyl; wherein said oxiranyl is optionally substituted with one or more C₁₋₂alkyl substituents, preferably methyl;
R²⁵ is hydrogen or C₁₋₆alkyl; preferably R²⁵ is hydrogen or C₁₋₄alkyl; preferably R²⁵ is hydrogen or C₁₋₂alkyl; preferably R²⁵ is hydrogen or methyl;
or R²⁴ and R²⁵ form together with the carbon atom to which they are attached a 5 or 6-membered heterocyclyl moiety containing one or two O atoms, the rest being carbon atoms; wherein said heterocyclyl is optionally substituted with one or more substituents each independently selected from C₁₋₆alkyl or oxo, or wherein one of said substituent and R²³ form together with the atoms to which they are attached a 3- to 6-membered heterocyclyl moiety containing one or two O atoms, the rest being carbon atoms; preferably R²⁴ and R²⁵ form together with the carbon atom to which they are attached a 5 or 6-membered heterocyclyl moiety containing one or two O atoms, the rest being carbon atoms, wherein said heterocyclyl is optionally substituted with one or more substituents each independently selected from C₁₋₄ alkyl or oxo, or wherein one of said substituent and R²³ form together with the atoms to which they are attached a 4- to 6-membered heterocyclyl moiety containing one or two O atoms, the rest being carbon atoms; preferably R²⁴ and R²⁵ together with the carbon atom to which they are attached form a tetrahydropyranyl or a tetrahydrofuranyl, wherein said tetrahydropyranyl or a tetrahydrofuranyl is optionally substituted with one or more substituents each independently selected from C₁₋₂alkyl or oxo, or wherein one of said substituent and R²³ form together with the atoms to which they are attached a 5- to 6-membered heterocyclyl moiety containing one or two O atoms, the rest being carbon atoms; such as a dioxolanyl;
R²⁷ if present is hydrogen or C₁₋₆alkyl; preferably R²⁷ is hydrogen or C₁₋₄alkyl; preferably R²⁷ is hydrogen or C₁₋₂alkyl; preferably R²⁷ is hydrogen or methyl.

9. The tanning composition according to any one of statements 1, 2, 7, 8, wherein the compound is of formula (IIa),
wherein the dash bond [symbol] represents an optional double bond; and wherein
R²⁰ is hydrogen or C₁₋₄alkyl; preferably R²⁰ is hydrogen or C₁₋₂alkyl; preferably R²⁰ is hydrogen or methyl;
R²¹ is hydrogen or C₁₋₄alkyl; preferably R²¹ is hydrogen or C₁₋₂alkyl; preferably R²¹ is hydrogen or methyl;
R²² is selected from the group comprising hydrogen, C₁₋₄alkyl, and —CO—R¹⁹; with R¹⁹ being hydrogen or C₁₋₄alkyl; preferably R²² is selected from the group comprising hydrogen, C₁₋₂alkyl, and —CO—R¹⁹; with R¹⁹ being hydrogen or C₁₋₂alkyl; preferably R²² is selected from the group comprising hydrogen, methyl, and —CO-methyl;
R²³ is hydrogen or C₁₋₄alkyl; preferably R²³ is hydrogen, methyl or ethyl;
R²⁴ is hydrogen or C₁₋₄alkyl; preferably R²⁴ is hydrogen or C₁₋₂alkyl; preferably R²⁴ is hydrogen or methyl;
or R²³ and R²⁴ together with the carbon atom to which they are attached form a 3- to 5-membered heterocyclyl moiety containing one or two O atoms, the rest being carbon atoms; wherein said heterocyclyl is optionally substituted with one or more C₁₋₄alkyl substituents; preferably R²³ and R²⁴ together with the carbon atom to which they are attached form a 3- to 4-membered heterocyclyl moiety containing one O atom, the rest being carbon atoms; wherein said heterocyclyl is optionally substituted with one or more C₁₋₂alkyl substituents; preferably R²³ and R²⁴ together with the carbon atom to which they are attached form an oxiranyl; wherein said oxiranyl is optionally substituted with one or more C₁₋₂alkyl substituents, preferably methyl;
R²⁵ is hydrogen or C₁₋₄alkyl; preferably R²⁵ is hydrogen or C₁₋₂alkyl; preferably R²⁵ is hydrogen or methyl;
or R²⁴ and R²⁵ form together with the carbon atom to which they are attached a 5 or 6-membered heterocyclyl moiety containing one or two O atoms, the rest being carbon atoms, wherein said heterocyclyl is optionally substituted with one or more substituents each independently selected from C₁₋₄alkyl or oxo, or wherein one of said substituent and R²³ form together with the atoms to which they are attached a 4- to 6-membered heterocyclyl moiety containing one or two O atoms, the rest being carbon atoms; preferably R²⁴ and R²⁵ together with the carbon atom to which they are attached form a tetrahydropyranyl or a tetrahydrofuranyl, wherein said tetrahydropyranyl or a tetrahydrofuranyl is optionally substituted with one or more substituents each independently selected from C₁₋₂alkyl or oxo, or wherein one of said substituent and R²³ form together with the atoms to which they are attached a 5- to 6-membered heterocyclyl moiety containing one or two 0 atoms, the rest being carbon atoms; such as a dioxolanyl;
R²⁷ is hydrogen or C₁₋₄alkyl; preferably R²⁷ is hydrogen or C₁₋₂alkyl; preferably R²⁷ is hydrogen or methyl.

10. The tanning composition according to any one of statements 1-2, wherein the compound is of formula (Ic),

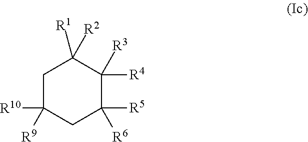

wherein
R¹, R² are each independently selected from hydrogen or C₁₋₆alkyl; preferably R¹, R² are each independently selected from hydrogen or C₁₋₄alkyl; preferably R¹, R² are each independently selected from hydrogen or C₁₋₂alkyl; preferably R¹, R² are each independently selected from hydrogen or methyl;
R³, R⁴ are each independently selected from hydrogen or C₁₋₆alkyl; preferably R³, R⁴ are each independently selected from hydrogen or C₁₋₄alkyl; preferably R³, R⁴ are each independently selected from hydrogen or C₁₋₂alkyl; preferably R³, R⁴ are each independently selected from hydrogen or methyl;
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —X—[CR¹³R¹⁴]ₙ—O—[CR¹⁷R¹⁸]ₘ—OH, —([CR¹⁷R¹⁸]ₘ—O)_{y}—C₁₋₆alkyl, —[CR¹³R¹⁴]ₙ—CO—R¹⁹; preferably R⁵ is selected from the group comprising —X—[CHR¹⁴]ₙ—C(═O)—[CHR¹⁸]ₘ—R¹⁹, —X—[CHR¹⁴]ₙ—CH(OH)—[CHR¹⁸]ₘ—R¹⁹, —X—[CHR¹⁴]ₙ—[CHR¹⁸]ₘ—OH, —X—[CHR¹⁴]ₙ—O—[CHR¹⁸]ₘ—OH, —([CHR¹⁸]ₘ—O)_{y}—C₁₋₆alkyl, —[CHR¹⁴]ₙ—CO—R¹⁹; preferably R⁵ is selected from the group comprising —O—[CHR¹⁴]ₙ—C(═O)—[CHR¹⁸]ₘ—R¹⁹, —[CHR¹⁴]ₙ—C(═O)—[CHR¹⁸]ₘ—R¹⁹, —O—[CHR¹⁴]ₙ—CH(OH)—[CHR¹⁸]ₘ—R¹⁹, —[CHR¹⁴]ₙ—CH(OH)—[CHR¹⁸]ₘ—R¹⁹, —O—[CHR¹⁴]ₙ—[CHR¹⁸]ₘ—OH, —[CHR¹⁴]ₙ—[CHR¹⁸]ₘ—OH, —CO—R¹⁹;
m is an integer selected from 1, 2, 3, 4 or 5, n is an integer selected from 0, 1, 2, 3 or 4; y is an integer selected from 1, 2 or 3; preferably m is selected from 1, 2 or 3, preferably m is 1 or 2, preferably n is selected from 0, 1, 2, or 3; preferably n is selected from 0, 1, or 2; preferably y is 1, or 2;
X is a single bond or O,
R¹³, R¹⁴, R¹⁶, R¹⁷, R¹⁸, R¹⁹ are each independently selected from hydrogen or C₁₋₆alkyl; preferably R¹³, R¹⁴, R¹⁶, R¹⁷, R¹⁸, R¹⁹ are each independently selected from hydrogen or C₁₋₄alkyl; preferably R¹³, R¹⁴, R¹⁶, R¹⁷, R¹⁸, R¹⁹ are each independently selected from hydrogen or C₁₋₂alkyl;
R¹⁵ is OH, or R¹⁵ together with R¹⁶ form an oxo group;
or R³ and R⁵ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2, R¹⁷, R¹⁸, are each independently selected from hydrogen or C₁₋₆alkyl; preferably R³ and R⁵ together are of formula —O—[CR¹⁷R¹⁸]—O—, and form together with the carbon atoms to which they are attached a 5-membered heterocyclyl moiety, wherein said heterocyclyl is optionally substituted with one or more C₁₋₆alkyl substituents; preferably R³ and R⁵ together are of formula —O—[CR¹⁷R¹⁸]—O—, and form together with the carbon atoms to which they are attached a dioxolanyl moiety, R¹⁷, R¹⁸, are each independently selected from hydrogen or C₁₋₄alkyl, wherein said dioxolanyl moiety is optionally substituted with one or two C₁₋₄alkyl substituents, preferably one or two C₁₋₂alkyl substituents, preferably one or two methyl;
R⁶ is hydrogen or C₁₋₆alkyl; preferably R⁶ is hydrogen or C₁₋₄alkyl; preferably R⁶ is hydrogen or C₁₋₂alkyl; preferably R⁶ is hydrogen or methyl;
R⁹, R¹⁰ are each independently selected from hydrogen or C₁₋₆alkyl; preferably R⁹, R¹⁰ are each independently selected from hydrogen or C₁₋₄alkyl; R⁹, R¹⁰ are each independently selected from hydrogen or C₁₋₂alkyl;
or R¹ with R⁹ together with the carbon atoms to which they are attached form a saturated C5-membered ring, wherein said ring is optionally substituted with one or more C₁₋₆alkyl substituents; preferably wherein said ring is optionally substituted with one or more C₁₋₄alkyl substituents; preferably wherein said ring is optionally substituted with one or more C₁₋₂alkyl substituents.

11. The tanning composition according to any one of statements 1, 2, 10, wherein the compound is of formula (Ic), wherein:
R² is hydrogen or C₁₋₆alkyl; preferably R² is hydrogen or C₁₋₄alkyl; preferably R² is hydrogen or C₁₋₂alkyl; preferably R² is hydrogen or methyl, preferably hydrogen;
R⁴ is hydrogen or C₁₋₆alkyl; preferably R⁴ is hydrogen or C₁₋₄alkyl; preferably R⁴ is hydrogen or C₁₋₂alkyl; preferably R⁴ is hydrogen or methyl;
R³ and R⁵ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2, R¹⁷, R¹⁸, are each independently selected from hydrogen or C₁₋₆alkyl; preferably R³ and R⁵ together are of formula —O—[CR¹⁷R¹⁸]—O—, and form together with the carbon atoms to which they are attached a 5-membered heterocyclyl moiety, wherein said heterocyclyl is optionally substituted with one or more C₁₋₆alkyl substituents; preferably R³ and R⁵ together are of formula —O—[CR¹⁷R¹⁸]—O—, and form together with the carbon atoms to which they are attached a dioxolanyl moiety, R¹⁷, R¹⁸, are each independently selected from hydrogen or C₁₋₄alkyl, wherein said dioxolanyl moiety is optionally substituted with one or two C₁₋₄alkyl substituents, preferably one or two C₁₋₂alkyl substituents, preferably one or two methyl;
R⁶ is hydrogen or C₁₋₆alkyl; preferably R⁶ is hydrogen or C₁₋₄alkyl; preferably R⁶ is hydrogen or C₁₋₂alkyl; preferably R⁶ is hydrogen or methyl;
R¹⁰ is hydrogen or C₁₋₆alkyl; preferably R¹⁰ is hydrogen or C₁₋₄alkyl; R¹⁰ is hydrogen or C₁₋₃ alkyl, preferably hydrogen, methyl, ethyl, n-propyl or isopropyl;
and R¹ with R⁹ together with the carbon atoms to which they are attached form a saturated C5-membered ring, wherein said ring is optionally substituted with one or more C₁₋₆alkyl substituents; preferably wherein said ring is optionally substituted with one or more C₁₋₄alkyl substituents; preferably wherein said ring is optionally substituted with one or more C₁₋₂alkyl substituents, preferably wherein said ring is optionally substituted with one or more methyl substituents.

12. The tanning composition according to any one of statements 1, 2, 10, 11, wherein the compound is of formula (Ic), wherein:
R² is hydrogen or C₁₋₄alkyl; preferably R² is hydrogen or C₁₋₂alkyl; preferably R² is hydrogen or methyl, preferably hydrogen;
R⁴ is hydrogen or C₁₋₄alkyl; preferably R⁴ is hydrogen or C₁₋₂alkyl; preferably R⁴ is hydrogen or methyl;
R³ and R⁵ together are of formula —O—[CR¹⁷R¹⁸]—O—, and form together with the carbon atoms to which they are attached a 5-membered heterocyclyl moiety, wherein said heterocyclyl is optionally substituted with one or more C₁₋₆alkyl substituents; preferably R³ and R⁵ together are of formula —O—[CR¹⁷R¹⁸]—O—, and form together with the carbon atoms to which they are attached a dioxolanyl moiety, R¹⁷, R¹⁸, are each independently selected from hydrogen or C₁₋₄ alkyl, wherein said dioxolanyl moiety is optionally substituted with one or two C₁₋₄alkyl substituents, preferably one or two C₁₋₂alkyl substituents, preferably one or two methyl;
R⁶ is hydrogen or C₁₋₄alkyl; preferably R⁶ is hydrogen or C₁₋₂alkyl; preferably R⁶ is hydrogen or methyl;
R¹⁰ is hydrogen or C₁₋₄alkyl; R¹⁰ is hydrogen or C₁₋₃alkyl, preferably hydrogen, methyl, ethyl, n-propyl or isopropyl;
and R¹ with R⁹ together with the carbon atoms to which they are attached form a saturated C5-membered ring, wherein said ring is optionally substituted with one or more C₁₋₆alkyl substituents; preferably wherein said ring is optionally substituted with one or more C₁₋₄alkyl substituents; preferably wherein said ring is optionally substituted with one or more C₁₋₂alkyl substituents, preferably wherein said ring is optionally substituted with one or more methyl substituents.

13. The tanning composition according to any one of statements 1-2, wherein the compound is of formula (Id),

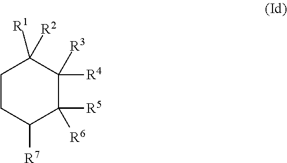

wherein
R¹, R² are each independently selected from hydrogen or C₁₋₆alkyl; preferably R¹, R² are each independently selected from hydrogen or C₁₋₄alkyl; preferably R¹, R² are each independently selected from hydrogen or C₁₋₂alkyl; preferably R¹, R² are each independently selected from hydrogen or methyl;
R³, R⁴ are each independently selected from hydrogen or C₁₋₆alkyl; preferably R³, R⁴ are each independently selected from hydrogen or C₁₋₄alkyl; preferably R³, R⁴ are each independently selected from hydrogen or C₁₋₂alkyl; preferably R³, R⁴ are each independently selected from hydrogen or methyl;
or R¹ with R³ together with the carbon atoms to which they are attached form a saturated C3 to C5-membered ring, wherein said ring is optionally substituted with one or more C₁₋₆alkyl substituents; preferably R¹ with R³ together with the carbon atoms to which they are attached form a C3 to C4-membered ring, wherein said ring is optionally substituted with one or more C₁₋₄alkyl substituents; preferably R¹ with R³ together with the carbon atoms to which they are attached form a C3-membered ring i.e. a cyclopropyl, wherein said cyclopropyl is optionally substituted with one or more C₁₋₂alkyl substituents; preferably wherein said cyclopropyl is optionally substituted with one or more methyl substituents;
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —X—[CR¹³R¹⁴]ₙ—O—[CR¹⁷R¹⁸]ₘ—OH, —([CR¹⁷R¹⁸]ₘ—O)_{y}—C1.6alkyl, —[CR¹³R¹⁴]ₙ—CO—R¹⁹; preferably R⁵ is selected from the group comprising —X—[CHR¹⁴]ₙ—C(═O)—[CHR¹⁸]ₘ—R¹⁹, —X—[CHR¹⁴]ₙ—CH(OH)—[CHR¹⁸]ₘ—R¹⁹, —X—[CHR¹⁴]ₙ—[CHR¹⁸]ₘ—OH, —X—[CHR¹⁴]ₙ—O—[CHR¹⁸]ₘ—OH, —([CHR¹⁸]ₘ—O)_{y}—C₁₋₆alkyl, —[CHR¹⁴]ₙ—CO—R¹⁹; preferably R⁵ is selected from the group comprising —O—[CHR¹⁴]ₙ—C(═O)—[CHR¹⁸]ₘ—R¹⁹, —[CHR¹⁴]ₙ—C(═O)—[CHR¹⁸]ₘ—R¹⁹, —O—[CHR¹⁴]ₙ—CH(OH)—[CHR¹⁸]ₘ—R¹⁹, —[CHR¹⁴]ₙ—CH(OH)—[CHR¹⁸]ₘ—R¹⁹, —O—[CHR¹⁴]ₙ—[CHR¹⁸]ₘ—OH, —[CHR¹⁴]ₙ—[CHR¹⁸]ₘ—OH, —CO—R¹⁹;
m is an integer selected from 1, 2, 3, 4 or 5, n is an integer selected from 0, 1, 2, 3 or 4; y is an integer selected from 1, 2 or 3; preferably m is selected from 1, 2 or 3, preferably m is 1 or 2, preferably n is selected from 0, 1, 2, or 3; preferably n is selected from 0, 1, or 2; preferably y is 1, or 2;
X is a single bond or O,
R¹³, R¹⁴, R¹⁶, R¹⁷, R¹⁸, R¹⁹ are each independently selected from hydrogen or C₁₋₆alkyl; preferably R¹³, R¹⁴, R¹⁶, R¹⁷, R¹⁸, R¹⁹ are each independently selected from hydrogen or C₁₋₄alkyl; preferably R¹³, R¹⁴, R¹⁶, R¹⁷, R¹⁸, R¹⁹ are each independently selected from hydrogen or C₁₋₂alkyl;
R¹⁵ is OH, or R¹⁵ together with R¹⁶ form an oxo group;
R⁶ is hydrogen or C₁₋₆alkyl; preferably R⁶ is hydrogen or C₁₋₄alkyl; preferably R⁶ is hydrogen or C₁₋₂alkyl; preferably R⁶ is hydrogen or methyl;
or R⁵ and R⁶ form together ═[CR¹⁴]—[CR¹⁷R¹⁸]ₘ—OH; or R⁵ and R⁶ together with the carbon atom to which they are attached form a 5- or 6-membered heterocyclyl moiety, wherein said heterocyclyl is optionally substituted with one or more C₁₋₆alkyl substituents; preferably R⁵ and R⁶ form together ═[CH]—[CHR¹⁸]ₘ—OH; and m is 1 or 2; or R⁵ and R⁶ together with the carbon atom to which they are attached form a 6-membered heterocyclyl moiety, wherein said heterocyclyl is optionally substituted with one or more C₁₋₆alkyl substituents; preferably R⁵ and R⁶ form together ═[CH]—[CH₂]ₘ—OH; and m is 1 or 2; or R⁵ and R⁶ together with the carbon atom to which they are attached form a 6-membered dioxanyl moiety, for example 1,3-dioxanyl, wherein said dioxanyl is optionally substituted with one or more C₁₋₆alkyl substituents, for example one or more C₁₋₄alkyl substituents, one or more C₁₋₃alkyl substituents;
R⁷ is hydrogen or C₁₋₆alkyl, preferably R⁷ is hydrogen or C₁₋₄alkyl; preferably R⁷ is hydrogen or C₁₋₂alkyl; preferably R⁷ is hydrogen or methyl.

14. The tanning composition according to any one of statements 1, 2, 13, wherein the compound is of formula (Id), wherein:
R² is hydrogen or C₁₋₆alkyl; preferably R² is hydrogen or C₁₋₄alkyl; preferably R² is hydrogen or C₁₋₂alkyl; preferably R² is hydrogen or methyl, preferably hydrogen;
R⁴ is hydrogen or C₁₋₆alkyl; preferably R⁴ is hydrogen or C₁₋₄alkyl; preferably R⁴ is hydrogen or C₁₋₂alkyl; preferably R⁴ is hydrogen or methyl, preferably hydrogen;
R¹ with R³ together with the carbon atoms to which they are attached form a saturated C3 to C5-membered ring, wherein said ring is optionally substituted with one or more C₁₋₆alkyl substituents; preferably R¹ with R³ together with the carbon atoms to which they are attached form a C3 to C4-membered ring, wherein said ring is optionally substituted with one or more C₁₋₄alkyl substituents; preferably R¹ with R³ together with the carbon atoms to which they are attached form a C3-membered ring i.e. a cyclopropyl, wherein said cyclopropyl is optionally substituted with one or more C₁₋₂alkyl substituents; preferably wherein said cyclopropyl is optionally substituted with one or more methyl substituents;
R⁶ is hydrogen or C₁₋₆alkyl; preferably R⁶ is hydrogen or C₁₋₄alkyl; preferably R⁶ is hydrogen or C₁₋₂alkyl; preferably R⁶ is hydrogen or methyl;
R⁵ and R⁶ together with the carbon atom to which they are attached form a 5- or 6-membered heterocyclyl moiety, wherein said heterocyclyl is optionally substituted with one or more C₁₋₆ alkyl substituents; preferably R⁵ and R⁶ together with the carbon atom to which they are attached form a 6-membered heterocyclyl moiety, wherein said heterocyclyl is optionally substituted with one or more C₁₋₆alkyl substituents; preferably R⁵ and R⁶ together with the carbon atom to which they are attached form a 6-membered dioxanyl moiety, for example 1,3-dioxanyl, wherein said dioxanyl is optionally substituted with one or more C₁₋₆alkyl substituents, for example one or more C₁₋₄alkyl substituents, for example one or more C₁₋₃ alkyl substituents, for example one or more C₁₋₂alkyl substituents, for example one or more methyl substituents; and
R⁷ is hydrogen or C₁₋₆alkyl, preferably R⁷ is hydrogen or C₁₋₄alkyl; preferably R⁷ is hydrogen or C₁₋₂alkyl; preferably R⁷ is hydrogen or methyl, preferably methyl.

15. The tanning composition according to any one of statements 1, 2, 13, 14, wherein the compound is of formula (Id), wherein:
R² is hydrogen or C₁₋₄alkyl; preferably R² is hydrogen or C₁₋₂alkyl; preferably R² is hydrogen or methyl, preferably hydrogen;
R⁴ is hydrogen or C₁₋₄alkyl; preferably R⁴ is hydrogen or C₁₋₂alkyl; preferably R⁴ is hydrogen or methyl, preferably hydrogen;
R¹ with R³ together with the carbon atoms to which they are attached form a C3 to C4-membered ring, wherein said ring is optionally substituted with one or more C₁₋₄alkyl substituents; preferably R¹ with R³ together with the carbon atoms to which they are attached form a C3-membered ring i.e. a cyclopropyl, wherein said cyclopropyl is optionally substituted with one or more C₁₋₂alkyl substituents; preferably wherein said cyclopropyl is optionally substituted with one or more methyl substituents;
R⁶ is hydrogen or C₁₋₄alkyl; preferably R⁶ is hydrogen or C₁₋₂alkyl; preferably R⁶ is hydrogen or methyl;
R⁵ and R⁶ together with the carbon atom to which they are attached form a 6-membered heterocyclyl moiety, wherein said heterocyclyl is optionally substituted with one or more C₁₋₆ alkyl substituents; preferably R⁵ and R⁶ together with the carbon atom to which they are attached form a 6-membered dioxanyl moiety, for example 1,3-dioxanyl, wherein said dioxanyl is optionally substituted with one or more C₁₋₆alkyl substituents, for example one or more C₁₋₄ alkyl substituents, for example one or more C₁₋₃alkyl substituents, for example one or more C₁₋₂alkyl substituents, for example one or more methyl substituents; and
R⁷ is hydrogen or C₁₋₄alkyl; preferably R⁷ is hydrogen or C₁₋₂alkyl; preferably R⁷ is hydrogen or methyl, preferably methyl.

16. The tanning composition according to any one of statements 1-2, wherein the compound is of formula (IIIa),

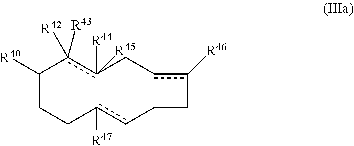

wherein the dash bond [symbol] represents an optional double bond; and wherein
R⁴⁰ is selected from the group comprising —[CR¹⁷R¹⁸]ₘ—OH, —CO—C₁₋₆alkyl, —O—([CR¹⁷R¹⁸]ₘ—O—)ₜ—C₁₋₆alkyl, t is an integer selected from 0, 1, 2, or 3; m is an integer selected from 1, 2, 3 or 4, and R¹⁷ and R¹⁸ are each independently selected from hydrogen or C₁₋₆alkyl; preferably R⁴⁰ is selected from the group comprising —[CHR¹³]ₘ—OH, —CO—C₁₋₆alkyl, —O—([CHR¹³]ₘ—O—)ₜ—C₁₋₆alkyl, t is an integer selected from 0, 1, or 2; m is an integer selected from 1, 2, or 3, and R¹⁸ is hydrogen or C₁₋₆alkyl; preferably R⁴⁰ is selected from the group comprising —[CHR¹³]ₘ—OH, —CO—C₁₋₄alkyl, —O—([CHR¹³]ₘ—O—)ₜ—C₁₋₄alkyl, t is an integer selected from 0, or 1; m is an integer selected from 1, or 2, and R¹⁸ is hydrogen or C₁₋₄alkyl; preferably R⁴⁰ is selected from the group comprising —[CHR¹³]ₘ—OH, —CO—C₁₋₂alkyl, —O—[CHR¹³]ₘ—O—C₁₋₂alkyl, m is an integer selected from 1, or 2, and R¹⁸ is hydrogen or C₁₋₂alkyl;
R⁴², R⁴³, R⁴⁴, R⁴⁵, R⁴⁶, R⁴⁷ are each independently selected from hydrogen or C₁₋₆alkyl; preferably R⁴², R⁴³, R⁴⁴, R⁴⁵, R⁴⁶, R⁴⁷ are each independently selected from hydrogen or C₁₋₄ alkyl; preferably R⁴², R⁴³, R⁴⁴, R⁴⁵, R⁴⁶, R⁴⁷ are each independently selected from hydrogen or C₁₋₂alkyl; preferably R⁴², R⁴³, R⁴⁴, R⁴⁵, R⁴⁶, R⁴⁷ are each independently selected from hydrogen or methyl;
or R⁴⁰ and R⁴² together with the carbon atom to which they are attached form a 3- to 6-membered heterocyclyl moiety; preferably or R⁴⁰ and R⁴² together with the carbon atom to which they are attached form a 3- to 5-membered heterocyclyl moiety; or R⁴⁰ and R⁴² together with the carbon atom to which they are attached form a 3- to 4-membered heterocyclyl moiety; or R⁴⁰ and R⁴² together with the carbon atom to which they are attached form a 3-membered heterocyclyl moiety, preferably a oxiranyl moiety.

17. The tanning composition according to any one of statements 1-2, wherein the compound is of formula (IVa),

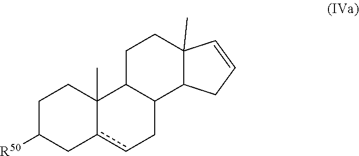

wherein R⁵⁰ is selected from OH or oxo.

18. The tanning composition according to any one of statements 1-17, wherein said compound is selected from the group comprising

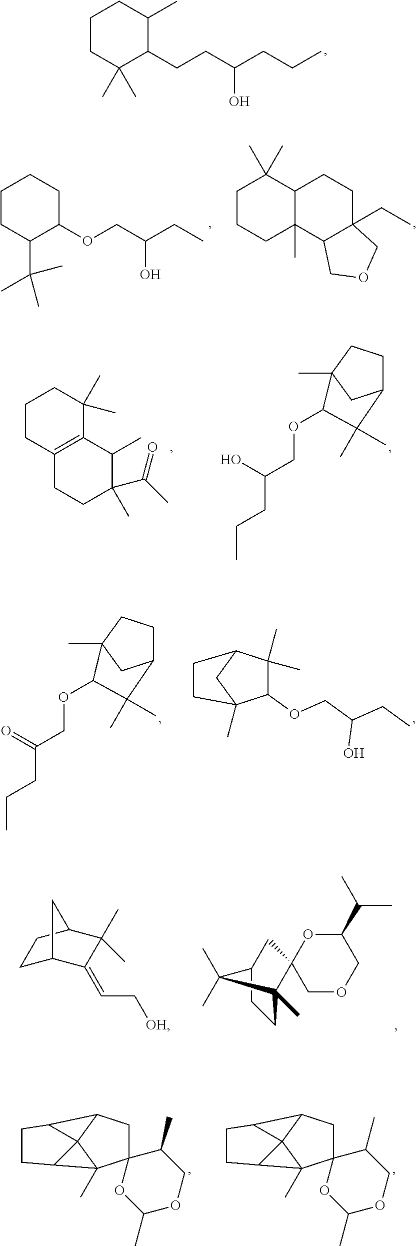

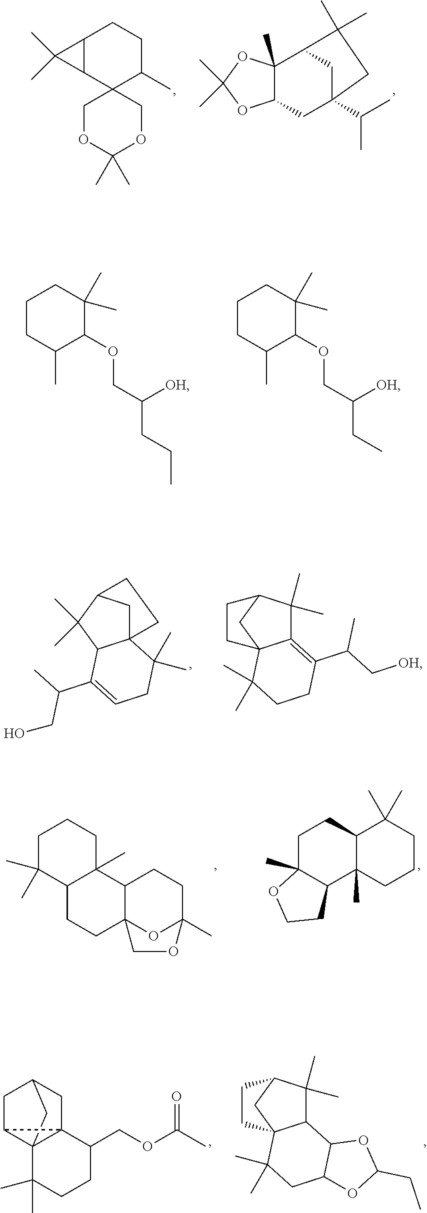

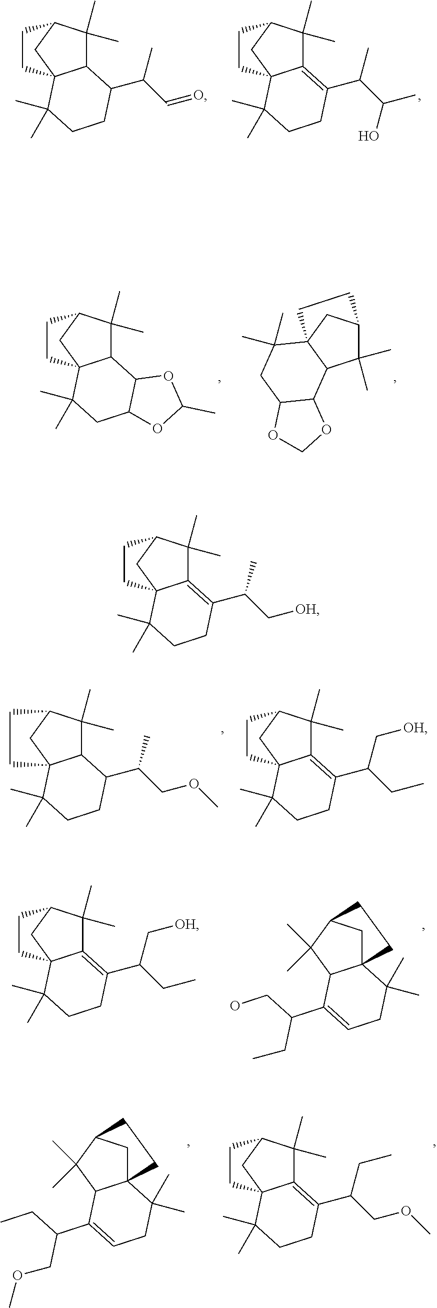

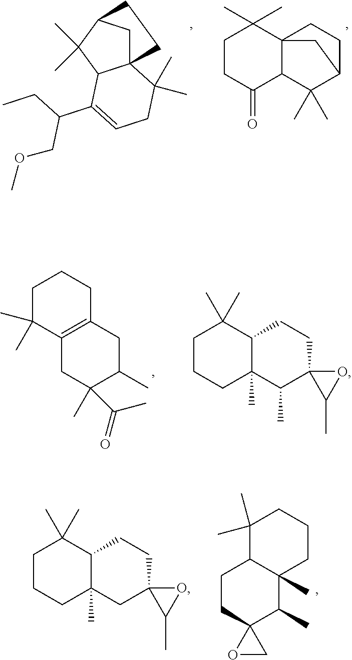

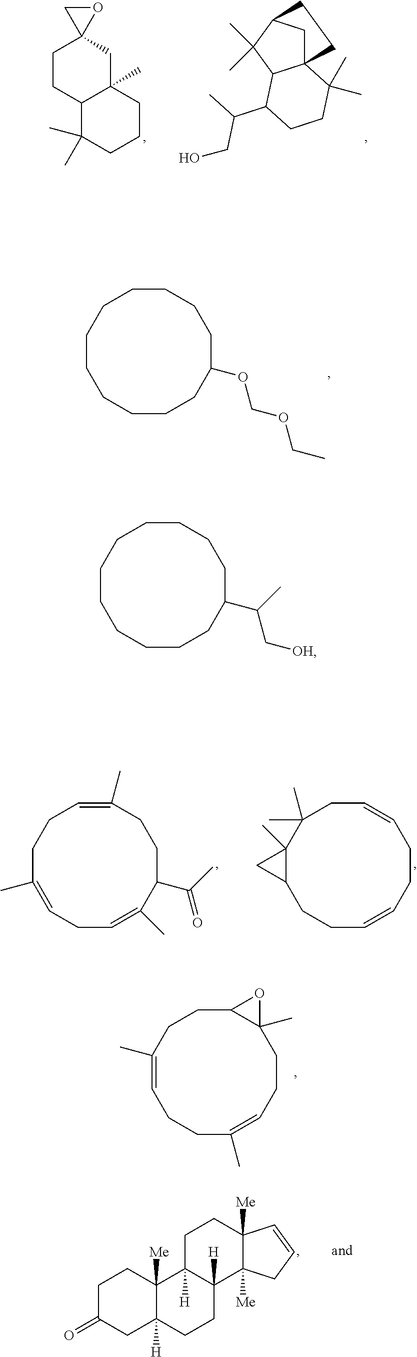

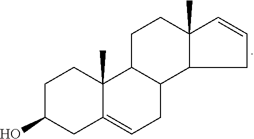 [SMILES: C[C@@]12C=CCC1C1CC=C3C[C@@H](O)CC[C@]3(C)C1CC2]

19. The tanning composition according to any one of statements 1-18, wherein said compound is Amberketal or Ambermax.

20. The tanning composition according to any one of statements 1-19, wherein said composition is a sunless tanning composition, a moisturizing composition or a haircare composition.

21. The tanning composition according to any one of statements 1-20, wherein said composition is selected from the group comprising a gel, solution, aerosol, emulsion, lotion, mousses, sprays, wipes, microencapsulation, serum, cream, ointment, powder and foam.

22. The tanning composition according to any one of statements 1-21, wherein said composition is incorporated in a device adapted for application under the skin tissue, to mucosal tissue, to the surface of skin or to the scalp.

23. The tanning composition according to any one of statements 1-22, further comprising a sunscreen agent, a skin conditioning agent, a tanning agent, a skin lightening agent, a skin protectant, an emollient, a thickening agent, a humectant, or any combination thereof.

24. The tanning composition according to any one of statements 1-23, wherein at least one further self-tanning substance is present.

25. A compound of formula (Ih), or a racemate, a stereoisomer, a tautomer, a salt, and/or a solvate thereof, including mixtures thereof,

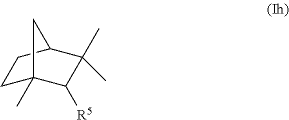

wherein
R⁵ is selected from the group comprising —O—CHR¹⁴—C(═O)—[CR¹⁷R¹⁸]ₘ—R¹⁹, —O—CHR¹⁴—CH(OH)—[CR¹⁷R¹⁸]ₘ—R¹⁹;
m is an integer selected from 1, 2, 3, 4 or 5; preferably m is selected from 1, 2, 3 or 4, preferably m is selected from 1, 2, or 3, preferably m is 1 or 2;
R¹⁴, R¹⁷, R¹⁸, R¹⁹ are each independently selected from hydrogen or C₁₋₆alkyl; preferably R¹⁴, R¹⁷, R¹⁸, R¹⁹ are each independently selected from hydrogen or C₁₋₄alkyl; preferably R¹⁴, R¹⁷, R¹⁸, R¹⁹ are each independently selected from hydrogen or C₁₋₂alkyl; preferably R¹⁴, R¹⁷, R¹⁸, R¹⁹ are each independently selected from hydrogen or methyl; preferably R¹⁴, R¹⁷, R¹⁸, are each independently hydrogen and R¹⁹ is methyl.

26. The compound according to statement 25 wherein said compound is selected from

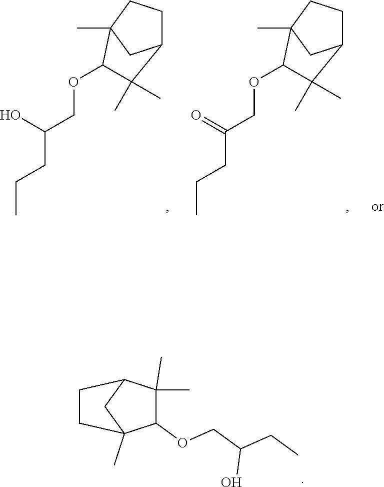

27. Use of a compound of formula (IIb), (IIa), (IIIa), (IV), (Ie), (If), (Ig), (Ic), or (Id), or a racemate, a stereoisomer, a tautomer, a salt, and/or a solvate thereof, including mixtures thereof, as self-tanning substance, preferably the non-therapeutic use of a compound of formula (IIb), (IIa), (IIIa), (IV), (Ie), (If), (Ig), (Ic), or (Id), or a racemate, a stereoisomer, a tautomer, a salt, and/or a solvate thereof, including mixtures thereof, as self-tanning substance,

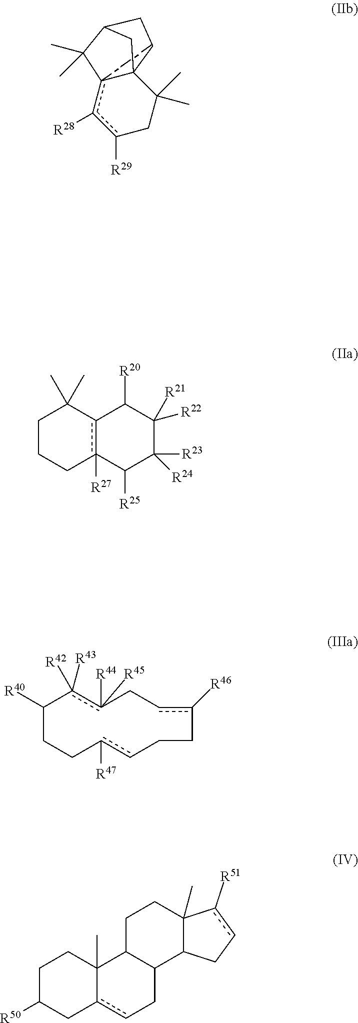

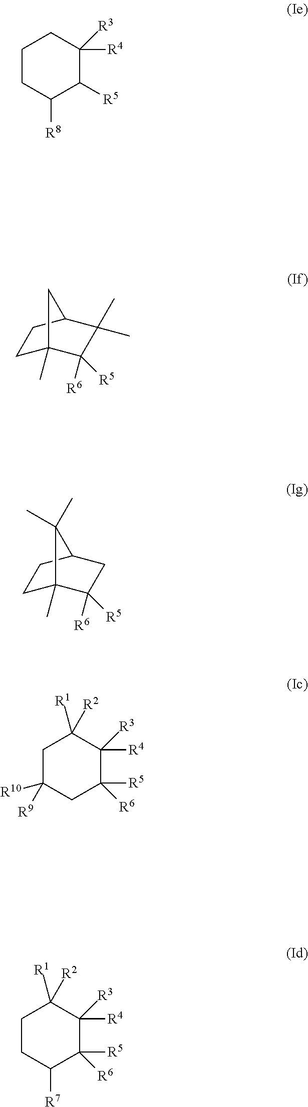

wherein the dash bond [symbol] represents an optional double bond; and wherein [symbol] represent an optional single bond;
R²⁸ is selected from the group comprising hydrogen, oxo, C₁₋₆alkyl, —O—C₁₋₆alkyl, —[CR¹⁷R¹⁸]ₘ—O—CO—C₁₋₆alkyl, —[CR¹⁷R¹⁸]ₘ—O—C₁₋₆alkyl, —[CR¹⁷R¹⁸]ₘ—COH, and —[CR¹⁷R¹⁸]ₘ—OH; m is an integer selected from 1, 2, 3, 4 or 5, R¹⁷, R¹⁸, are each independently selected from hydrogen or C₁₋₆alkyl;
R²⁹ is hydrogen or C₁₋₆alkyl;
or R²⁹ and R²⁸ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2;
R²⁰ is hydrogen or C₁₋₆alkyl;
R²¹ is hydrogen or C₁₋₆alkyl;
R²² is selected from the group comprising hydrogen, C₁₋₆alkyl, and —CO—R¹⁹;
R²³ is hydrogen or C₁₋₆alkyl;
R²⁴ is hydrogen or C₁₋₆alkyl;
or R²³ and R²⁴ together with the carbon atom to which they are attached form a 3- to 6-membered heterocyclyl moiety; wherein said heterocyclyl is optionally substituted with one or more substituents each independently selected from C₁₋₆alkyl;
R²⁵ is hydrogen or C₁₋₆alkyl;
or R²⁴ and R²⁵ form together with the carbon atom to which they are attached a 5 or 6-membered heterocyclyl moiety, wherein said heterocyclyl is optionally substituted with one or more substituents each independently selected from C₁₋₆alkyl, or wherein one of said substituent and R²³ form together with the atoms to which they are attached a 3- to 6-membered heterocyclyl moiety;
R²⁷ if present is hydrogen or C₁₋₆alkyl;
or R²⁰ and R²⁷ together with the carbon atoms to which they are attached form a C3-membered ring;
R⁴⁰ is selected from the group comprising —[CR¹⁷R¹⁸]ₘ—OH, —CO—C₁₋₆alkyl, —O—([CR¹⁷R¹⁸]ₘ—O—)ₜ—C₁₋₆alkyl, t is an integer selected from 0, 1, 2, or 3;
R⁴², R⁴³, R⁴⁴, R⁴⁵, R⁴⁶, R⁴⁷ are each independently selected from hydrogen or C₁₋₆alkyl; or R⁴⁰ and R⁴² together with the carbon atom to which they are attached form a 3- to 6-membered heterocyclyl moiety;
R⁵⁰ is selected from OH or oxo;
R⁵¹ is hydrogen or C₁₋₄alkyl;
R¹, R² are each independently selected from hydrogen or C₁₋₆alkyl;
R³, R⁴ are each independently selected from hydrogen or C₁₋₆alkyl;
or R¹ with R³ together with the carbon atoms to which they are attached form a saturated C3 to C5-membered ring, wherein said ring is optionally substituted with one or more C₁₋₆alkyl substituents;
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —X—[CR¹³R¹⁴]ₙ—O—[CR¹⁷R¹⁸]ₘ—OH, —([CR¹⁷R¹⁸]ₘ—O)_{y}—C₁₋₆alkyl, —[CR¹³R¹⁴]ₙ—CO—R¹⁹;
m is an integer selected from 1, 2, 3, 4 or 5, n is an integer selected from 0, 1, 2, 3 or 4; y is an integer selected from 1, 2 or 3;
X is a single bond or O,
R¹³, R¹⁴, R¹⁶, R¹⁷, R¹⁸, R¹⁹ are each independently selected from hydrogen or C₁₋₆alkyl;
R¹⁵ is OH, or R¹⁵ together with R¹⁶ form an oxo group;
or R³ and R⁵ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2;
R⁶ is hydrogen or C₁₋₆alkyl;
or R⁵ and R⁶ form together ═[CR¹⁴]—[CR¹⁷R¹⁸]ₘ—OH; or R⁵ and R⁶ together with the carbon atom to which they are attached form a 5- or 6-membered heterocyclyl moiety, wherein said heterocyclyl is optionally substituted with one or more C₁₋₆alkyl substituents;
R⁷ is hydrogen or C₁₋₆alkyl;
R⁸ is hydrogen or C₁₋₆alkyl;
or R⁵ and R⁸ together with the carbon atoms to which they are attached form a saturated or unsaturated C5 or C6-membered ring, wherein said ring is optionally substituted with one or more substituents each independently selected from oxo, C₁₋₆alkyl, —O—C₁₋₆alkyl, —CO—C₁₋₆alkyl, —[CR¹⁷R¹⁸]ₘ—O—CO—C₁₋₆alkyl, —([CR¹⁷R¹⁸]ₘ—O)_{y}—C₁₋₆alkyl, —[CR¹⁷R¹⁸]ₘ—COH, or —[CR¹⁷R¹⁸]ₘ—OH; or two substituents on said ring form together with the carbon atoms to which they are attached a 3- to 6-membered heterocyclyl moiety, wherein said heterocyclyl moiety is optionally substituted with one or more substituents each independently selected from C₁₋₆ alkyl, or two substituents on said heterocyclyl form together with the atoms to which they are attached a 3- to 6-membered heterocyclyl moiety;
R⁹, R¹⁰ are each independently selected from hydrogen or C₁₋₆alkyl;
or R⁶ and R⁹ together with the carbon atoms to which they are attached form a C3-membered ring;
or R¹ with R⁹ together with the carbon atoms to which they are attached form a saturated C5-membered ring, wherein said ring is optionally substituted with one or more C₁₋₆alkyl substituents;
or R³ with R⁹ together with the carbon atoms to which they are attached form a saturated C5-membered ring, wherein said ring is optionally substituted with one or more C₁₋₆alkyl substituents.

28. Use of a composition according to any one of statements 1-24, or use of a compound according to any one of statements 25-26, or the use according to statement 27, for darkening skin.

29. Use of a composition according to any one of statements 1-24, or use of a compound according to any one of statements 25-26, or the use according to statement 27, for sunless tanning.

30. Use of a composition according to any one of statements 1-24, or use of a compound according to any one of statements 25-26, or the use according to statement 27, for increasing the amount of melanin in melanocytes, for increasing melanin synthesis, for improving melanin transport and/or for improving the distribution of melanin in suprabasal layers.

31. Use of a compound of formula (I), (II), (III) or (IV) or a racemate, a stereoisomer, a tautomer, a salt, and/or a solvate thereof, including mixtures thereof, for increasing the amount of melanin in melanocytes, for increasing melanin synthesis, for improving melanin transport and/or for improving the distribution of melanin in suprabasal layers.

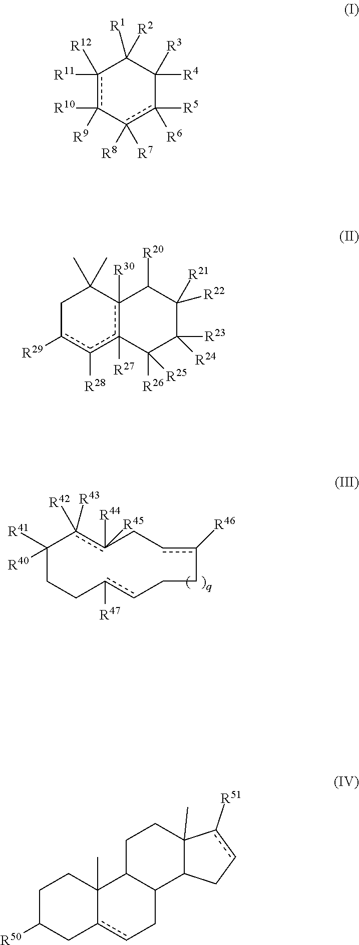

wherein the dash bond [symbol] represents an optional double bond; and wherein
R¹, R² are each independently selected from hydrogen or C₁₋₆alkyl;
R³, R⁴ are each independently selected from hydrogen or C₁₋₆alkyl;
or R¹ with R³ together with the carbon atoms to which they are attached form a saturated C3 to C5-membered ring, wherein said ring is optionally substituted with one or more C₁₋₆alkyl substituents;
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —X—[CR¹³R¹⁴]ₙ—O—[CR¹⁷R¹⁸]ₘ—OH, —([CR¹⁷R¹⁸]ₘ—O)_{y}—C₁₋₆alkyl, —[CR¹³R¹⁴]ₙ—CO—R¹⁹;
m is an integer selected from 1, 2, 3, 4 or 5, n is an integer selected from 0, 1, 2, 3 or 4; y is an integer selected from 1, 2 or 3;
X is a single bond or O,
R¹³, R¹⁴, R¹⁶, R¹⁷, R¹⁸, R¹⁹ are each independently selected from hydrogen or C₁₋₆alkyl;
R¹⁵ is OH, or R¹⁵ together with R¹⁶ form an oxo group;
or R³ and R⁵ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2;
or R⁵ and R⁶ form together ═[CR¹⁴]—[CR¹⁷R¹⁸]ₘ—OH; or R⁵ and R⁶ together with the carbon atom to which they are attached form a 5- or 6-membered heterocyclyl moiety, wherein said heterocyclyl is optionally substituted with one or more C₁₋₆alkyl substituents;
or R⁵ and R⁸ together with the carbon atoms to which they are attached form a saturated or unsaturated C5 or C6-membered ring, wherein said ring is optionally substituted with one or more substituents each independently selected from oxo, C₁₋₆alkyl, —O—C₁₋₆alkyl, —CO—C₁₋₆alkyl, —[CR¹⁷R¹⁸]ₘ—O—CO—C₁₋₆alkyl, —([CR¹⁷R¹⁸]ₘ—O)_{y}—C₁₋₆alkyl, —[CR¹⁷R¹⁸]ₘ—COH, or —[CR¹⁷R¹⁸]ₘ—OH; or two substituents on said ring form together with the carbon atoms to which they are attached a 3- to 6-membered heterocyclyl moiety, wherein said heterocyclyl moiety is optionally substituted with one or more substituents each independently selected from C₁₋₆ alkyl or oxo, or two substituents on said heterocyclyl form together with the atoms to which they are attached a 3- to 6-membered heterocyclyl moiety;
or R⁶ and R⁹ together with the carbon atoms to which they are attached form a C3-membered ring;
R⁶ if present is hydrogen or C₁₋₆alkyl;
R⁷ if present is hydrogen or C₁₋₆alkyl;
R⁸ is hydrogen or C₁₋₆alkyl;
or R¹ with R⁸ together with the carbon atoms to which they are attached form a saturated C5-membered ring, wherein said ring is optionally substituted with one or more C₁₋₆alkyl substituents;
R⁹, R¹⁰ are each independently selected from hydrogen or C₁₋₆alkyl;
or R¹ with R⁹ together with the carbon atoms to which they are attached form a saturated C5-membered ring, wherein said ring is optionally substituted with one or more C₁₋₆alkyl substituents;
or R³ with R⁹ together with the carbon atoms to which they are attached form a saturated C5-membered ring, wherein said ring is optionally substituted with one or more C₁₋₆alkyl substituents;
R¹¹, R¹² are each independently selected from hydrogen or C₁₋₆alkyl;
or R⁹ and R¹¹ together with the carbon atoms to which they are attached form a saturated or unsaturated C5 or C6-membered ring, wherein said ring is optionally substituted with one or more C₁₋₆alkyl substituents;
R²⁰ is hydrogen or C₁₋₆alkyl;
R²¹ is hydrogen or C₁₋₆alkyl;
R²² is selected from the group comprising hydrogen, C₁₋₆alkyl, and —CO—R¹⁹;
R²³ is hydrogen or C₁₋₆alkyl;
R²⁴ is hydrogen or C₁₋₆alkyl;
or R²³ and R²⁴ together with the carbon atom to which they are attached form a 3- to 6-membered heterocyclyl moiety; wherein said heterocyclyl is optionally substituted with one or more substituents each independently selected from C₁₋₆alkyl;
R²⁵ is hydrogen or C₁₋₆alkyl;
or R²⁴ and R²⁵ form together with the carbon atom to which they are attached a 5 or 6-membered heterocyclyl moiety, wherein said heterocyclyl is optionally substituted with one or more substituents each independently selected from C₁₋₆alkyl or oxo, or wherein one of said substituent and R²³ form together with the atoms to which they are attached a 3- to 6-membered heterocyclyl moiety;
R²⁶ is hydrogen or C₁₋₆alkyl;
R²⁷ if present is hydrogen or C₁₋₆alkyl;
or R²⁰ and R²⁷ together with the carbon atoms to which they are attached form a C3-membered ring;
R²⁸ is selected from the group comprising hydrogen, oxo, C₁₋₆alkyl, —O—C₁₋₆alkyl, —[CR¹⁷R¹⁸]ₘ—O—CO—C₁₋₆alkyl, —[CR¹⁷R¹⁸]ₘ—O—C₁₋₆alkyl, —[CR¹⁷R¹⁸]ₘ—COH, and —[CR¹⁷R¹⁸]ₘ—OH;
R²⁹ is hydrogen or C₁₋₆alkyl;
R³⁰ if present is hydrogen or C₁₋₆alkyl;
or R³⁰ with R²³ together with the carbon atoms to which they are attached form a saturated C3 to C5-membered ring, wherein said ring is optionally substituted with one or more C₁₋₆alkyl substituents;
or R²⁹ and R²⁸ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents;
q is an integer selected from 0, 1, 2, 3, preferably q is 1;
R⁴⁰ is selected from the group comprising —[CR¹⁷R¹⁸]ₘ—OH, —CO—C₁₋₆alkyl, —O—([CR¹⁷R¹⁸]ₘ—O—)ₜ—C₁₋₆alkyl, t is an integer selected from 0, 1, 2, or 3;
R⁴¹, R⁴², R⁴³, R⁴⁴, R⁴⁵, R⁴⁶, R⁴⁷ are each independently selected from hydrogen or C₁₋₆alkyl;
or R⁴⁰ and R⁴² together with the carbon atom to which they are attached form a 3- to 6-membered heterocyclyl moiety;
R⁵⁰ is selected from OH or oxo; and
R⁵¹ is hydrogen or C₁₋₁₀ alkyl.

32. Use of a compound of formula (I), (II), (III) or (IV) or a racemate, a stereoisomer, a tautomer, a salt, and/or a solvate thereof, including mixtures thereof, as defined in any one of statements 1-19, or use of a compound of formula (Ih) as defined in any one of statements 25-26, or the use according to statement 27, in the manufacture of a composition for increasing the amount of melanin in melanocytes, for increasing melanin synthesis, for improving melanin transport and/or for improving the distribution of melanin in suprabasal layers;

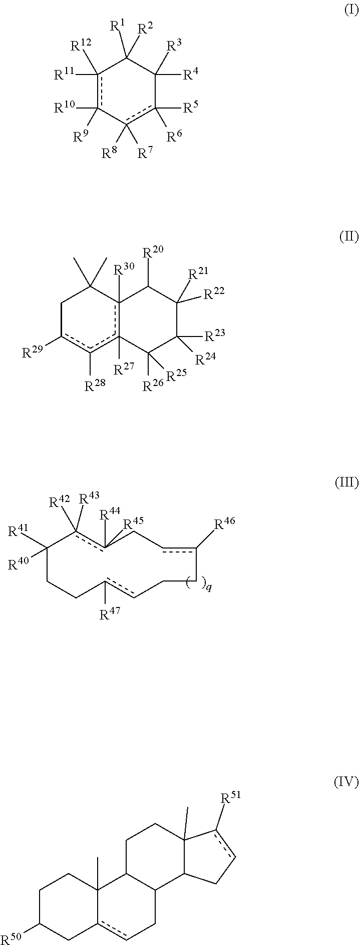

wherein the dash bond [symbol] represents an optional double bond; and wherein
R¹, R² are each independently selected from hydrogen or C₁₋₆alkyl;
R³, R⁴ are each independently selected from hydrogen or C₁₋₆alkyl;
or R¹ with R³ together with the carbon atoms to which they are attached form a saturated C3 to C5-membered ring, wherein said ring is optionally substituted with one or more C₁₋₆alkyl substituents;
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —X—[CR¹³R¹⁴]ₙ—O—[CR¹⁷R¹⁸]ₘ—OH, —([CR¹⁷R¹⁸]ₘ—O)_{y}—C₁₋₆alkyl, —[CR¹³R¹⁴]ₙ—CO—R¹⁹;
m is an integer selected from 1, 2, 3, 4 or 5, n is an integer selected from 0, 1, 2, 3 or 4; y is an integer selected from 1, 2 or 3;
X is a single bond or O,
R¹³, R¹⁴, R¹⁶, R¹⁷, R¹⁸, R¹⁹ are each independently selected from hydrogen or C₁₋₆alkyl;
R¹⁵ is OH, or R¹⁵ together with R¹⁶ form an oxo group;
or R³ and R⁵ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2;
or R⁵ and R⁶ form together ═[CR¹⁴]—[CR¹⁷R¹⁸]ₘ—OH; or R⁵ and R⁶ together with the carbon atom to which they are attached form a 5- or 6-membered heterocyclyl moiety, wherein said heterocyclyl is optionally substituted with one or more C₁₋₆alkyl substituents;
or R⁵ and R⁸ together with the carbon atoms to which they are attached form a saturated or unsaturated C5 or C6-membered ring, wherein said ring is optionally substituted with one or more substituents each independently selected from oxo, C₁₋₆alkyl, —O—C₁₋₆alkyl, —CO—C₁₋₆alkyl, —[CR¹⁷R¹⁸]ₘ—O—CO—C₁₋₆alkyl, —([CR¹⁷R¹⁸]ₘ—O)_{y}—C₁₋₆alkyl, —[CR¹⁷R¹⁸]ₘ—COH, or —[CR¹⁷R¹⁸]ₘ—OH; or two substituents on said ring form together with the carbon atoms to which they are attached a 3- to 6-membered heterocyclyl moiety, wherein said heterocyclyl moiety is optionally substituted with one or more substituents each independently selected from C₁₋₆ alkyl or oxo, or two substituents on said heterocyclyl form together with the atoms to which they are attached a 3- to 6-membered heterocyclyl moiety;
or R⁶ and R⁹ together with the carbon atoms to which they are attached form a C3-membered ring;
R⁶ if present is hydrogen or C₁₋₆alkyl;
R⁷ if present is hydrogen or C₁₋₆alkyl;
R⁸ is hydrogen or C₁₋₆alkyl;
or R¹ with R⁸ together with the carbon atoms to which they are attached form a saturated C5-membered ring, wherein said ring is optionally substituted with one or more C₁₋₆alkyl substituents;
R⁹, R¹⁰ are each independently selected from hydrogen or C₁₋₆alkyl;
or R¹ with R⁹ together with the carbon atoms to which they are attached form a saturated C5-membered ring, wherein said ring is optionally substituted with one or more C₁₋₆alkyl substituents;
or R³ with R⁹ together with the carbon atoms to which they are attached form a saturated C5-membered ring, wherein said ring is optionally substituted with one or more C₁₋₆alkyl substituents;
R¹¹, R¹² are each independently selected from hydrogen or C₁₋₆alkyl;
or R⁹ and R¹¹ together with the carbon atoms to which they are attached form a saturated or unsaturated C5 or C6-membered ring, wherein said ring is optionally substituted with one or more C₁₋₆alkyl substituents;
R²⁰ is hydrogen or C₁₋₆alkyl;
R²¹ is hydrogen or C₁₋₆alkyl;
R²² is selected from the group comprising hydrogen, C₁₋₆alkyl, and —CO—R¹⁹;
R²³ is hydrogen or C₁₋₆alkyl;
R²⁴ is hydrogen or C₁₋₆alkyl;
or R²³ and R²⁴ together with the carbon atom to which they are attached form a 3- to 6-membered heterocyclyl moiety; wherein said heterocyclyl is optionally substituted with one or more substituents each independently selected from C₁₋₆alkyl;
R²⁵ is hydrogen or C₁₋₆alkyl;
or R²⁴ and R²⁵ form together with the carbon atom to which they are attached a 5 or 6-membered heterocyclyl moiety, wherein said heterocyclyl is optionally substituted with one or more substituents each independently selected from C₁₋₆alkyl or oxo, or wherein one of said substituent and R²³ form together with the atoms to which they are attached a 3- to 6-membered heterocyclyl moiety;
R²⁶ is hydrogen or C₁₋₆alkyl;
R²⁷ if present is hydrogen or C₁₋₆alkyl;
or R²⁰ and R²⁷ together with the carbon atoms to which they are attached form a C3-membered ring;
R²⁸ is selected from the group comprising hydrogen, oxo, C₁₋₆alkyl, —O—C₁₋₆alkyl, —[CR¹⁷R¹⁸]ₘ—O—CO—C₁₋₆alkyl, —[CR¹⁷R¹⁸]ₘ—O—C₁₋₆alkyl, —[CR¹⁷R¹⁸]ₘ—COH, and —[CR¹⁷R¹⁸]ₘ—OH;
R²⁹ is hydrogen or C₁₋₆alkyl;
R³⁰ if present is hydrogen or C₁₋₆alkyl;
or R³⁰ with R²³ together with the carbon atoms to which they are attached form a saturated C3 to C5-membered ring, wherein said ring is optionally substituted with one or more C₁₋₆alkyl substituents;
or R²⁹ and R²⁸ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents;
q is an integer selected from 0, 1, 2, 3, preferably q is 1;
R⁴⁰ is selected from the group comprising —[CR¹⁷R¹⁸]ₘ—OH, —CO—C₁₋₆alkyl, —O—([CR¹⁷R¹⁸]ₘ—O—)ₜ—C₁₋₆alkyl, t is an integer selected from 0, 1, 2, or 3;
R⁴¹, R⁴², R⁴³, R⁴⁴, R⁴⁵, R⁴⁶, R⁴⁷ are each independently selected from hydrogen or C₁₋₆alkyl;
or R⁴⁰ and R⁴² together with the carbon atom to which they are attached form a 3- to 6-membered heterocyclyl moiety;
R⁵⁰ is selected from OH or oxo; and
R⁵¹ is hydrogen or C₁₋₁₀ alkyl.

33. A compound of formula (IIb), (IIa), (IIIa), (IV), (Ie), (If), (Ig), (Ic) or (Id), or a racemate, a stereoisomer, a tautomer, a salt, and/or a solvate thereof, including mixtures thereof, for use for increasing the amount of melanin in melanocytes, for increasing melanin synthesis, for improving melanin transport and/or for improving the distribution of melanin in suprabasal layers or a compound of formula (Ih) according to any one of statements 25, 26, for use for increasing the amount of melanin in melanocytes, for increasing melanin synthesis, for improving melanin transport and/or for improving the distribution of melanin in suprabasal layers,

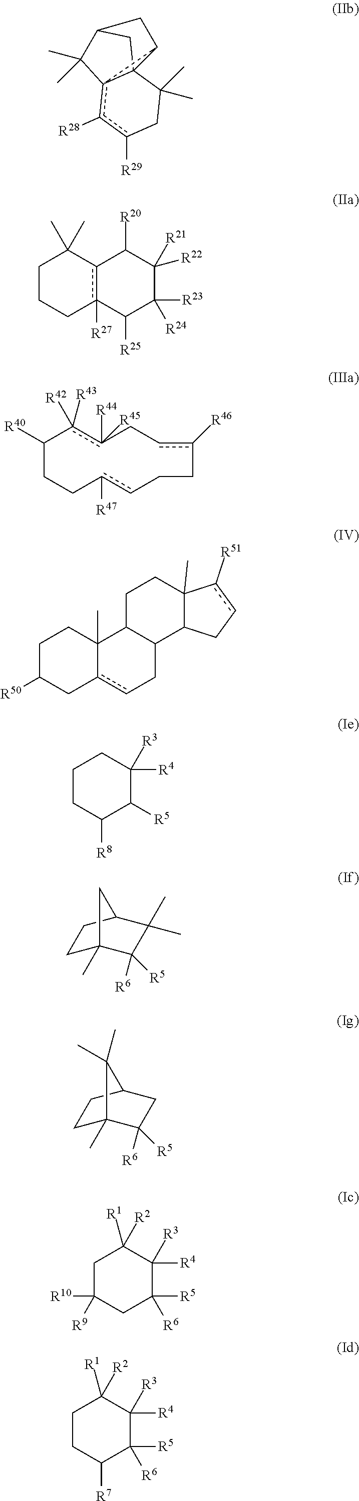

wherein the dash bond represents [symbol] an optional double bond; and wherein [symbol] represent an optional single bond;
R²⁸ is selected from the group comprising hydrogen, oxo, C₁₋₆alkyl, —O—C₁₋₆alkyl, —[CR¹⁷R¹⁸]ₘ—O—CO—C₁₋₆alkyl, —[CR¹⁷R¹⁸]ₘ—O—C₁₋₆alkyl, —[CR¹⁷R¹⁸]ₘ—COH, and —[CR¹⁷R¹⁸]ₘ—OH; m is an integer selected from 1, 2, 3, 4 or 5,
R¹⁷, R¹⁸, are each independently selected from hydrogen or C₁₋₆alkyl;
R²⁹ is hydrogen or C₁₋₆alkyl;
or R²⁹ and R²⁸ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2;
R²⁰ is hydrogen or C₁₋₆alkyl;
R²¹ is hydrogen or C₁₋₆alkyl;
R²² is selected from the group comprising hydrogen, C₁₋₆alkyl, and —CO—R¹⁹;
R²³ is hydrogen or C₁₋₆alkyl;
R²⁴ is hydrogen or C₁₋₆alkyl;
or R²³ and R²⁴ together with the carbon atom to which they are attached form a 3- to 6-membered heterocyclyl moiety; wherein said heterocyclyl is optionally substituted with one or more substituents each independently selected from C₁₋₆alkyl;
R²⁵ is hydrogen or C₁₋₆alkyl;
or R²⁴ and R²⁵ form together with the carbon atom to which they are attached a 5 or 6-membered heterocyclyl moiety, wherein said heterocyclyl is optionally substituted with one or more substituents each independently selected from C₁₋₆alkyl, or wherein one of said substituent and R²³ form together with the atoms to which they are attached a 3- to 6-membered heterocyclyl moiety;
R²⁷ if present is hydrogen or C₁₋₆alkyl;
or R²⁰ and R²⁷ together with the carbon atoms to which they are attached form a C3-membered ring;
R⁴⁰ is selected from the group comprising —[CR¹⁷R¹⁸]ₘ—OH, —CO—C₁₋₆alkyl, —O—([CR¹⁷R¹⁸]ₘ—O—)ₜ—C₁₋₆alkyl, t is an integer selected from 0, 1, 2, or 3;
R⁴², R⁴³, R⁴⁴, R⁴⁵, R⁴⁶, R⁴⁷ are each independently selected from hydrogen or C₁₋₆alkyl;
or R⁴⁰ and R⁴² together with the carbon atom to which they are attached form a 3- to 6-membered heterocyclyl moiety;
R⁵⁰ is selected from OH or oxo;
R⁵¹ is hydrogen or C₁₋₄alkyl;
R¹, R² are each independently selected from hydrogen or C₁₋₆alkyl;
R³, R⁴ are each independently selected from hydrogen or C₁₋₆alkyl;
or R¹ with R³ together with the carbon atoms to which they are attached form a saturated C3 to C5-membered ring, wherein said ring is optionally substituted with one or more C₁₋₆alkyl substituents;
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —X—[CR¹³R¹⁴]ₙ—O—[CR¹⁷R¹⁸]ₘ—OH, —([CR¹⁷R¹⁸]ₘ—O)_{y}—C₁₋₆alkyl, —[CR¹³R¹⁴]ₙ—CO—R¹⁹;
m is an integer selected from 1, 2, 3, 4 or 5, n is an integer selected from 0, 1, 2, 3 or 4; y is an integer selected from 1, 2 or 3;
X is a single bond or O,
R¹³, R¹⁴, R¹⁶, R¹⁷, R¹⁸, R¹⁹ are each independently selected from hydrogen or C₁₋₆alkyl;
R¹⁵ is OH, or R¹⁵ together with R¹⁶ form an oxo group;
or R³ and R⁵ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2;
R⁶ is hydrogen or C₁₋₆alkyl;
R⁷ is hydrogen or C₁₋₆alkyl;
R⁸ is hydrogen or C₁₋₆alkyl;
or R⁵ and R⁶ form together ═[CR¹⁴]—[CR¹⁷R¹⁸]ₘ—OH; or R⁵ and R⁶ together with the carbon atom to which they are attached form a 5- or 6-membered heterocyclyl moiety, wherein said heterocyclyl is optionally substituted with one or more C₁₋₆alkyl substituents;
or R⁵ and R⁸ together with the carbon atoms to which they are attached form a saturated or unsaturated C5 or C6-membered ring, wherein said ring is optionally substituted with one or more substituents each independently selected from oxo, C₁₋₆alkyl, —O—C₁₋₆alkyl, —CO—C₁₋₆alkyl, —[CR¹⁷R¹⁸]ₘ—O—CO—C₁₋₆alkyl, —([CR¹⁷R¹⁸]ₘ—O)_{y}—C₁₋₆alkyl, —[CR¹⁷R¹⁸]ₘ—COH, or —[CR¹⁷R¹⁸]ₘ—OH; or two substituents on said ring form together with the carbon atoms to which they are attached a 3- to 6-membered heterocyclyl moiety, wherein said heterocyclyl moiety is optionally substituted with one or more substituents each independently selected from C₁₋₆ alkyl, or two substituents on said heterocyclyl form together with the atoms to which they are attached a 3- to 6-membered heterocyclyl moiety;
R⁹, R¹⁰ are each independently selected from hydrogen or C₁₋₆alkyl;
or R⁶ and R⁹ together with the carbon atoms to which they are attached form a C3-membered ring;
or R¹ with R⁹ together with the carbon atoms to which they are attached form a saturated C5-membered ring, wherein said ring is optionally substituted with one or more C₁₋₆alkyl substituents;
or R³ with R⁹ together with the carbon atoms to which they are attached form a saturated C5-membered ring, wherein said ring is optionally substituted with one or more C₁₋₆alkyl substituents.

34. The use according to any one of statements 30-32, or the compound for use according to statement 33, wherein said melanocytes are present in human skin or hair.

35. The use according to any one of statements 30-32, or the compound for use according to statement 33, wherein the melanin is pheomelanin, eumelanin or any combination thereof.

36. Use of a compound of formula (I), (II), (III) or (IV) or a racemate, a stereoisomer, a tautomer, a salt, and/or a solvate thereof, including mixtures thereof, or use of a compound according to any one of statements 1-26, as self-tanning substance;

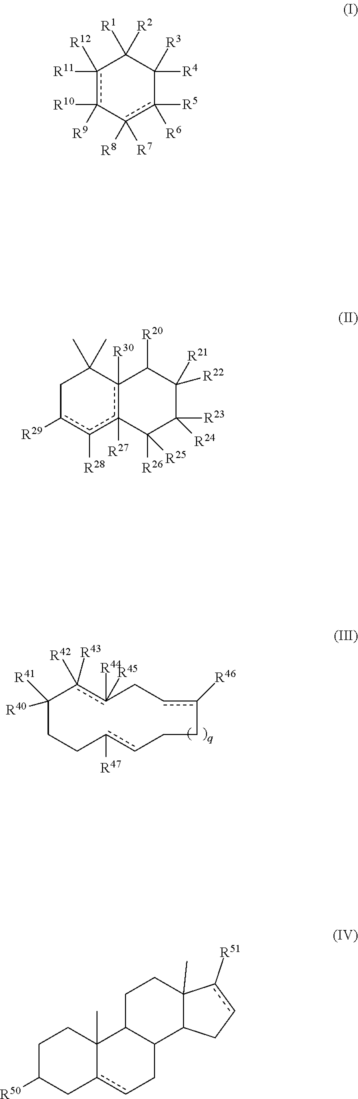

wherein the dash bond [symbol] represents an optional double bond; and wherein
R¹, R² are each independently selected from hydrogen or C₁₋₆alkyl;
R³, R⁴ are each independently selected from hydrogen or C₁₋₆alkyl;
or R¹ with R³ together with the carbon atoms to which they are attached form a saturated C3 to C5-membered ring, wherein said ring is optionally substituted with one or more C₁₋₆alkyl substituents;
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —X—[CR¹³R¹⁴]ₙ—O—[CR¹⁷R¹⁸]ₘ—OH, —([CR¹⁷R¹⁸]ₘ—O)_{y}—C₁₋₆alkyl, —[CR¹³R¹⁴]ₙ—CO—R¹⁹;
m is an integer selected from 1, 2, 3, 4 or 5, n is an integer selected from 0, 1, 2, 3 or 4; y is an integer selected from 1, 2 or 3;
X is a single bond or O,
R¹³, R¹⁴, R¹⁶, R¹⁷, R¹⁸, R¹⁹ are each independently selected from hydrogen or C₁₋₆alkyl;
R¹⁵ is OH, or R¹⁵ together with R¹⁶ form an oxo group;
or R³ and R⁵ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2;
or R⁵ and R⁶ form together ═[CR¹⁴]—[CR¹⁷R¹⁸]ₘ—OH; or R⁵ and R⁶ together with the carbon atom to which they are attached form a 5- or 6-membered heterocyclyl moiety, wherein said heterocyclyl is optionally substituted with one or more C₁₋₆alkyl substituents;
or R⁵ and R⁸ together with the carbon atoms to which they are attached form a saturated or unsaturated C5 or C6-membered ring, wherein said ring is optionally substituted with one or more substituents each independently selected from oxo, C₁₋₆alkyl, —O—C₁₋₆alkyl, —CO—C₁₋₆alkyl, —[CR¹⁷R¹⁸]ₘ—O—CO—C₁₋₆alkyl, —([CR¹⁷R¹⁸]ₘ—O)_{y}—C₁₋₆alkyl, —[CR¹⁷R¹⁸]ₘ—COH, or —[CR¹⁷R¹⁸]ₘ—OH; or two substituents on said ring form together with the carbon atoms to which they are attached a 3- to 6-membered heterocyclyl moiety, wherein said heterocyclyl moiety is optionally substituted with one or more substituents each independently selected from C₁₋₆ alkyl or oxo, or two substituents on said heterocyclyl form together with the atoms to which they are attached a 3- to 6-membered heterocyclyl moiety;
or R⁶ and R⁹ together with the carbon atoms to which they are attached form a C3-membered ring;
R⁶ if present is hydrogen or C₁₋₆alkyl;
R⁷ if present is hydrogen or C₁₋₆alkyl;
R⁸ is hydrogen or C₁₋₆alkyl;
or R¹ with R⁸ together with the carbon atoms to which they are attached form a saturated C5-membered ring, wherein said ring is optionally substituted with one or more C₁₋₆alkyl substituents;
R⁹, R¹⁰ are each independently selected from hydrogen or C₁₋₆alkyl;
or R¹ with R⁹ together with the carbon atoms to which they are attached form a saturated C5-membered ring, wherein said ring is optionally substituted with one or more C₁₋₆alkyl substituents;
or R³ with R⁹ together with the carbon atoms to which they are attached form a saturated C5-membered ring, wherein said ring is optionally substituted with one or more C₁₋₆alkyl substituents;
R¹¹, R¹² are each independently selected from hydrogen or C₁₋₆alkyl;
or R⁹ and R¹¹ together with the carbon atoms to which they are attached form a saturated or unsaturated C5 or C6-membered ring, wherein said ring is optionally substituted with one or more C₁₋₆alkyl substituents;
R²⁰ is hydrogen or C₁₋₆alkyl;
R²¹ is hydrogen or C₁₋₆alkyl;
R²² is selected from the group comprising hydrogen, C₁₋₆alkyl, and —CO—R¹⁹;
R²³ is hydrogen or C₁₋₆alkyl;
R²⁴ is hydrogen or C₁₋₆alkyl;
or R²³ and R²⁴ together with the carbon atom to which they are attached form a 3- to 6-membered heterocyclyl moiety; wherein said heterocyclyl is optionally substituted with one or more substituents each independently selected from C₁₋₆alkyl;
R²⁵ is hydrogen or C₁₋₆alkyl;
or R²⁴ and R²⁵ form together with the carbon atom to which they are attached a 5 or 6-membered heterocyclyl moiety, wherein said heterocyclyl is optionally substituted with one or more substituents each independently selected from C₁₋₆alkyl or oxo, or wherein one of said substituent and R²³ form together with the atoms to which they are attached a 3- to 6-membered heterocyclyl moiety;
R²⁶ is hydrogen or C₁₋₆alkyl;
R²⁷ if present is hydrogen or C₁₋₆alkyl;
or R²⁰ and R²⁷ together with the carbon atoms to which they are attached form a C3-membered ring;
R²⁸ is selected from the group comprising hydrogen, oxo, C₁₋₆alkyl, —O—C₁₋₆alkyl, —[CR¹⁷R¹⁸]ₘ—O—CO—C₁₋₆alkyl, —[CR¹⁷R¹⁸]ₘ—O—C₁₋₆alkyl, —[CR¹⁷R¹⁸]ₘ—COH, and —[CR¹⁷R¹⁸]ₘ—OH;
R²⁹ is hydrogen or C₁₋₆alkyl;
R³⁰ if present is hydrogen or C₁₋₆alkyl;
or R³⁰ with R²³ together with the carbon atoms to which they are attached form a saturated C3 to C5-membered ring, wherein said ring is optionally substituted with one or more C₁₋₆alkyl substituents;
or R²⁹ and R²⁸ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents;
q is an integer selected from 0, 1, 2, 3, preferably q is 1;
R⁴⁰ is selected from the group comprising —[CR¹⁷R¹⁸]ₘ—OH, —CO—C₁₋₆alkyl, —O—([CR¹⁷R¹⁸]ₘ—O—)ₜ—C₁₋₆alkyl, t is an integer selected from 0, 1, 2, or 3;
R⁴¹, R⁴², R⁴³, R⁴⁴, R⁴⁵, R⁴⁶, R⁴⁷ are each independently selected from hydrogen or C₁₋₆alkyl;
or R⁴⁰ and R⁴² together with the carbon atom to which they are attached form a 3- to 6-membered heterocyclyl moiety;
R⁵⁰ is selected from OH or oxo; and
R⁵¹ is hydrogen or C₁₋₁₀ alkyl.

37. The use according to any one of statements 27-29, wherein the compound is of formula (Ia), (Ib), (Ic), (Id), (IIa), (IIIa), or (IVa),

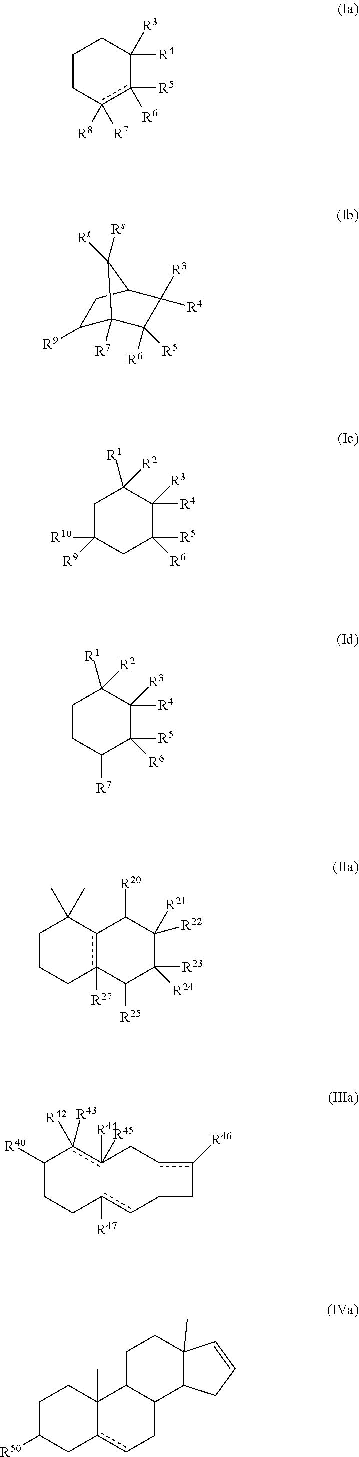

wherein R¹, R², R³, R⁴, R⁵, R⁶, R⁷, R⁸, R⁹, R¹⁰, R²⁰, R²¹, R²², R²³, R²⁴, R²⁵, R²⁷, R⁴⁰, R⁴², R⁴³, R⁴⁴, R⁴⁵, R⁴⁶, R⁴⁷, R⁵⁰ are as defined in any one of statements 1, 22, 23, 26, and R^{t} and R^{s} are each independently selected from hydrogen or C₁₋₆alkyl.

38. Non-therapeutic use of a compound of formula (IIb), (IIa), (IIIa), (IV), (Ie), (If), (Ig), (Ic), or (Id) or a racemate, a stereoisomer, a tautomer, a salt, and/or a solvate thereof, including mixtures thereof, as self-tanning substance,

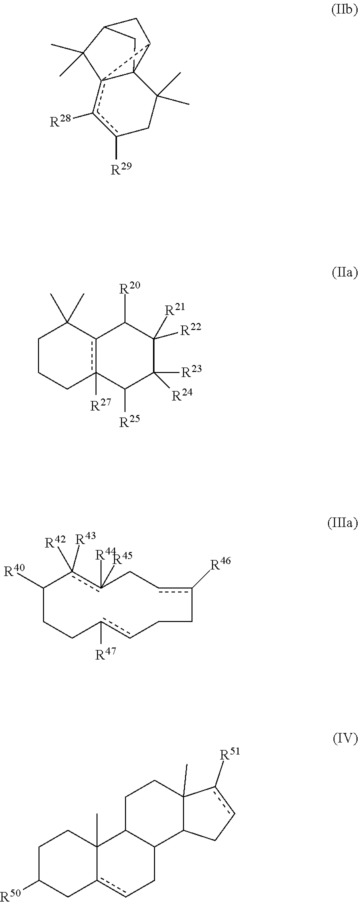

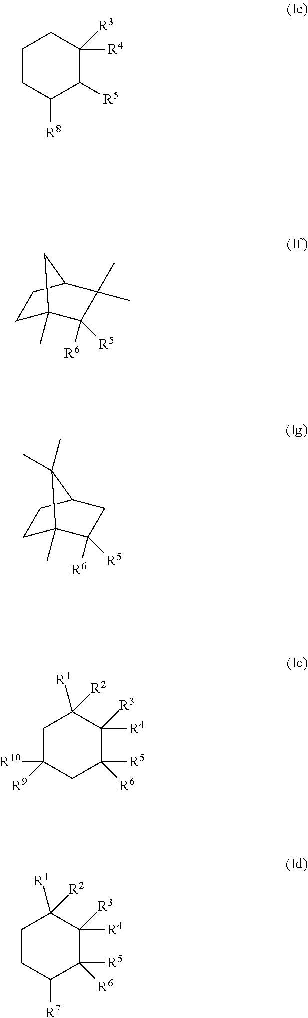

wherein the dash bond [symbol] represents an optional double bond; and wherein [symbol] represent an optional single bond;
R²⁸ is selected from the group comprising hydrogen, oxo, C₁₋₆alkyl, —O—C₁₋₆alkyl, —[CR¹⁷R¹⁸]ₘ—O—CO—C₁₋₆alkyl, —[CR¹⁷R¹⁸]ₘ—O—C₁₋₆alkyl, —[CR¹⁷R¹⁸]ₘ—COH, and —[CR¹⁷R¹⁸]ₘ—OH; m is an integer selected from 1, 2, 3, 4 or 5,
R¹⁷, R¹⁸, are each independently selected from hydrogen or C₁₋₆alkyl;
R²⁹ is hydrogen or C₁₋₆alkyl;
or R²⁹ and R²⁸ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2;
R²⁰ is hydrogen or C₁₋₆alkyl;
R²¹ is hydrogen or C₁₋₆alkyl;
R²² is selected from the group comprising hydrogen, C₁₋₆alkyl, and —CO—R¹⁹;
R²³ is hydrogen or C₁₋₆alkyl;
R²⁴ is hydrogen or C₁₋₆alkyl;
or R²³ and R²⁴ together with the carbon atom to which they are attached form a 3- to 6-membered heterocyclyl moiety; wherein said heterocyclyl is optionally substituted with one or more substituents each independently selected from C₁₋₆alkyl;
R²⁵ is hydrogen or C₁₋₆alkyl;
or R²⁴ and R²⁵ form together with the carbon atom to which they are attached a 5 or 6-membered heterocyclyl moiety, wherein said heterocyclyl is optionally substituted with one or more substituents each independently selected from C₁₋₆alkyl, or wherein one of said substituent and R²³ form together with the atoms to which they are attached a 3- to 6-membered heterocyclyl moiety;
R²⁷ if present is hydrogen or C₁₋₆alkyl;
or R²⁰ and R²⁷ together with the carbon atoms to which they are attached form a C3-membered ring;
R⁴⁰ is selected from the group comprising —[CR¹⁷R¹⁸]ₘ—OH, —CO—C₁₋₆alkyl, —O—([CR¹⁷R¹⁸]ₘ—O—)ₜ—C₁₋₆alkyl, t is an integer selected from 0, 1, 2, or 3;
R⁴², R⁴³, R⁴⁴, R⁴⁵, R⁴⁶, R⁴⁷ are each independently selected from hydrogen or C₁₋₆alkyl;
or R⁴⁰ and R⁴² together with the carbon atom to which they are attached form a 3- to 6-membered heterocyclyl moiety;
R⁵⁰ is selected from OH or oxo;
R⁵¹ is hydrogen or C₁₋₄alkyl;
R¹, R² are each independently selected from hydrogen or C₁₋₆alkyl;
R³, R⁴ are each independently selected from hydrogen or C₁₋₆alkyl;
or R¹ with R³ together with the carbon atoms to which they are attached form a saturated C3 to C5-membered ring, wherein said ring is optionally substituted with one or more C₁₋₆alkyl substituents;
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —X—[CR¹³R¹⁴]ₙ—O—[CR¹⁷R¹⁸]ₘ—OH, —([CR¹⁷R¹⁸]ₘ—O)_{y}—C₁₋₆alkyl, —[CR¹³R¹⁴]ₙ—CO—R¹⁹;
m is an integer selected from 1, 2, 3, 4 or 5, n is an integer selected from 0, 1, 2, 3 or 4; y is an integer selected from 1, 2 or 3;
X is a single bond or O,
R¹³, R¹⁴, R¹⁶, R¹⁷, R¹⁸, R¹⁹ are each independently selected from hydrogen or C₁₋₆alkyl;
R¹⁵ is OH, or R¹⁵ together with R¹⁶ form an oxo group;
or R³ and R⁵ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2;
R⁶ is hydrogen or C₁₋₆alkyl;
or R⁵ and R⁶ form together ═[CR¹⁴]—[CR¹⁷R¹⁸]ₘ—OH; or R⁵ and R⁶ together with the carbon atom to which they are attached form a 5- or 6-membered heterocyclyl moiety, wherein said heterocyclyl is optionally substituted with one or more C₁₋₆alkyl substituents;
R⁷ is hydrogen or C₁₋₆alkyl;
R⁸ is hydrogen or C₁₋₆alkyl;
or R⁵ and R⁸ together with the carbon atoms to which they are attached form a saturated or unsaturated C5 or C6-membered ring, wherein said ring is optionally substituted with one or more substituents each independently selected from oxo, C₁₋₆alkyl, —O—C₁₋₆alkyl, —CO—C₁₋₆alkyl, —[CR¹⁷R¹⁸]ₘ—O—CO—C₁₋₆alkyl, —([CR¹⁷R¹⁸]ₘ—O)_{y}—C₁₋₆alkyl, —[CR¹⁷R¹⁸]ₘ—COH, or —[CR¹⁷R¹⁸]ₘ—OH; or two substituents on said ring form together with the carbon atoms to which they are attached a 3- to 6-membered heterocyclyl moiety, wherein said heterocyclyl moiety is optionally substituted with one or more substituents each independently selected from C₁₋₆ alkyl, or two substituents on said heterocyclyl form together with the atoms to which they are attached a 3- to 6-membered heterocyclyl moiety;
R⁹, R¹⁰ are each independently selected from hydrogen or C₁₋₆alkyl;
or R⁶ and R⁹ together with the carbon atoms to which they are attached form a C3-membered ring;
or R¹ with R⁹ together with the carbon atoms to which they are attached form a saturated C5-membered ring, wherein said ring is optionally substituted with one or more C₁₋₆alkyl substituents;
or R³ with R⁹ together with the carbon atoms to which they are attached form a saturated C5-membered ring, wherein said ring is optionally substituted with one or more C₁₋₆alkyl substituents.

39. The use according to any one of statements 27-32, 34-38, or the compound for use according to statement 33, wherein the compound is of formula (Ie),

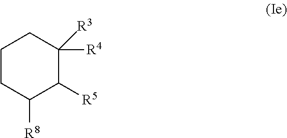

wherein
R³, R⁴ are each independently selected from hydrogen or C₁₋₆alkyl;
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]n-[CR¹⁷R¹⁸]ₘ—OH, —CO—R¹⁹;
m is an integer selected from 1, 2, 3, 4 or 5, n is an integer selected from 0, 1, 2, 3 or 4;
X is a single bond or O,
R¹³, R¹⁴, R¹⁶, R¹⁷, R¹⁸, R¹⁹ are each independently selected from hydrogen or C₁₋₆alkyl;
R¹⁵ is OH, or R¹⁵ together with R¹⁶ form an oxo group; and
R⁸ is hydrogen or C₁₋₆alkyl.

40. The use according to any one of statements 27-32, 34-38, or the compound for use according to statement 33, wherein the compound is of formula (If),

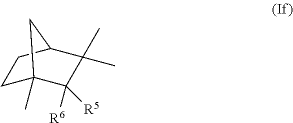

wherein
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —CO—R¹⁹;
m is an integer selected from 1, 2, 3, 4 or 5, n is an integer selected from 0, 1, 2, 3 or 4;
X is a single bond or O,
R¹³, R¹⁴, R¹⁶, R¹⁷, R¹⁸, R¹⁹ are each independently selected from hydrogen or C₁₋₆alkyl;
R¹⁵ is OH, or R¹⁵ together with R¹⁶ form an oxo group; and
R⁶ is hydrogen or C₁₋₆alkyl;
or R⁵ and R⁶ form together ═[CR¹⁴]—[CR¹⁷R¹⁸]ₘ—OH.

41. The use according to any one of statements 27-32, 34-38, or the compound for use according to statement 33, wherein the compound is of formula (Ig),

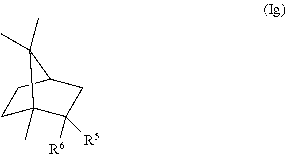

wherein
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —CO—R¹⁹;
m is an integer selected from 1, 2, 3, 4 or 5, n is an integer selected from 0, 1, 2, 3 or 4;
X is a single bond or O,
R¹³, R¹⁴, R¹⁶, R¹⁷, R¹⁸, R¹⁹ are each independently selected from hydrogen or C₁₋₆alkyl;
R¹⁵ is OH, or R¹⁵ together with R¹⁶ form an oxo group;
R⁶ is hydrogen or C₁₋₆alkyl;
or R⁵ and R⁶ form together ═[CR¹⁴]—[CR¹⁷R¹⁸]ₘ—OH; or R⁵ and R⁶ together with the carbon atom to which they are attached form a 5- or 6-membered heterocyclyl moiety, wherein said heterocyclyl is optionally substituted with one or more C₁₋₆alkyl substituents.

42. The use according to any one of statements 27-32, 34-38, or the compound for use according to statement 33, wherein the compound is of formula (IIb),

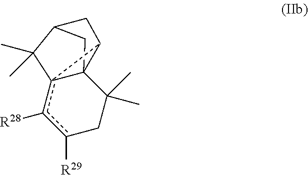

wherein the dash bond [symbol] represents an optional double bond; and wherein [symbol] represent an optional single bond;
R²⁸ is selected from the group comprising hydrogen, oxo, C₁₋₆alkyl, —O—C₁₋₆alkyl, —[CR¹⁷R¹⁸]ₘ—O—CO—C₁₋₆alkyl, —[CR¹⁷R¹⁸]ₘ—O—C₁₋₆alkyl, —[CR¹⁷R¹⁸]ₘ—COH, and —[CR¹⁷R¹⁸]ₘ—OH; wherein R²⁹ is hydrogen or C₁₋₆alkyl;
or R²⁹ and R²⁸ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents;
R¹⁷, R¹⁸, are each independently selected from hydrogen or C₁₋₆alkyl;
m is an integer selected from 1, 2, 3, 4 or 5; and p is an integer selected from 1 or 2.

43. The use according to any one of statements 27-32, 34-38, or the compound for use according to statement 33, wherein the compound is of formula (IIa),

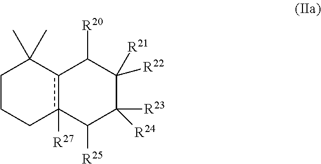

wherein the dash bond [symbol] represents an optional double bond; and wherein
R²⁰ is hydrogen or C₁₋₆alkyl;
R²¹ is hydrogen or C₁₋₆alkyl;
R²² is selected from the group comprising hydrogen, C₁₋₆alkyl, and —CO—R¹⁹;
R²³ is hydrogen or C₁₋₆alkyl;
R²⁴ is hydrogen or C₁₋₆alkyl;
or R²³ and R²⁴ together with the carbon atom to which they are attached form a 3-membered heterocyclyl moiety; wherein said heterocyclyl is optionally substituted with one or two substituents each independently selected from C₁₋₆alkyl;
R²⁵ is hydrogen or C₁₋₆alkyl;
or R²⁴ and R²⁵ form together with the carbon atom to which they are attached a 5 or 6-membered heterocyclyl moiety, wherein said heterocyclyl is optionally substituted with one or more substituents each independently selected from C₁₋₆alkyl, or wherein one of said substituent and R²³ form together with the atoms to which they are attached a 5- to 6-membered heterocyclyl moiety;
R²⁷ if present is hydrogen or C₁₋₆alkyl.

44. The use according to any one of statements 27-32, 34-38, 43, or the compound for use according to statement 33, wherein the compound is of formula (IIa),
wherein the dash bond [symbol] represents an optional double bond; and wherein
R²⁰ is hydrogen or C₁₋₆alkyl; preferably R²⁰ is hydrogen or C₁₋₄alkyl; preferably R²⁰ is hydrogen or C₁₋₂alkyl; preferably R²⁰ is hydrogen or methyl;
R²¹ is hydrogen or C₁₋₆alkyl; preferably R²¹ is hydrogen or C₁₋₄alkyl; preferably R²¹ is hydrogen or C₁₋₂alkyl; preferably R²¹ is hydrogen or methyl;
R²² is selected from the group comprising hydrogen, C₁₋₆alkyl, and —CO—R¹⁹; with R¹⁹ being hydrogen or C₁₋₆alkyl; preferably R²² is selected from the group comprising hydrogen, C₁₋₄alkyl, and —CO—R¹⁹; with R¹⁹ being hydrogen or C₁₋₄alkyl; preferably R²² is selected from the group comprising hydrogen, C₁₋₂alkyl, and —CO—R¹⁹; with R¹⁹ being hydrogen or C₁₋₂alkyl; preferably R²² is selected from the group comprising hydrogen, methyl, and —CO-methyl;
R²³ is hydrogen or C₁₋₆alkyl; preferably R²³ is hydrogen or C₁₋₄alkyl; preferably R²³ is hydrogen, methyl or ethyl;
R²⁴ is hydrogen or C₁₋₆alkyl; preferably R²⁴ is hydrogen or C₁₋₄alkyl; preferably R²⁴ is hydrogen or C₁₋₂alkyl; preferably R²⁴ is hydrogen or methyl;
or R²³ and R²⁴ together with the carbon atom to which they are attached form a 3- to 6-membered heterocyclyl moiety containing one or two O atoms, the rest being carbon atoms; wherein said heterocyclyl is optionally substituted with one or more substituents each independently selected from C₁₋₆alkyl; preferably R²³ and R²⁴ together with the carbon atom to which they are attached form a 3- to 5-membered heterocyclyl moiety containing one or two O atoms, the rest being carbon atoms; wherein said heterocyclyl is optionally substituted with one or more C₁₋₄alkyl substituents; preferably R²³ and R²⁴ together with the carbon atom to which they are attached form a 3- to 4-membered heterocyclyl moiety containing one O atom, the rest being carbon atoms; wherein said heterocyclyl is optionally substituted with one or more C₁₋₂alkyl substituents; preferably R²³ and R²⁴ together with the carbon atom to which they are attached form an oxiranyl; wherein said oxiranyl is optionally substituted with one or more C₁₋₂alkyl substituents, preferably methyl;
R²⁵ is hydrogen or C₁₋₆alkyl; preferably R²⁵ is hydrogen or C₁₋₄alkyl; preferably R²⁵ is hydrogen or C₁₋₂alkyl; preferably R²⁵ is hydrogen or methyl;
or R²⁴ and R²⁵ form together with the carbon atom to which they are attached a 5 or 6-membered heterocyclyl moiety containing one or two O atoms, the rest being carbon atoms; wherein said heterocyclyl is optionally substituted with one or more substituents each independently selected from C₁₋₆alkyl or oxo, or wherein one of said substituent and R²³ form together with the atoms to which they are attached a 3- to 6-membered heterocyclyl moiety containing one or two O atoms, the rest being carbon atoms; preferably R²⁴ and R²⁵ form together with the carbon atom to which they are attached a 5 or 6-membered heterocyclyl moiety containing one or two O atoms, the rest being carbon atoms, wherein said heterocyclyl is optionally substituted with one or more substituents each independently selected from C₁₋₄ alkyl or oxo, or wherein one of said substituent and R²³ form together with the atoms to which they are attached a 4- to 6-membered heterocyclyl moiety containing one or two O atoms, the rest being carbon atoms; preferably R²⁴ and R²⁵ together with the carbon atom to which they are attached form a tetrahydropyranyl or a tetrahydrofuranyl, wherein said tetrahydropyranyl or a tetrahydrofuranyl is optionally substituted with one or more substituents each independently selected from C₁₋₂alkyl or oxo, or wherein one of said substituent and R²³ form together with the atoms to which they are attached a 5- to 6-membered heterocyclyl moiety containing one or two O atoms, the rest being carbon atoms; such as a dioxolanyl;
R²⁷ if present is hydrogen or C₁₋₆alkyl; preferably R²⁷ is hydrogen or C₁₋₄alkyl; preferably R²⁷ is hydrogen or C₁₋₂alkyl; preferably R²⁷ is hydrogen or methyl.

45. The use according to any one of statements 27-32, 34-38, 43-44, or the compound for use according to statement 33, wherein the compound is of formula (IIa),
wherein the dash bond [symbol] represents an optional double bond; and wherein
R²⁰ is hydrogen or C₁₋₄alkyl; preferably R²⁰ is hydrogen or C₁₋₂alkyl; preferably R²⁰ is hydrogen or methyl;
R²¹ is hydrogen or C₁₋₄alkyl; preferably R²¹ is hydrogen or C₁₋₂alkyl; preferably R²¹ is hydrogen or methyl;
R²² is selected from the group comprising hydrogen, C₁₋₄alkyl, and —CO—R¹⁹; with R¹⁹ being hydrogen or C₁₋₄alkyl; preferably R²² is selected from the group comprising hydrogen, C₁₋₂alkyl, and —CO—R¹⁹; with R¹⁹ being hydrogen or C₁₋₂alkyl; preferably R²² is selected from the group comprising hydrogen, methyl, and —CO-methyl;
R²³ is hydrogen or C₁₋₄alkyl; preferably R²³ is hydrogen, methyl or ethyl;
R²⁴ is hydrogen or C₁₋₄alkyl; preferably R²⁴ is hydrogen or C₁₋₂alkyl; preferably R²⁴ is hydrogen or methyl;
or R²³ and R²⁴ together with the carbon atom to which they are attached form a 3- to 5-membered heterocyclyl moiety containing one or two O atoms, the rest being carbon atoms; wherein said heterocyclyl is optionally substituted with one or more C₁₋₄alkyl substituents; preferably R²³ and R²⁴ together with the carbon atom to which they are attached form a 3- to 4-membered heterocyclyl moiety containing one O atom, the rest being carbon atoms; wherein said heterocyclyl is optionally substituted with one or more C₁₋₂alkyl substituents; preferably R²³ and R²⁴ together with the carbon atom to which they are attached form an oxiranyl; wherein said oxiranyl is optionally substituted with one or more C₁₋₂alkyl substituents, preferably methyl;
R²⁵ is hydrogen or C₁₋₄alkyl; preferably R²⁵ is hydrogen or C₁₋₂alkyl; preferably R²⁵ is hydrogen or methyl;
or R²⁴ and R²⁵ form together with the carbon atom to which they are attached a 5 or 6-membered heterocyclyl moiety containing one or two O atoms, the rest being carbon atoms, wherein said heterocyclyl is optionally substituted with one or more substituents each independently selected from C₁₋₄alkyl or oxo, or wherein one of said substituent and R²³ form together with the atoms to which they are attached a 4- to 6-membered heterocyclyl moiety containing one or two O atoms, the rest being carbon atoms; preferably R²⁴ and R²⁵ together with the carbon atom to which they are attached form a tetrahydropyranyl or a tetrahydrofuranyl, wherein said tetrahydropyranyl or a tetrahydrofuranyl is optionally substituted with one or more substituents each independently selected from C₁₋₂alkyl or oxo, or wherein one of said substituent and R²³ form together with the atoms to which they are attached a 5- to 6-membered heterocyclyl moiety containing one or two O atoms, the rest being carbon atoms; such as a dioxolanyl;
R²⁷ is hydrogen or C₁₋₄alkyl; preferably R²⁷ is hydrogen or C₁₋₂alkyl; preferably R²⁷ is hydrogen or methyl.

46. The use according to any one of statements 27-32, 34-38, or the compound for use according to statement 33, wherein the compound is of formula (Ic),

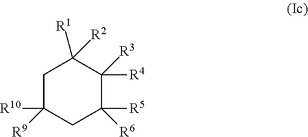

wherein R¹, R² are each independently selected from hydrogen or C₁₋₆alkyl;
R³, R⁴ are each independently selected from hydrogen or C₁₋₆alkyl;
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —CO—R¹⁹;
m is an integer selected from 1, 2, 3, 4 or 5, n is an integer selected from 0, 1, 2, 3 or 4;
X is a single bond or O,
R¹³, R¹⁴, R¹⁶, R¹⁷, R¹⁸, R¹⁹ are each independently selected from hydrogen or C₁₋₆alkyl;
R¹⁵ is OH, or R¹⁵ together with R¹⁶ form an oxo group;
or R³ and R⁵ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2;
R⁶ is hydrogen or C₁₋₆alkyl;
R⁹, R¹⁰ are each independently selected from hydrogen or C₁₋₆alkyl;
or R¹ with R⁹ together with the carbon atoms to which they are attached form a saturated C5-membered ring, wherein said ring is optionally substituted with one or more C₁₋₆alkyl substituents.

47. The use according to any one of statements 27-32, 34-38, 46, or the compound for use according to statement 33, wherein the compound is of formula (Ic), wherein
R² is hydrogen or C₁₋₆alkyl; preferably R² is hydrogen or C₁₋₄alkyl; preferably R² is hydrogen or C₁₋₂alkyl; preferably R² is hydrogen or methyl, preferably hydrogen;
R⁴ is hydrogen or C₁₋₆alkyl; preferably R⁴ is hydrogen or C₁₋₄alkyl; preferably R⁴ is hydrogen or C₁₋₂alkyl; preferably R⁴ is hydrogen or methyl;
R³ and R⁵ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2, R¹⁷, R¹⁸, are each independently selected from hydrogen or C₁₋₆alkyl; preferably R³ and R⁵ together are of formula —O—[CR¹⁷R¹⁸]—O—, and form together with the carbon atoms to which they are attached a 5-membered heterocyclyl moiety, wherein said heterocyclyl is optionally substituted with one or more C₁₋₆alkyl substituents; preferably R³ and R⁵ together are of formula —O—[CR¹⁷R¹⁸]—O—, and form together with the carbon atoms to which they are attached a dioxolanyl moiety, R¹⁷, R¹⁸, are each independently selected from hydrogen or C₁₋₄alkyl, wherein said dioxolanyl moiety is optionally substituted with one or two C₁₋₄alkyl substituents, preferably one or two C₁₋₂alkyl substituents, preferably one or two methyl;
R⁶ is hydrogen or C₁₋₆alkyl; preferably R⁶ is hydrogen or C₁₋₄alkyl; preferably R⁶ is hydrogen or C₁₋₂alkyl; preferably R⁶ is hydrogen or methyl;
R¹⁰ is hydrogen or C₁₋₆alkyl; preferably R¹⁰ is hydrogen or C₁₋₄alkyl; R¹⁰ is hydrogen or C₁₋₃alkyl, preferably hydrogen, methyl, ethyl, n-propyl or isopropyl;
and R¹ with R⁹ together with the carbon atoms to which they are attached form a saturated C5-membered ring, wherein said ring is optionally substituted with one or more C₁₋₆alkyl substituents; preferably wherein said ring is optionally substituted with one or more C₁₋₄alkyl substituents; preferably wherein said ring is optionally substituted with one or more C₁₋₂alkyl substituents, preferably wherein said ring is optionally substituted with one or more methyl substituents.

48. The use according to any one of statements 27-32, 34-38, 46-47, or the compound for use according to statement 33, wherein the compound is of formula (Ic), wherein
R² is hydrogen or C₁₋₄alkyl; preferably R² is hydrogen or C₁₋₂alkyl; preferably R² is hydrogen or methyl, preferably hydrogen;
R⁴ is hydrogen or C₁₋₄alkyl; preferably R⁴ is hydrogen or C₁₋₂alkyl; preferably R⁴ is hydrogen or methyl;
R³ and R⁵ together are of formula —O—[CR¹⁷R¹⁸]—O—, and form together with the carbon atoms to which they are attached a 5-membered heterocyclyl moiety, wherein said heterocyclyl is optionally substituted with one or more C₁₋₆alkyl substituents; preferably R³ and R⁵ together are of formula —O—[CR¹⁷R¹⁸]—O—, and form together with the carbon atoms to which they are attached a dioxolanyl moiety, R¹⁷, R¹⁸, are each independently selected from hydrogen or C₁₋₄ alkyl, wherein said dioxolanyl moiety is optionally substituted with one or two C₁₋₄alkyl substituents, preferably one or two C₁₋₂alkyl substituents, preferably one or two methyl;
R⁶ is hydrogen or C₁₋₄alkyl; preferably R⁶ is hydrogen or C₁₋₂alkyl; preferably R⁶ is hydrogen or methyl;
R¹⁰ is hydrogen or C₁₋₄alkyl; R¹⁰ is hydrogen or C₁₋₃alkyl, preferably hydrogen, methyl, ethyl, n-propyl or isopropyl;
and R¹ with R⁹ together with the carbon atoms to which they are attached form a saturated C5-membered ring, wherein said ring is optionally substituted with one or more C₁₋₆alkyl substituents; preferably wherein said ring is optionally substituted with one or more C₁₋₄alkyl substituents; preferably wherein said ring is optionally substituted with one or more C₁₋₂alkyl substituents, preferably wherein said ring is optionally substituted with one or more methyl substituents.

49. The use according to any one of statements 27-32, 34-38, or the compound for use according to statement 33, wherein the compound is of formula (Id),

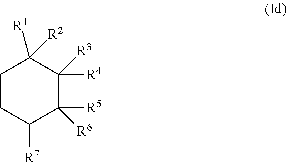

wherein R¹, R² are each independently selected from hydrogen or C₁₋₆alkyl;
R³, R⁴ are each independently selected from hydrogen or C₁₋₆alkyl;
or R¹ with R³ together with the carbon atoms to which they are attached form a saturated C3 to C5-membered ring, wherein said ring is optionally substituted with one or more C₁₋₆alkyl substituents;
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —CO—R¹⁹;
m is an integer selected from 1, 2, 3, 4 or 5, n is an integer selected from 0, 1, 2, 3 or 4;
X is a single bond or O,
R¹³, R¹⁴, R¹⁶, R¹⁷, R¹⁸, R¹⁹ are each independently selected from hydrogen or C₁₋₆alkyl;
R¹⁵ is OH, or R¹⁵ together with R¹⁶ form an oxo group;
R⁶ is hydrogen or C₁₋₆alkyl;
or R⁵ and R⁶ form together ═[CR¹⁴]—[CR¹⁷R¹⁸]ₘ—OH;
or R⁵ and R⁶ together with the carbon atom to which they are attached form a 5- or 6-membered heterocyclyl moiety, wherein said heterocyclyl is optionally substituted with one or more C₁₋₆ alkyl substituents;
R⁷ is hydrogen or C₁₋₆alkyl.

50. The use according to any one of statements 27-32, 34-38, 49, or the compound for use according to statement 33, wherein the compound is of formula (Id), wherein
R² is hydrogen or C₁₋₆alkyl; preferably R² is hydrogen or C₁₋₄alkyl; preferably R² is hydrogen or C₁₋₂alkyl; preferably R² is hydrogen or methyl, preferably hydrogen;
R⁴ is hydrogen or C₁₋₆alkyl; preferably R⁴ is hydrogen or C₁₋₄alkyl; preferably R⁴ is hydrogen or C₁₋₂alkyl; preferably R⁴ is hydrogen or methyl, preferably hydrogen;
R¹ with R³ together with the carbon atoms to which they are attached form a saturated C3 to C5-membered ring, wherein said ring is optionally substituted with one or more C₁₋₆alkyl substituents; preferably R¹ with R³ together with the carbon atoms to which they are attached form a C3 to C4-membered ring, wherein said ring is optionally substituted with one or more C₁₋₄alkyl substituents; preferably R¹ with R³ together with the carbon atoms to which they are attached form a C3-membered ring i.e. a cyclopropyl, wherein said cyclopropyl is optionally substituted with one or more C₁₋₂alkyl substituents; preferably wherein said cyclopropyl is optionally substituted with one or more methyl substituents;
R⁶ is hydrogen or C₁₋₆alkyl; preferably R⁶ is hydrogen or C₁₋₄alkyl; preferably R⁶ is hydrogen or C₁₋₂alkyl; preferably R⁶ is hydrogen or methyl;
R⁵ and R⁶ together with the carbon atom to which they are attached form a 5- or 6-membered heterocyclyl moiety, wherein said heterocyclyl is optionally substituted with one or more C₁₋₆ alkyl substituents; preferably R⁵ and R⁶ together with the carbon atom to which they are attached form a 6-membered heterocyclyl moiety, wherein said heterocyclyl is optionally substituted with one or more C₁₋₆alkyl substituents; preferably R⁵ and R⁶ together with the carbon atom to which they are attached form a 6-membered dioxanyl moiety, for example 1,3-dioxanyl, wherein said dioxanyl is optionally substituted with one or more C₁₋₆alkyl substituents, for example one or more C₁₋₄alkyl substituents, for example one or more C₁₋₃ alkyl substituents, for example one or more C₁₋₂alkyl substituents, for example one or more methyl substituents; and
R⁷ is hydrogen or C₁₋₆alkyl, preferably R⁷ is hydrogen or C₁₋₄alkyl; preferably R⁷ is hydrogen or C₁₋₂alkyl; preferably R⁷ is hydrogen or methyl, preferably methyl.

51. The use according to any one of statements 27-32, 34-38, 49-50, or the compound for use according to statement 33, wherein the compound is of formula (Id), wherein
R² is hydrogen or C₁₋₄alkyl; preferably R² is hydrogen or C₁₋₂alkyl; preferably R² is hydrogen or methyl, preferably hydrogen;
R⁴ is hydrogen or C₁₋₄alkyl; preferably R⁴ is hydrogen or C₁₋₂alkyl; preferably R⁴ is hydrogen or methyl, preferably hydrogen;
R¹ with R³ together with the carbon atoms to which they are attached form a C3 to C4-membered ring, wherein said ring is optionally substituted with one or more C₁₋₄alkyl substituents; preferably R¹ with R³ together with the carbon atoms to which they are attached form a C3-membered ring i.e. a cyclopropyl, wherein said cyclopropyl is optionally substituted with one or more C₁₋₂alkyl substituents; preferably wherein said cyclopropyl is optionally substituted with one or more methyl substituents;
R⁶ is hydrogen or C₁₋₄alkyl; preferably R⁶ is hydrogen or C₁₋₂alkyl; preferably R⁶ is hydrogen or methyl;
R⁵ and R⁶ together with the carbon atom to which they are attached form a 6-membered heterocyclyl moiety, wherein said heterocyclyl is optionally substituted with one or more C₁₋₆ alkyl substituents; preferably R⁵ and R⁶ together with the carbon atom to which they are attached form a 6-membered dioxanyl moiety, for example 1,3-dioxanyl, wherein said dioxanyl is optionally substituted with one or more C₁₋₆alkyl substituents, for example one or more C₁₋₄ alkyl substituents, for example one or more C₁₋₃alkyl substituents, for example one or more C₁₋₂alkyl substituents, for example one or more methyl substituents; and
R⁷ is hydrogen or C₁₋₄alkyl; preferably R⁷ is hydrogen or C₁₋₂alkyl; preferably R⁷ is hydrogen or methyl, preferably methyl.

52. The use according to any one of statements 27-32, 34-38, or the compound for use according to statement 33, wherein the compound is of formula (IIIa),

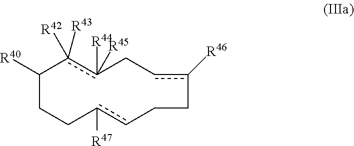

wherein the dash bond [symbol] represents an optional double bond; and wherein
R⁴⁰ is selected from the group comprising —[CR¹⁷R¹³]ₘ—OH, —CO—C₁₋₆alkyl, —O—([CR¹⁷R¹⁸]ₘ—O—)ₜ—C₁₋₆alkyl, m is an integer selected from 1, 2, 3, 4 or 5, t is an integer selected from 0, or 1; R¹⁷, R¹³, are each independently selected from hydrogen or C₁₋₆alkyl;
R⁴², R⁴³, R⁴⁴, R⁴⁵, R⁴⁶, R⁴⁷ are each independently selected from hydrogen or C₁₋₆alkyl;
or R⁴⁰ and R⁴² together with the carbon atom to which they are attached form a 3-membered heterocyclyl moiety.

53. The use according to any one of statements 27-32, 34-38, or the compound for use according to statement 33, wherein the compound is of formula (IVa),

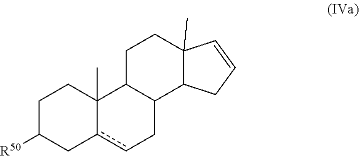

wherein R⁵⁰ is selected from OH, or oxo.

54. The use according to any one of statements 27-32, 34-38, or the compound for use according to statement 33, wherein said compound is selected from the group comprising

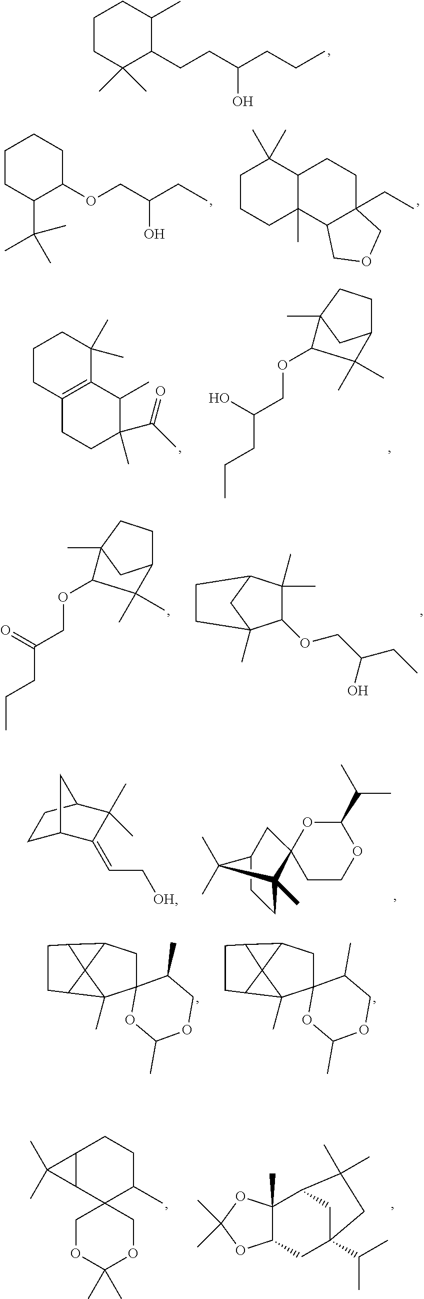

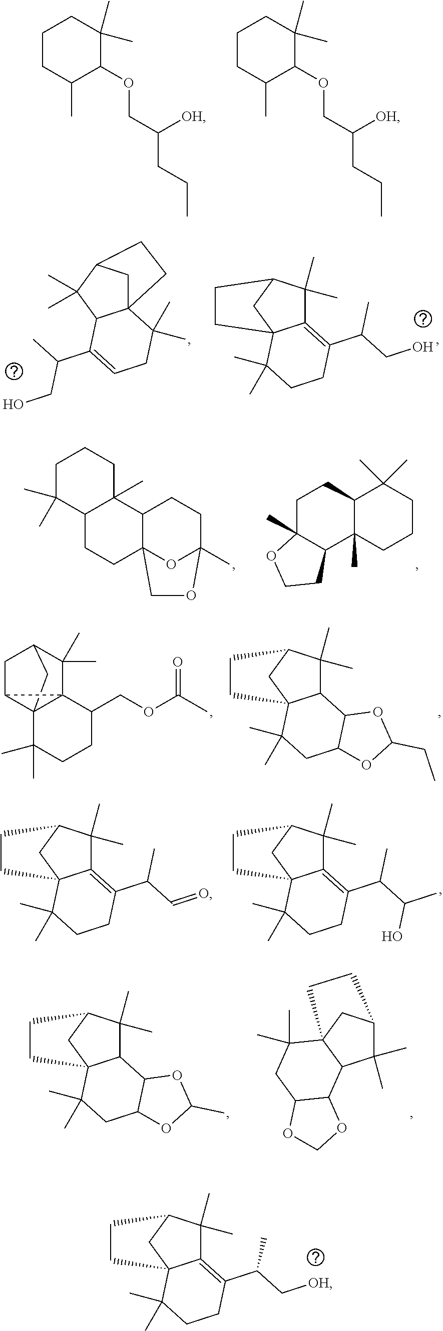

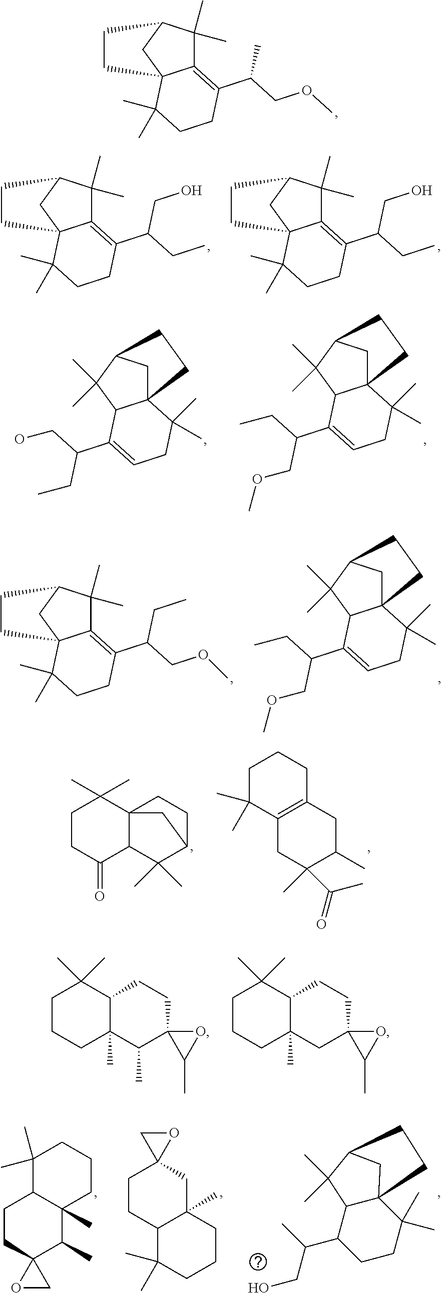

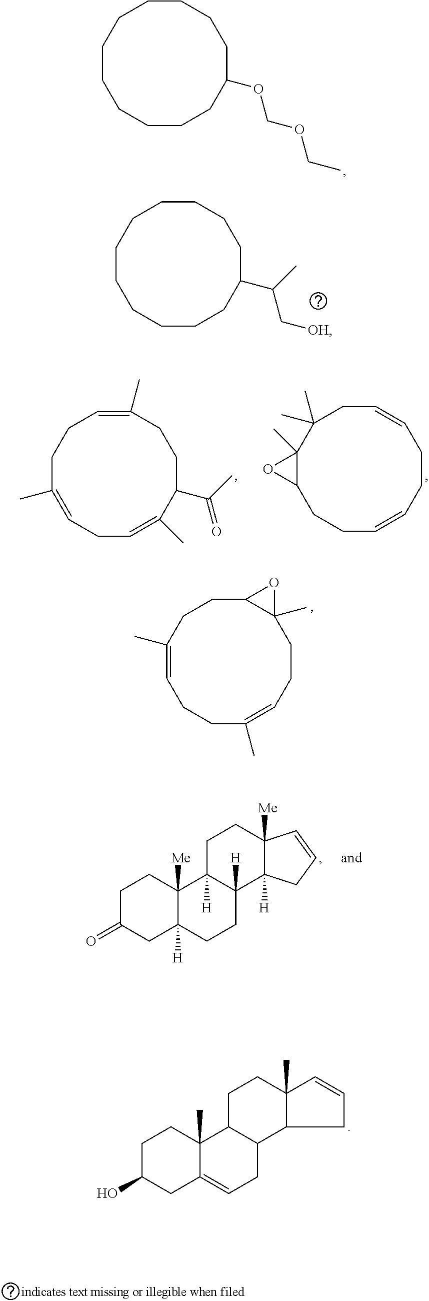

55. The use according to any one of statements 27-32, 34-38, or the compound for use according to statement 33, wherein said compound is Amberketal or Ambermax.

56. The use according to any one of statements 27-32, 34-55, wherein said use is in a cosmetic composition.

57. The use according to any one of statements 27-32, 34-56, or the compound for use according to statement 33, comprising modulating hair color by contacting a hair follicle with said compound or said composition, thereby increasing the concentration of pigment in new hair growth produced by the hair follicle, preferably wherein the hair follicle is part of the scalp, eyebrow, or eyelash region.

58. The use according to any one of statements 27-32, 34-57, wherein said use is for non-therapeutic purpose.

59. The use according to any one of statements 27-32, 34-58, wherein said composition is a sunless tanning composition, a moisturizing composition or a haircare composition.

60. The use according to any one of statements 27-32, 34-59, wherein said composition can be a gel, solution, aerosol, emulsion, lotion, mousses, sprays, wipes, microencapsulation, serum, cream, ointment, powder or foam.

61. The use according to any one of statements 27-32, 34-60, wherein said composition is incorporated in a device adapted for application under the skin tissue, to mucosal tissue, to the surface of skin or to the scalp.

62. The use according to any one of statements 27-32, 34-61, for darkening skin.

63. The use according to any one of statements 27-32, 34-62, for sunless tanning.

64. A tanning method comprising using a composition according to any one of statements 1-24, or a compound according to any one of statements 25-26, or using one of more compounds of the formula (I), (II), (III), (IV), (IIb), (IIa), (IIIa), (Ie), (If), (Ig), (Ic), or (Id), or a racemate, a stereoisomer, a tautomer, a salt, and/or a solvate thereof, including mixtures thereof, according to any one of statements 27-32, 34-55, as a self-tanning substance.

65. A self-tanning method comprising the step of applying onto a desired skin surface a self-tanning substance selected from the group comprising a compound as defined in any one of statements 1-32, 34-55, or a racemate, a stereoisomer, a tautomer, a salt, and/or a solvate thereof, including mixtures thereof.

66. A method for increasing the amount of melanin in melanocytes, for increasing melanin synthesis, for improving melanin transport and/or for improving the distribution of melanin in suprabasal layers comprising topically applying a composition according to any one of statements 1-24, or applying a compound according to any one of statements 25-26, or applying one of more compounds of the formula (I), (II), (III), (IV), (IIb), (IIa), (IIIa), (Ie), (If), (Ig), (Ic), or (Id), or a racemate, a stereoisomer, a tautomer, a salt, and/or a solvate thereof, including mixtures thereof, according to any one of statements 27-32, 34-55.

67. A method of darkening skin comprising the step of applying a composition according to any one of statements 1-24 onto a desired skin surface, or applying one of more compounds according to any one of statements 25-26, or one or more compounds of formula (I), (II), (III) or (IV), (IIb), (IIa), (IIIa), (Ie), (If), (Ig), (Ic), or (Id), or a racemate, a stereoisomer, a tautomer, a salt, and/or a solvate thereof, including mixtures thereof, according to any one of statements 27-32, 34-55 onto a desired skin surface, preferably wherein said method is non-therapeutic.

68. A compound of formula (I), (II), (III) or (IV) or a racemate, a stereoisomer, a tautomer, a salt, and/or a solvate thereof, including mixtures thereof, or a compound according to any one of statements 25-26, for use in increasing the amount of melanin in melanocytes, for increasing melanin synthesis, for improving melanin transport, and/or for improving the distribution of melanin in suprabasal layers,

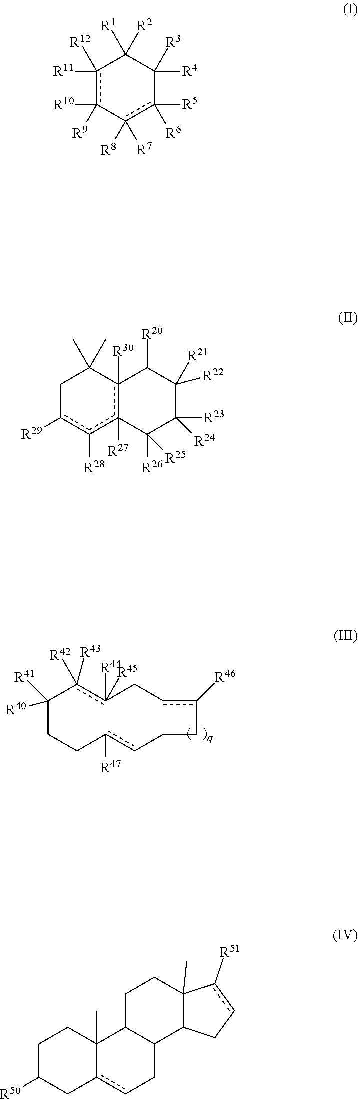

wherein the dash bond [symbol] represents an optional double bond; and wherein
R¹, R² are each independently selected from hydrogen or C₁₋₆alkyl;
R³, R⁴ are each independently selected from hydrogen or C₁₋₆alkyl;
or R¹ with R³ together with the carbon atoms to which they are attached form a saturated C3 to C5-membered ring, wherein said ring is optionally substituted with one or more C₁₋₆alkyl substituents;
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —X—[CR¹³R¹⁴]ₙ—O—[CR¹⁷R¹⁸]ₘ—OH, —([CR¹⁷R¹⁸]ₘ—O)_{y}—C₁₋₆alkyl, —[CR¹³R¹⁴]ₙ—CO—R¹⁹;
m is an integer selected from 1, 2, 3, 4 or 5, n is an integer selected from 0, 1, 2, 3 or 4; y is an integer selected from 1, 2 or 3;
X is a single bond or O,
R¹³, R¹⁴, R¹⁶, R¹⁷, R¹⁸, R¹⁹ are each independently selected from hydrogen or C₁₋₆alkyl;
R¹⁵ is OH, or R¹⁵ together with R¹⁶ form an oxo group;
or R³ and R⁵ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2;
or R⁵ and R⁶ form together ═[CR¹⁴]—[CR¹⁷R¹⁸]ₘ—OH; or R⁵ and R⁶ together with the carbon atom to which they are attached form a 5- or 6-membered heterocyclyl moiety, wherein said heterocyclyl is optionally substituted with one or more C₁₋₆alkyl substituents;
or R⁵ and R⁸ together with the carbon atoms to which they are attached form a saturated or unsaturated C5 or C6-membered ring, wherein said ring is optionally substituted with one or more substituents each independently selected from oxo, C₁₋₆alkyl, —O—C₁₋₆alkyl, —CO—C₁₋₆alkyl, —[CR¹⁷R¹⁸]ₘ—O—CO—C₁₋₆alkyl, —([CR¹⁷R¹⁸]ₘ—O)_{y}—C₁₋₆alkyl, —[CR¹⁷R¹⁸]ₘ—COH, or —[CR¹⁷R¹⁸]ₘ—OH; or two substituents on said ring form together with the carbon atoms to which they are attached a 3- to 6-membered heterocyclyl moiety, wherein said heterocyclyl moiety is optionally substituted with one or more substituents each independently selected from C₁₋₆ alkyl or oxo, or two substituents on said heterocyclyl form together with the atoms to which they are attached a 3- to 6-membered heterocyclyl moiety;
or R⁶ and R⁹ together with the carbon atoms to which they are attached form a C3-membered ring;
R⁶ if present is hydrogen or C₁₋₆alkyl;
R⁷ if present is hydrogen or C₁₋₆alkyl;
R⁸ is hydrogen or C₁₋₆alkyl;
or R¹ with R⁸ together with the carbon atoms to which they are attached form a saturated C5-membered ring, wherein said ring is optionally substituted with one or more C₁₋₆alkyl substituents;
R⁹, R¹⁰ are each independently selected from hydrogen or C₁₋₆alkyl;
or R¹ with R⁹ together with the carbon atoms to which they are attached form a saturated C5-membered ring, wherein said ring is optionally substituted with one or more C₁₋₆alkyl substituents;
or R³ with R⁹ together with the carbon atoms to which they are attached form a saturated C5-membered ring, wherein said ring is optionally substituted with one or more C₁₋₆alkyl substituents;
R¹¹, R¹² are each independently selected from hydrogen or C₁₋₆alkyl;
or R⁹ and R¹¹ together with the carbon atoms to which they are attached form a saturated or unsaturated C5 or C6-membered ring, wherein said ring is optionally substituted with one or more C₁₋₆alkyl substituents;
R²⁰ is hydrogen or C₁₋₆alkyl;
R²¹ is hydrogen or C₁₋₆alkyl;
R²² is selected from the group comprising hydrogen, C₁₋₆alkyl, and —CO—R¹⁹;
R²³ is hydrogen or C₁₋₆alkyl;
R²⁴ is hydrogen or C₁₋₆alkyl;
or R²³ and R²⁴ together with the carbon atom to which they are attached form a 3- to 6-membered heterocyclyl moiety; wherein said heterocyclyl is optionally substituted with one or more substituents each independently selected from C₁₋₆alkyl;
R²⁵ is hydrogen or C₁₋₆alkyl;
or R²⁴ and R²⁵ form together with the carbon atom to which they are attached a 5 or 6-membered heterocyclyl moiety, wherein said heterocyclyl is optionally substituted with one or more substituents each independently selected from C₁₋₆alkyl or oxo, or wherein one of said substituent and R²³ form together with the atoms to which they are attached a 3- to 6-membered heterocyclyl moiety;
R²⁶ is hydrogen or C₁₋₆alkyl;
R²⁷ if present is hydrogen or C₁₋₆alkyl;
or R²⁰ and R²⁷ together with the carbon atoms to which they are attached form a C3-membered ring;
R²⁸ is selected from the group comprising hydrogen, oxo, C₁₋₆alkyl, —O—C₁₋₆alkyl, —[CR¹⁷R¹⁸]ₘ—O—CO—C₁₋₆alkyl, —[CR¹⁷R¹⁸]ₘ—O—C₁₋₆alkyl, —[CR¹⁷R¹⁸]ₘ—COH, and —[CR¹⁷R¹⁸]ₘ—OH;
R²⁹ is hydrogen or C₁₋₆alkyl;
R³⁰ if present is hydrogen or C₁₋₆alkyl;
or R³⁰ with R²³ together with the carbon atoms to which they are attached form a saturated C3 to C5-membered ring, wherein said ring is optionally substituted with one or more C₁₋₆alkyl substituents;
or R²⁹ and R²⁸ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents;
q is an integer selected from 0, 1, 2, 3, preferably q is 1;
R⁴⁰ is selected from the group comprising —[CR¹⁷R¹⁸]ₘ—OH, —CO—C₁₋₆alkyl, —O—([CR¹⁷R¹⁸]ₘ—O—)ₜ—C₁₋₆alkyl, t is an integer selected from 0, 1, 2, or 3;
R⁴¹, R⁴², R⁴³, R⁴⁴, R⁴⁵, R⁴⁶, R⁴⁷ are each independently selected from hydrogen or C₁₋₆alkyl;
or R⁴⁰ and R⁴² together with the carbon atom to which they are attached form a 3- to 6-membered heterocyclyl moiety;
R⁵⁰ is selected from OH or oxo; and
R⁵¹ is hydrogen or C₁₋₁₀ alkyl.

69. A self-tanning composition prepared using one or more compounds as defined in any one of statements 25-26, or one or more compounds of formula (I), (II), (III) or (IV), (IIb), (IIa), (IIIa), (Ie), (If), (Ig), (Ic), or (Id), or a racemate, a stereoisomer, a tautomer, a salt, and/or a solvate thereof, including mixtures thereof, according to any one of statements 27-32, 34-55 as self-tanning substance.

The use as defined herein or the composition as defined herein can comprise at least one compound of formula (I), (Ia), (Ib), (Ic), (Id), (Ie), (If), or (Ig), or a racemate, a stereoisomer, a tautomer, a salt, a solvate and/or a mixture thereof,

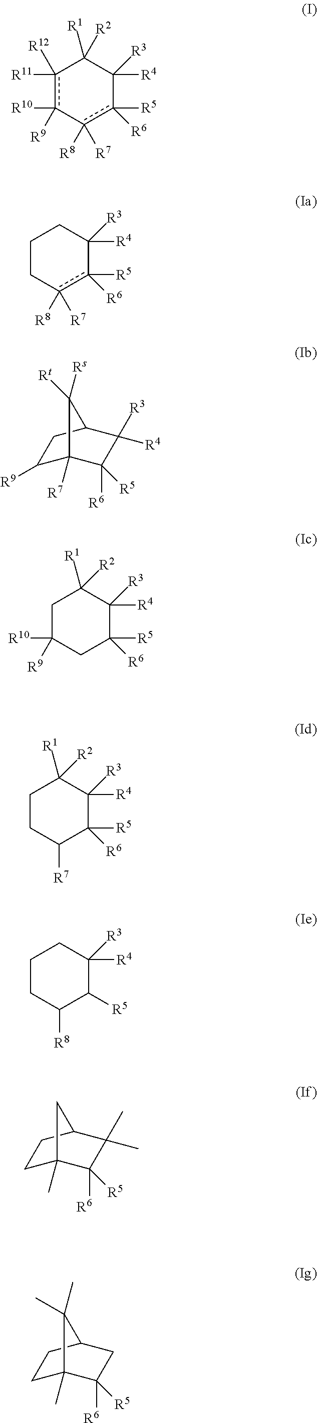

wherein the dash bond [symbol] represents an optional double bond; and wherein
R¹, R² are each independently selected from hydrogen or C₁₋₆alkyl; preferably R¹, R² are each independently selected from hydrogen or C₁₋₄alkyl; preferably R¹, R² are each independently selected from hydrogen or C₁₋₂alkyl; preferably R¹, R² are each independently selected from hydrogen or methyl;
R³, R⁴ are each independently selected from hydrogen or C₁₋₆alkyl; preferably R³, R⁴ are each independently selected from hydrogen or C₁₋₄alkyl; preferably R³, R⁴ are each independently selected from hydrogen or C₁₋₂alkyl; preferably R³, R⁴ are each independently selected from hydrogen or methyl;
or R¹ with R³ together with the carbon atoms to which they are attached form a saturated C3 to C5-membered ring, wherein said ring is optionally substituted with one or more C₁₋₆alkyl substituents; preferably R¹ with R³ together with the carbon atoms to which they are attached form a C3 to C4-membered ring, wherein said ring is optionally substituted with one or more C₁₋₄ alkyl substituents; preferably R¹ with R³ together with the carbon atoms to which they are attached form a C3-membered ring i.e. a cyclopropyl, wherein said cyclopropyl is optionally substituted with one or more C₁₋₂alkyl substituents; preferably wherein said cyclopropyl is optionally substituted with one or more methyl substituents;
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —X—[CR¹³R¹⁴]ₙ—O—[CR¹⁷R¹⁸]ₘ—OH, —([CR¹⁷R¹⁸]ₘ—O)_{y}—C1.6alkyl, —[CR¹³R¹⁴]ₙ—CO—R¹⁹; preferably R⁵ is selected from the group comprising —X—[CHR¹⁴]ₙ—C(═O)—[CHR¹⁸]ₘ—R¹⁹, —X—[CHR¹⁴]ₙ—CH(OH)—[CHR¹⁸]ₘ—R¹⁹, —X—[CHR¹⁴]ₙ—[CHR¹⁸]ₘ—OH, —X—[CHR¹⁴]ₙ—O—[CHR¹⁸]ₘ—OH, —([CHR¹⁸]ₘ—O)_{y}—C₁₋₆alkyl, —[CHR¹⁴]ₙ—CO—R¹⁹; preferably R⁵ is selected from the group comprising —O—[CHR¹⁴]ₙ—C(═O)—[CHR¹⁸]ₘ—R¹⁹, —[CHR¹⁴]ₙ—C(═O)—[CHR¹⁸]ₘ—R¹⁹, —O—[CHR¹⁴]ₙ—CH(OH)—[CHR¹⁸]ₘ—R¹⁹, —[CHR¹⁴]ₙ—CH(OH)—[CHR¹⁸]ₘ—R¹⁹, —O—[CHR¹⁴]ₙ—[CHR¹⁸]ₘ—OH, —[CHR¹⁴]ₙ—[CHR¹⁸]ₘ—OH, —CO—R¹⁹;
m is an integer selected from 1, 2, 3, 4 or 5, n is an integer selected from 0, 1, 2, 3 or 4; y is an integer selected from 1, 2 or 3; preferably m is selected from 1, 2 or 3, preferably m is 1 or 2, preferably n is selected from 0, 1, 2, or 3; preferably n is selected from 0, 1, or 2; preferably y is 1, or 2;
X is a single bond or O,
R¹³, R¹⁴, R¹⁶, R¹⁷, R¹⁸, R¹⁹ are each independently selected from hydrogen or C₁₋₆alkyl; preferably R¹³, R¹⁴, R¹⁶, R¹⁷, R¹⁸, R¹⁹ are each independently selected from hydrogen or C₁₋₄alkyl; preferably R¹³, R¹⁴, R¹⁶, R¹⁷, R¹⁸, R¹⁹ are each independently selected from hydrogen or C₁₋₂alkyl;
R¹⁵ is OH, or R¹⁵ together with R¹⁶ form an oxo group;
or R³ and R⁵ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2, R¹⁷, R¹⁸, are each independently selected from hydrogen or C₁₋₆alkyl; preferably R³ and R⁵ together are of formula —O—[CR¹⁷R¹⁸]—O—, and form together with the carbon atoms to which they are attached a 5-membered heterocyclyl moiety, wherein said heterocyclyl is optionally substituted with one or more C₁₋₆alkyl substituents; preferably R³ and R⁵ together are of formula —O—[CR¹⁷R¹⁸]—O—, and form together with the carbon atoms to which they are attached a dioxolanyl moiety, R¹⁷, R¹⁸, are each independently selected from hydrogen or C₁₋₄alkyl, wherein said dioxolanyl moiety is optionally substituted with one or two C₁₋₄alkyl substituents, preferably one or two C₁₋₂alkyl substituents, preferably one or two methyl;
or R⁵ and R⁶ form together ═[CR¹⁴]—[CR¹⁷R¹⁸]ₘ—OH; or R⁵ and R⁶ together with the carbon atom to which they are attached form a 5- or 6-membered heterocyclyl moiety, wherein said heterocyclyl is optionally substituted with one or more C₁₋₆alkyl substituents; preferably R⁵ and R⁶ form together ═[CH]—[CHR¹⁸]ₘ—OH; and m is 1 or 2; or R⁵ and R⁶ together with the carbon atom to which they are attached form a 6-membered heterocyclyl moiety, wherein said heterocyclyl is optionally substituted with one or more C₁₋₆alkyl substituents; preferably R⁵ and R⁶ form together ═[CH]—[CH₂]ₘ—OH; and m is 1 or 2; or R⁵ and R⁶ together with the carbon atom to which they are attached form a 6-membered dioxanyl moiety, for example 1,3-dioxanyl, wherein said dioxanyl is optionally substituted with one or more C₁₋₆alkyl substituents, for example one or more C₁₋₄alkyl substituents, one or more C₁₋₃alkyl substituents;
or R⁵ and R⁸ together with the carbon atoms to which they are attached form a saturated or unsaturated C5 or C6-membered ring, wherein said ring is optionally substituted with one or more substituents each independently selected from oxo, C₁₋₆alkyl, —O—C₁₋₆alkyl, —CO—C₁₋₆alkyl, —[CR¹⁷R¹⁸]ₘ—O—CO—C₁₋₆alkyl, —([CR¹⁷R¹⁸]ₘ—O)_{y}—C₁₋₆alkyl, —[CR¹⁷R¹⁸]ₘ—COH, or —[CR¹⁷R¹⁸]ₘ—OH; or two substituents on said ring form together with the carbon atoms to which they are attached a 3- to 6-membered heterocyclyl moiety, wherein said heterocyclyl moiety is optionally substituted with one or more substituents each independently selected from C₁₋₆alkyl, or two substituents on said heterocyclyl form together with the atoms to which they are attached a 3- to 6-membered heterocyclyl moiety; preferably R⁵ and R⁸ together with the carbon atoms to which they are attached form a saturated or unsaturated C6-membered ring, wherein said ring is optionally substituted with one or more substituents each independently selected from oxo, C₁₋₆alkyl, —O—C₁₋₆ alkyl, —CO—C₁₋₆alkyl, —[CR¹⁷R¹⁸]ₘ—O—CO—C₁₋₆alkyl, —[CR¹⁷R¹⁸]ₘ—O—C₁₋₆alkyl, —[CR¹⁷R¹⁸]ₘ—COH, or —[CR¹⁷R¹⁸]ₘ—OH; or two substituents on said ring form together with the carbon atoms to which they are attached a 3- to 6-membered heterocyclyl moiety such as a oxiranyl, tetrahydrofuranyl, dioxolanyl, tetrahydropyranyl, wherein said heterocyclyl moiety is optionally substituted with one or more substituents each independently selected from C₁₋₆alkyl, for example C₁₋₄alkyl, for example C₁₋₂alkyl, or two substituents on said heterocyclyl form together with the atoms to which they are attached a 5- to 6-membered heterocyclyl moiety such as a dioxanyl;
or R⁶ and R⁹ together with the carbon atoms to which they are attached form a C3-membered ring;
R⁶ if present is hydrogen or C₁₋₆alkyl; preferably R⁶ if present is hydrogen or C₁₋₄alkyl; preferably R⁶ if present is hydrogen or C₁₋₂alkyl; preferably R⁶ if present is hydrogen or methyl;
R⁷ if present is hydrogen or C₁₋₆alkyl; preferably R⁷ if present is hydrogen or C₁₋₄alkyl; preferably R⁷ if present is hydrogen or C₁₋₂alkyl; preferably R⁷ if present is hydrogen or methyl;
R⁸ is hydrogen or C₁₋₆alkyl; preferably R^{s} is hydrogen or C₁₋₅alkyl; preferably R⁸ is hydrogen or C₁₋₄ alkyl;
or R¹ with R⁸ together with the carbon atoms to which they are attached form a saturated C5-membered ring, wherein said ring is optionally substituted with one or more C₁₋₆alkyl substituents; preferably wherein said ring is optionally substituted with one or more C₁₋₄alkyl substituents; preferably wherein said ring is optionally substituted with one or more C₁₋₂alkyl substituents;
R⁹, R¹⁰ are each independently selected from hydrogen or C₁₋₆alkyl; preferably R⁹, R¹⁰ are each independently selected from hydrogen or C₁₋₄alkyl; R⁹, R¹⁰ are each independently selected from hydrogen or C₁₋₂alkyl;
or R¹ with R⁹ together with the carbon atoms to which they are attached form a saturated C5-membered ring, wherein said ring is optionally substituted with one or more C₁₋₆alkyl substituents; preferably wherein said ring is optionally substituted with one or more C₁₋₄alkyl substituents; preferably wherein said ring is optionally substituted with one or more C₁₋₂alkyl substituents;
or R³ with R⁹ together with the carbon atoms to which they are attached form a saturated C5-membered ring, wherein said ring is optionally substituted with one or more C₁₋₆alkyl substituents; preferably wherein said ring is optionally substituted with one or more C₁₋₄alkyl substituents; preferably wherein said ring is optionally substituted with one or more C₁₋₂alkyl substituents;
R¹¹, R¹² are each independently selected from hydrogen or C₁₋₆alkyl; preferably R¹¹, R¹² are each independently selected from hydrogen or C₁₋₄alkyl; preferably R¹¹, R¹² are each independently selected from hydrogen or C₁₋₂alkyl; preferably R¹¹, R¹² are each independently selected from hydrogen or methyl;
or R⁹ and R¹¹ together with the carbon atoms to which they are attached form a saturated or unsaturated C5 or C6-membered ring, wherein said ring is optionally substituted with one or more C₁₋₆alkyl substituents; preferably wherein said ring is optionally substituted with one or more C₁₋₄alkyl substituents; preferably wherein said ring is optionally substituted with one or more C₁₋₂alkyl substituents.

The use as defined herein or the composition as defined herein can comprise at least one compound of formula (II), (IIa), or (IIb), or a racemate, a stereoisomer, a tautomer, a salt, a solvate, and/or a mixture thereof,

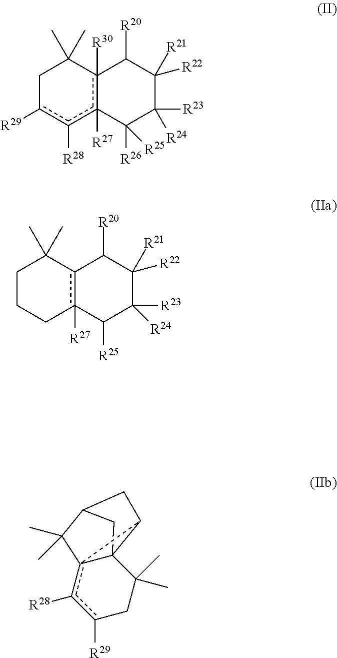

wherein the dash bond [symbol] represents an optional double bond; and wherein
R²⁰ is hydrogen or C₁₋₆alkyl; preferably R²⁰ is hydrogen or C₁₋₄alkyl; preferably R²⁰ is hydrogen or C₁₋₂alkyl; preferably R²⁰ is hydrogen or methyl;
R²¹ is hydrogen or C₁₋₆alkyl; preferably R²¹ is hydrogen or C₁₋₄alkyl; preferably R²¹ is hydrogen or C₁₋₂alkyl; preferably R²¹ is hydrogen or methyl;
R²² is selected from the group comprising hydrogen, C₁₋₆alkyl, and —CO—R¹⁹; with R¹⁹ being hydrogen or C₁₋₆alkyl; preferably R²² is selected from the group comprising hydrogen, C₁₋₄alkyl, and —CO—R¹⁹; with R¹⁹ being hydrogen or C₁₋₄alkyl; preferably R²² is selected from the group comprising hydrogen, C₁₋₂alkyl, and —CO—R¹⁹; with R¹⁹ being hydrogen or C₁₋₂alkyl; preferably R²² is selected from the group comprising hydrogen, methyl, and —CO-methyl;
R²³ is hydrogen or C₁₋₆alkyl; preferably R²³ is hydrogen or C₁₋₄alkyl; preferably R²³ is hydrogen, methyl or ethyl;
R²⁴ is hydrogen or C₁₋₆alkyl; preferably R²⁴ is hydrogen or C₁₋₄alkyl; preferably R²⁴ is hydrogen or C₁₋₂alkyl; preferably R²⁴ is hydrogen or methyl;
or R²³ and R²⁴ together with the carbon atom to which they are attached form a 3- to 6-membered heterocyclyl moiety; wherein said heterocyclyl is optionally substituted with one or more substituents each independently selected from C₁₋₆alkyl; preferably R²³ and R²⁴ together with the carbon atom to which they are attached form a 3- to 5-membered heterocyclyl moiety; wherein said heterocyclyl is optionally substituted with one or more C₁₋₄alkyl substituents; preferably R²³ and R²⁴ together with the carbon atom to which they are attached form a 3- to 4-membered heterocyclyl moiety; wherein said heterocyclyl is optionally substituted with one or more C₁₋₂alkyl substituents; preferably R²³ and R²⁴ together with the carbon atom to which they are attached form a 3-heterocyclyl moiety, preferably oxiranyl; wherein said heterocyclyl is optionally substituted with one or more C₁₋₂alkyl substituents, preferably methyl;
R²⁵ is hydrogen or C₁₋₆alkyl; preferably R²⁵ is hydrogen or C₁₋₄alkyl; preferably R²⁵ is hydrogen or C₁₋₂alkyl; preferably R²⁵ is hydrogen or methyl;
or R²⁴ and R²⁵ form together with the carbon atom to which they are attached a 5 or 6-membered heterocyclyl moiety, wherein said heterocyclyl is optionally substituted with one or more substituents each independently selected from C₁₋₆alkyl, or wherein one of said substituent and R²³ form together with the atoms to which they are attached a 3- to 6-membered heterocyclyl moiety; preferably R²⁴ and R²⁵ form together with the carbon atom to which they are attached a 5 or 6-membered heterocyclyl moiety, wherein said heterocyclyl is optionally substituted with one or more substituents each independently selected from C₁₋₄alkyl, or wherein one of said substituent and R²³ form together with the atoms to which they are attached a 4- to 6-membered heterocyclyl moiety; preferably R²⁴ and R²⁵ form together with the carbon atom to which they are attached a 5 or 6-membered heterocyclyl moiety for example a tetrahydropyranyl or a tetrahydrofuranyl, wherein said heterocyclyl is optionally substituted with one or more substituents each independently selected from C₁₋₂alkyl, or wherein one of said substituent and R²³ form together with the atoms to which they are attached a 5- to 6-membered heterocyclyl moiety such as a dioxolanyl;
R²⁶ is hydrogen or C₁₋₆alkyl; preferably R²⁶ is hydrogen or C₁₋₄alkyl; preferably R²⁶ is hydrogen or C₁₋₂alkyl; preferably R²⁶ is hydrogen or methyl;
R²⁷ if present is hydrogen or C₁₋₆alkyl; preferably R²⁷ is hydrogen or C₁₋₄alkyl; preferably R²⁷ is hydrogen or C₁₋₂alkyl; preferably R²⁷ is hydrogen or methyl;
or R²⁰ and R²⁷ together with the carbon atoms to which they are attached form a C3-membered ring (cyclopropyl);
R²⁸ is selected from the group comprising hydrogen, oxo, C₁₋₆alkyl, —O—C₁₋₆alkyl, —[CR¹⁷R¹⁸]ₘ—O—CO—C₁₋₆alkyl, —[CR¹⁷R¹⁸]ₘ—O—C₁₋₆alkyl, —[CR¹⁷R¹⁸]ₘ—COH, and —[CR¹⁷R¹⁸]ₘ—OH; m is 1, 2, 3 or 4, and R¹⁷ and R¹⁸ are each independently selected from hydrogen or C₁₋₆alkyl; preferably R²⁸ is selected from the group comprising hydrogen, oxo, C₁₋₄alkyl, —O—C₁₋₄alkyl, —[CHR¹⁸]ₘ—O—CO—C₁₋₄ alkyl, —[CHR¹⁸]ₘ—O—C₁₋₄alkyl, —[CHR¹⁸]ₘ—COH, and —[CHR¹⁸]ₘ—OH, m is 1, 2, or 3, and R¹⁸ is hydrogen or C₁₋₄alkyl; preferably R²⁸ is selected from the group comprising hydrogen, oxo, C₁₋₃alkyl, —O—C₁₋₃alkyl, —[CHR¹⁸]ₘ—O—CO—C₁₋₃alkyl, —[CHR¹⁸]ₘ—O—C₁₋₃alkyl, —[CHR¹⁸]ₘ—COH, and —[CHR¹⁸]ₘ—OH, m is 1, 2, or 3, and R¹⁸ is hydrogen or C₁₋₃alkyl;
R²⁹ is hydrogen or C₁₋₆alkyl; preferably R²⁹ is hydrogen or C₁₋₄alkyl; preferably R²⁹ is hydrogen or C₁₋₂alkyl; preferably R²⁹ is hydrogen or methyl;
or R²⁹ and R²⁸ together are of formula —O—[CR¹⁷R¹⁸]ₚ—O—, p is an integer selected from 1 or 2; and form together with the carbon atoms to which they are attached a 5- or 6-membered heterocyclyl moiety, wherein said heterocyclyl is optionally substituted with one or more C₁₋₆alkyl substituents, and R¹⁷ and R¹⁸ are each independently selected from hydrogen or C₁₋₆alkyl; preferably R²⁹ and R²⁸ together are of formula —O—[CR¹⁷R¹⁸]—O—; and form together with the carbon atoms to which they are attached a dioxolanyl, wherein said dioxolanyl is optionally substituted with one or more C₁₋₄alkyl substituents, and R¹⁷ and R¹⁸ are each independently selected from hydrogen or C₁₋₄alkyl; preferably R²⁹ and R²⁸ together are of formula —O—[CR¹⁷R¹⁸]—O—; and form together with the carbon atoms to which they are attached a dioxolanyl, wherein said dioxolanyl is optionally substituted with one or more C₁₋₂alkyl substituents, for example C₁₋₂alkyl substituents; R¹⁷ and R¹⁸ are each independently selected from hydrogen or C₁₋₂alkyl;
R³⁰ if present is hydrogen or C₁₋₆alkyl; preferably R³⁰ is hydrogen or C₁₋₄alkyl; preferably R³⁰ is hydrogen or C₁₋₂alkyl; preferably R³⁰ is hydrogen or methyl;
or R³⁰ with R²³ together with the carbon atoms to which they are attached form a saturated C3 to C5-membered ring, wherein said ring is optionally substituted with one or more C₁₋₆alkyl substituents; preferably R³⁰ with R²³ together with the carbon atoms to which they are attached form a saturated C4- to C5-membered ring, wherein said ring is optionally substituted with one or more C₁₋₄alkyl substituents; preferably R³⁰ with R²³ together with the carbon atoms to which they are attached form a saturated C5-membered ring (cyclopentyl), wherein said ring is optionally substituted with one or more C₁₋₂alkyl substituents.

The use as defined herein or the composition as defined herein can comprise at least one compound of formula (III), or (IIIa), or a racemate, a stereoisomer, a tautomer, a salt, a solvate and/or a mixture thereof,

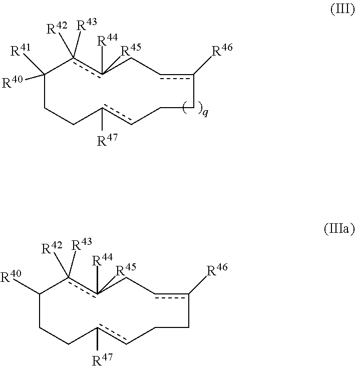

wherein the dash bond [symbol] represents an optional double bond; and wherein
q is an integer selected from 0, 1, 2, 3, preferably q is 0, 1 or 2, preferably q is 1 or 2; preferably q is 1;
R⁴⁰ is selected from the group comprising —[CR¹⁷R¹⁸]ₘ—OH, —CO—C₁₋₆alkyl, —O—([CR¹⁷R¹⁸]ₘ—O—)ₜ—C₁₋₆alkyl, t is an integer selected from 0, 1, 2, or 3; m is an integer selected from 1, 2, 3 or 4, and R¹⁷ and R¹⁸ are each independently selected from hydrogen or C₁₋₆alkyl; preferably R⁴⁰ is selected from the group comprising —[CHR¹⁸]ₘ—OH, —CO—C₁₋₆alkyl, —O—([CHR¹⁸]ₘ—O—)ₜ—C₁₋₆alkyl, t is an integer selected from 0, 1, or 2; m is an integer selected from 1, 2, or 3, and R¹⁸ is hydrogen or C₁₋₆alkyl; preferably R⁴⁰ is selected from the group comprising —[CHR¹⁸]ₘ—OH, —CO—C₁₋₄alkyl, —O—([CHR¹⁸]ₘ—O—)ₜ—C₁₋₄alkyl, t is an integer selected from 0, or 1; m is an integer selected from 1, or 2, and R¹⁸ is hydrogen or C₁₋₄alkyl; preferably R⁴⁰ is selected from the group comprising —[CHR¹⁸]ₘ—OH, —CO—C₁₋₂alkyl, —O—[CHR¹⁸]ₘ—O—C₁₋₂alkyl, m is an integer selected from 1, or 2, and R¹⁸ is hydrogen or C₁₋₂alkyl;
R⁴¹, R⁴², R⁴³, R⁴⁴, R⁴⁵, R⁴⁶, R⁴⁷ are each independently selected from hydrogen or C₁₋₆alkyl; preferably R⁴¹, R⁴², R⁴³, R⁴⁴, R⁴⁵, R⁴⁶, R⁴⁷ are each independently selected from hydrogen or C₁₋₄alkyl; preferably R⁴¹, R⁴², R⁴³, R⁴⁴, R⁴⁵, R⁴⁶, R⁴⁷ are each independently selected from hydrogen or C₁₋₂alkyl; preferably R⁴¹, R⁴², R⁴³, R⁴⁴, R⁴⁵, R⁴⁶, R⁴⁷ are each independently selected from hydrogen or methyl;
or R⁴⁰ and R⁴² together with the carbon atom to which they are attached form a 3- to 6-membered heterocyclyl moiety; preferably or R⁴⁰ and R⁴² together with the carbon atom to which they are attached form a 3- to 5-membered heterocyclyl moiety; or R⁴⁰ and R⁴² together with the carbon atom to which they are attached form a 3- to 4-membered heterocyclyl moiety; or R⁴⁰ and R⁴² together with the carbon atom to which they are attached form a 3-membered heterocyclyl moiety, preferably a oxiranyl moiety.

The use as defined herein or the composition as defined herein can comprise at least one compound of formula (IV), or a racemate, a stereoisomer, a tautomer, a salt, a solvate and/or a mixture thereof,

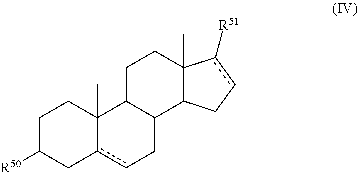

wherein the dash bond [symbol] represents an optional double bond; and wherein
R⁵⁰ is selected from OH or oxo; and R⁵¹ is hydrogen or C₁₋₁₀ alkyl, preferably R⁵¹ is hydrogen or C₁₋₄alkyl; preferably R⁵¹ is hydrogen or C₁₋₂alkyl; preferably R⁵¹ is hydrogen or methyl, preferably hydrogen.

The compounds for use in the invention can be added as a material to compositions, preparations for application to a subject's tissue, for example topical application, cosmetic products which can be used for increasing the pigmentation of a subject's tissue such increasing hair, skin, fur and/and feathers pigmentation by increasing the melanin production, coloring skin, or preventing, ameliorating, or treating symptoms such as skin sunburns, UVR's sensitivity; or can be used for producing these products. These compositions, preparations for example for topical application, cosmetic products, and the like, are also encompassed within the scope of the present invention.

The compounds, compositions, preparations, cosmetic products, can be produced or used for human or non-human animal. The compound for use herein can be the active ingredient for stimulating OR7C1 activity, increasing cAMP content increasing the melanin production, tanning skin, and/or preventing, ameliorating, and/or treating symptoms such as skin sunburns, UVR's sensitivity.

The compounds, compositions, preparations, cosmetic products can be administered by different administration forms. The administration may be oral or parenteral. Examples of the dosage form for oral administration include solid administration forms such as tablets, coated tablets, granules, powders, and capsules, and liquid administration forms such as elixirs, syrups and suspensions; examples of the dosage form for parenteral administration include injections, infusions, topicals, preparations for topical application, subcutaneous, transmucosal, transnasal, enteric, inhalation, suppositories, bolus, and patches. The composition can be preferably in the form of preparations for topical application to the skin.

The cosmetic composition comprises the compounds as the active ingredient. Examples of the cosmetic form include any form which can be used for a cosmetic product such as creams, emulsions, lotions, suspensions, gels, powders, packs, sheets, patches, sticks, and cake. The cosmetic compositions are preferably a skin tanning cosmetic product, and also preferably a cosmetic product for topical application to the skin. The cosmetic products are more preferably a cosmetic product for topical application for skin pigmentation.

The packaging could be a patch, bottle, tube, roll-ball applicator, propellant driven aerosol device, squeeze container or lidded jar.

The cosmetic compositions or cosmetic products may comprise the above defined compounds in combination with a pharmaceutically or cosmetically acceptable carrier, diluent and/or excipient.

The carrier can act as diluent or dispersant for the ingredients of the composition. The carrier may be aqueous-based, anhydrous or an emulsion, such as water-in-oil or oil-in-water emulsion.

In addition to water, organic solvents can be used as carrier or diluent in the compositions of the present invention. Non-limiting examples include alkanols like ethyl and isopropyl alcohol, and propane diol.

Other suitable organic solvents include ester oils like isopropyl myristate, cetyl myristate, 2-octyldodecyl myristate, avocado oil, almond oil, olive oil and neopentylglycol dicaprate. Typically, such ester oils assist in emulsifying the compositions.

Emollients may also be used, if desired, as a carrier. Alcohols like 1-hexadecanol (i.e., cetyl alcohol) are preferred. Other emollients include silicone oils and synthetic esters. Silicone oils suitable for use include cyclic or linear polydimethylsiloxanes containing from 3 to 9, preferably from 4 to 5 silicon atoms. Non-volatile silicone oils useful as emollients include polyalkyl siloxanes, polyalkylaryl siloxanes and polyether siloxane copolymers. Silicone elastomers may also be used.

The ester emollients that may optionally be used are:
(i) alkenyl or alkyl esters of fatty acids having 10 to 20 carbon atoms. Examples thereof include isoarachidyl neopentanoate, isononyl isonanonoate, oleyl myristate, oleyl stearate, and oleyl oleate;
(ii) ether-esters such as fatty acid esters of ethoxylated fatty alcohols;
(iii) polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters;
(iv) wax esters such as beeswax, spermaceti, stearyl stearate and arachidyl behenate; and,
(v) sterols esters, of which cholesterol fatty acid esters are examples.

Fatty acids having from 10 to 30 carbon atoms may also be included as carriers. Examples of such fatty acids include pelargonic, lauric, myristic, palmitic, stearic, isostearic, oleic, linoleic, arachidic, behenic or erucic acid, and mixtures thereof.

Humectants of the polyhydric alcohol type may also be employed in the compositions. The humectant often aids in increasing the effectiveness of the emollient, reduces scaling at the skin surface, stimulates removal of built-up scale and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol, and mixtures thereof. For best results, the humectant is preferably propylene glycol or sodium hyaluronate. Other humectants which may be used include hydroxyethyl urea.

Moisturization may be improved through use of petrolatum or paraffin. Thickeners may also be utilized as a portion of the carrier in the compositions. Typical thickeners include cross-linked acrylates (e.g., Carbopol® 982), hydrophobically-modified acrylates (e.g., Carbopol©1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums.

Surfactants may also be present. The surfactant is selected from the group consisting of anionic, nonionic, cationic and amphoteric actives. Particularly preferred nonionic surfactants are those with a C10-20 fatty alcohol or acid hydrophobe condensed with from 2 to 100 moles of ethylene oxide or propylene oxide per mole of hydrophobe; mono- and di-fatty acid esters of ethylene glycol; fatty acid monoglyceride; sorbitan, mono- and di-C8-C20 fatty acids; block copolymers (ethylene oxide/propylene oxide); and polyoxyethylene sorbitan, as well as combinations thereof. Alkyl polyglycosides and saccharide fatty amides (e.g., methyl gluconamides) are also suitable nonionic surfactants. Preferred anionic surfactants include soap, alkyl ether sulfate and sulfonates, alkyl sulfates and sulfonates, alkylbenzene sulfonates, alkyl and dialkyl sulfosuccinates, C8 to 20 acyl isethionates, acyl glutamates, C8 to 20 alkyl ether phosphates, and combinations thereof.

Various other ingredients may also be used in the compositions. Non-limiting examples of additional ingredients can be coating agent, binder, extender, disintegrator, lubricant, diluent, osmotic pressure regulator, pH regulator, dispersant, emulsifier, preservative, stabilizer, antioxidant, colorant, ultraviolet absorber, moisturizer, thickener, activity enhancer, anti-inflammatory agent, disinfecting agent, perfume, flavor, odor improver, and the like.

The compositions or cosmetic products may also comprise other active ingredients and pharmacological ingredients insofar as OR7C1 activating activity effects of the compounds are not affected.

The compositions or cosmetic products may also comprise other active ingredients and cosmetic ingredients such as a moisturizer, skin tanning agent, UV protector, cell activator, cleaner, keratolytic agent, and make-up components (e.g., a makeup base, foundation, face finishing powder, powder, cheek color, rouge, eye makeup, eyebrow pencil, mascara, etc.) insofar as OR7C1 activating activity effects of the compound are not affected.

The above compositions or cosmetic products can be produced by a routine method combining the compounds as necessary with the carrier, diluent, and/or excipient, and/or other active ingredients, cosmetic ingredients, or pharmacological ingredients. For example, the composition for topical application to the skin can be prepared using ingredients typically added to preparations for topical application, pharmaceutical products for topical application or cosmetic products for the skin such as oils or oily substances (e.g., fats or oils, waxes, higher fatty acids, essential oils, silicone oils, or the like), moisturizers (e.g., glycerol, sorbitol, gelatin, polyethylene glycol, or the like), powders (e.g., chalks, talcs, Fuller's earth, kaolin, starch, rubber, or the like), dye, emulsifier, solubilizer, cleaner, ultraviolet absorber, thickener, medicinal component, perfume, resin, antibacterial and antifungal agent, other plant extracts (e.g., crude drugs, Kampo products, herbs), alcohols, polyvalent alcohols, inorganic acids (e.g., bicarbonate, carbonate, sodium chloride, potassium chloride, sodium sulfate, or the like), organic acids (e.g., succinic acid, glutaric acid, fumaric acid, glutamic acid, malic acid, citric acid, ascorbic acid, or the like), vitamins (e.g., vitamin As, vitamin Es, vitamin Bs, vitamin C, folic acid, or the like), water-soluble polymers, anionic surfactants (e.g., alkylbenzene sulfonate, alkylsulfate, or the like), cationic surfactants (e.g., alkyl quaternary ammonium salt, alkyl dimethyl benzyl ammonium salt, or the like), nonionic surfactants (e.g., polyoxyethylene alkyl ether, polyoxyethylene alkyl phenyl ether, or the like), and amphoteric surfactants (e.g., imidazoline or carbobetaine containing an alkyl group, or the like).

In the case of compositions for topical application, the content of the compound for use in the invention is, on a dry weight basis, preferably from 0.00001 to 20% by weight, more preferably from 0.0001 to 10% by weight.

It will be appreciated by those of skill in the art that light or very fair skin and/or hair is the most difficult to pigment and may require higher doses than would be necessary for less fair skin and/or hair. For example, subjects that are a Fitzpatrick scale Type I or Type II may require higher doses to enhance pigmentation than subjects that are a Type III or above. The Fitzpatrick scale is shown in the following table.

Fitzpatrick Scale of Skin Color

| Fitzpatrick Type | Scores | Characteristics |
|---|---|---|
| I | 0-6 | Pale white; blond or red hair; blue eyes; freckles—Always |
| | | burns, never tans |
| II | 7-13 | White; fair; blond or red hair; blue, green, or hazel eyes— |
| | | Usually burns, tans minimally |
| III | 14-20 | Cream white; fair with any hair or eye color; quite common— |
| | | Sometimes mild burn, tans uniformly |
| IV | 21-27 | Moderate brown; typical Mediterranean skin tone—Rarely |
| | | burns, always tans well |
| V | 28-34 | Dark brown; Middle Eastern skin types—Very rarely burns, tans |
| | | very easily |
| VI | 35-36 | Deeply pigmented dark brown to black—Never burns, tans very |
| | | easily |

The present invention is particularly suitable for subjects that are a Fitzpatrick scale Type I, Type II, Type III, and Type IV.

The invention encompasses a method of darkening skin comprising the step of applying a composition or a compound as hereinbefore disclosed onto the desired skin surface.

The method is preferably non-therapeutic in nature.

The suitable skin surface includes facial skin, hands, arms, feet, legs, neck, chest, back including the lower back, and scalp. Of interest is facial skin including the forehead, perioral, chin, periorbital, nose, and/or cheeks. The composition may be applied and left on the desired surface for a sufficient time or may be applied repeatedly a sufficient number of times. In certain embodiments, the contact time is greater than 1 hour, greater than 2 hours, greater than 6 hours, greater than 8 hours, greater than 12 hours, or greater than 24 hours. The contact time is the time from application of the composition until the composition is removed. In certain embodiments, the composition may be removed by rinsing or washing the substrate. The composition may be removed by washing or rinsing the skin. The composition may be applied at least once daily. In other embodiments, the composition is applied at least twice daily. Multiple applications may occur over the course of at least about one week. Alternately, the application period may last more than about 3 weeks or more than about 9 weeks. The composition may be applied daily for prolonged period.

According to yet another aspect, the invention provides for use of the composition as hereinbefore disclosed for darkening skin. The use is preferably cosmetic.

The invention also provides for use of a compound of formula (I), (II), (III), or (IV) or a subgroup thereof, or a racemate, a stereoisomer, a tautomer, a salt, and/or a solvate thereof, including mixtures thereof, in the manufacture of a composition to provide skin darkening.

The present invention provides a method for increasing the melanin in cells. The method comprises a step of adding a compound of formula (I), (II), (III), or (IV) or a subgroup thereof, or a racemate, a stereoisomer, a tautomer, a salt, and/or a solvate thereof, including mixtures thereof, or a composition, as described herein to a tyrosinase-expressing cell in which the tyrosinase production needs to be stimulated.

The present invention provides a method for increasing the melanin production in cells. The method comprises a step of adding a compound of formula (I), (II), (III), or (IV) or a subgroup thereof, or a racemate, a stereoisomer, a tautomer, a salt, and/or a solvate thereof, including mixtures thereof, or a composition, as described herein to a melanin-producing cell in which the melanin production needs to be stimulated.

In the present invention, the “cell” in which the melanin production is to be activated is not particularly limited, insofar as it is a tyrosinase-expressing or melanin-producing cell that is native or modified by a genetic engineering technique. The cell is preferably pigment cells (melanocyte, retinal pigment epithelial cell, or the like), with melanocytes being more preferable. Alternatively, the “cell” may be a cell debris or cell fraction of the cells mentioned above, tissues containing the cells mentioned above or cultured product derived from the cells mentioned above. When the cell is a cell cultured product, the cell is preferably cultured in the presence of the above formulations of invention. The concentration of the compound to be added is, when the cell is a cell cultured product, as the final concentration in the cultured product range between 0.01 μM to 1 mM, preferably from 0.05 μM to 500 μM, most preferably from 0.1 μM to 150 μM.

In the present invention, the compound or composition, as described herein, can be administered or ingested in an effective amount to or by a subject for increasing the melanin synthesis, tanning skin, or preventing, ameliorating, or treating symptoms such as skin sunburns, UVR's sensitivity. The administration or ingestion may be carried out non-therapeutically for health promotion or aesthetic purpose. Examples of the subject in the administration or ingestion include animals which need to stimulate the melanin synthesis. Alternatively, examples of the subject in the administration or ingestion include animals who desire the melanin production stimulation or skin tanning, or animals who desire the prevention, amelioration, or treatment of the symptoms such as skin sunburns or UVR's sensitivity. The animal is preferably a human or non-human mammal, with a human being preferable.

The effective amount of administration or ingestion may be an amount which can stimulate the melanin production in cells of the subject. The preferable amount of administration or ingestion is variable depending on species, body weight, sex, age, conditions of the subject, or other factors. The dose, route, interval of administration or injection, and the amount of ingestion and interval, can be suitably determined by those skilled in the art. For example, when topically administered to the human skin, the amount of administration per adult (60 kg) is from 0.01 μM to 1 mM, preferably from 0.05 μM to 500 μM, most preferably from 0.1 μM to 150 μM.

The invention will be explained in detail with the help of the following non-limiting examples.

### EXAMPLES

Experimental Procedures:

Cell Culture and Cell Line Generation

HEK293T-RTP1A1/RTP2 cells were maintained in minimal essential medium (EMEM, Lonza) containing 10% fetal bovine serum (M10). These cells were generated by transfecting HEK293T (Thermofisher Scientific, cat ref: R70007) with an expression vector containing the sequences of the chaperone proteins RTP1A1 and RTP2 and a resistance gene to puromycin, using Lipofectamine 2000. The recombinant cell population, used in these experiments, was selected by adding 10 μg/ml of puromycin into the culture medium and subsequently subcloned (WO 2014/191047 A1).

Experiments on melanin production were performed using Normal Human Epidermal Melanocytes-NHEMs-MP—(moderately pigmented, LIFE TECHNOLOGIES; ref:C-102-5C (LOT #706296)).

This primary culture was cultivated in medium M254 supplemented (LIFE TECHNOLOGIES; ref:M-254-500) with HMGS (human melanocyte growth supplement; LIFE TECHNOLOGIES; ref: S-002-5) and gentamycin (LIFE TECHNOLOGIES; ref:15710-049) and maintained in a humidified incubator at 37° C. with a 5% CO2 atmosphere. For experiments (see below), cells were seeded at a density of 9×10⁴ cells/well (100 μL/well) and cultured at 37° C. under a 5% CO₂.

Dilution of OR7C1 Agonists

Potential OR7C1 agonists were diluted at a concentration of 1 mole/liter (M) into an organic solvent such as Dimethyl Sulfoxide (DMSO) or Ethanol to generate stock solutions.

For screening experiments, compound stock solutions were diluted in EMEM disposed in 96-well plates. Plates containing the tested compounds (1 compound/well) at a concentration of 2 mM, at a concentration of 632 μM and at a concentration of 200 μM were prepared.

For concentration-response analysis, serial dilutions of the tested molecules were prepared from stock solutions in EMEM plated into 96-well plates.

Luciferase Assay

For the dose-response analysis, a Luciferase-based gene reporter assay (Promega, Leiden, the Netherlands) was used. Briefly, cells were platted on poly-D-lysine-coated 96-well plates and transfected with a plasmid containing the CRE-luciferase and a plasmid containing the olfactory receptor. Sixteen hours after transfection, the culture medium was replaced by serum-free EMEM containing the tested compounds at a determined concentration. After four hours of incubation at 37° C., cells were lysed and processed for luminescence measurement. Luminescence emission was recorded. Results were expressed as luciferase activity (Relative Fluorescence Unit (RLU)) or as percentage of the response induced by 10 μM of the adenylate cyclase activator Forskolin. An empty plasmid is used as negative control.

Total RNA Extraction

Total RNAs were extracted using the Qiagen RNeasy kit (Qiagen; ref: 74100). Cells were rinsed with PBS and lysed in the adequate buffer. The extractions were performed according to the supplier's recommendations. The collected RNAs were stored at −80° C.

cDNA Synthesis

cDNA synthesis Reverse Transcription was performed with the SuperScript III (Invitrogen; ref:18080_051) from 1 μg of total RNA according to the manufacturer's instructions. The cDNAs were then stored at −20° C. until the PCR reaction.

Polymerase Chain reaction (PCR)

PCR was performed on 50 ng of cDNA obtained after Reverse Transcription synthesis (see above). The primers used for the reaction were, for couple 1, forward: CATTCTCTTTGGGCTGTTCC (SEQ ID 1); reverse: TTATGAAAGTCCTGCAGTGATG (SEQ ID 2). For couple 2, the primers used were forward: ATGGAAACAGGAAATCAAACAC (SEQ ID 3); reverse: TTATGAAAGTCCTGCAGTGATG (SEQ ID 4) and for couple 3, forward: CATTCTCTTTGGGCTGTTCC (SEQ ID 5); reverse: GAAGGAAATCACACCCAGGA (SEQ ID 6). The expected amplification products were 889 pair bases (pb), 962 pb and 559 pb respectively. The positive PCR control used is the cDNA sequence of OR7C1 present in an expression plasmid (pEFIBRHO). The PCR products were run in 1% agarose gel containing Ethidium bromide according to the manufacturer's instructions (Thermo fisher Scientific, ref:15585011).

Cells Treatment and Cytotoxicity Test (MTS)

NHEMs moderately pigmented (NHEMs-MP) were seeded in 24-wells plates at a density of 9×10⁴ cells/well, 72 h before the treatment with the different concentrations of 2 different compounds according to embodiments of the invention. After 2 days of treatment with these compounds, compounds were re-applied for 2 additional days according to the experimental scheme as shown in FIG. 1. Sodium dodecyl sulfate (SDS) at 0.05% was used as cytotoxicity positive control in order to validate the experiment. A sample treated with 0.1% ethanol (corresponding to the test items solvent) was tested as well. After 4 days of treatment, the MTS test for cytotoxicity was performed according to the supplier's recommendations (Promega; ref: G3581).

Evaluation of the Effect of Compounds According to Embodiments of the Invention on Pigmentation of NHEMs-MP in Culture

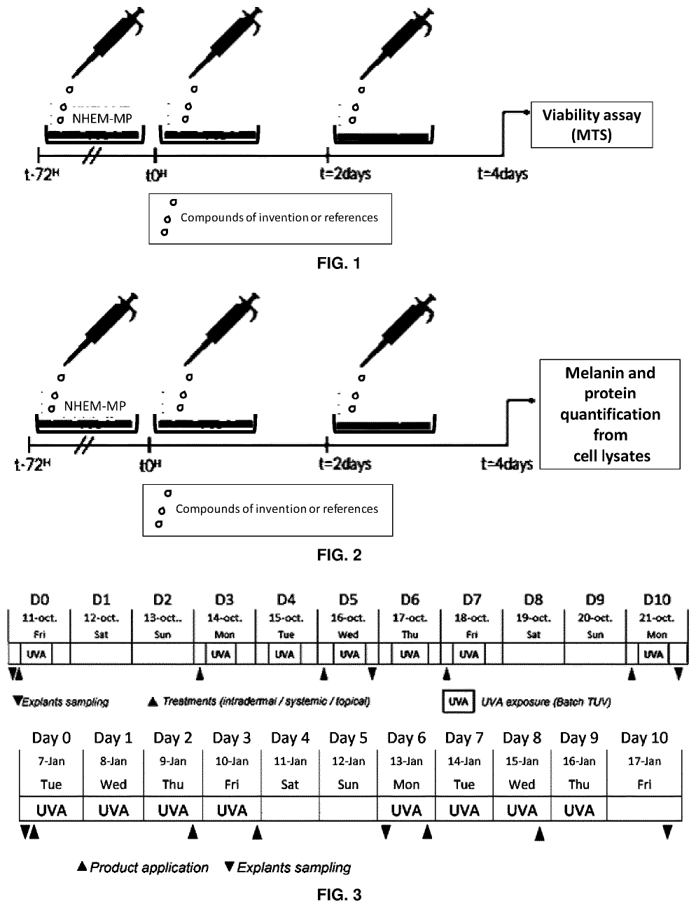

The NHEMs-MP were treated for 4 days, with a medium refresh after 2 days of treatment—Amberketal, Ambermax and with the reference molecule (L-DOPA)—when the culture media are collected and stored at −80° C. as shown in FIG. 2. At the end of the treatments, the culture media were collected a second time, pooled with the media collected at day 2 and centrifuged for 30 minutes (14000 rpm). The pellet obtained after centrifugation and NHEMs-MP treated cells were lysed in 150 μl of a NaOH 1M solution. 5 μl of each cell lysate were used for protein quantification using BCA protein assay. The remaining 145 μl of the lysates were heated at 80° C. during 1 h. The optical density was measured at 490 nm and the melanin content was determined by comparison with a standard curve of synthetic melanin and reported to the quantity of protein present in each cell well tested.

Evaluation of the Tanning Effect of Compounds of Invention

a. Explant Preparation:

Human skin explants of an average diameter of 12 mm (±1 mm) were prepared on an abdoplasty coming from a 47-year-old Caucasian woman (reference: P2237AB47—phototype II) or 61-years-old woman (reference P2196-AB61, phototype III). The explants were kept in survival in BEM culture medium (BIO-EC's Explants Medium) at 37° C. in a humid, 5%-CO₂ atmosphere.

b. Product Preparation:

The Ambermax was diluted in 0.1% ethanol at the following concentrations: 0.3 μM, 1 μM, 3 μM, 10 μM, 15 μM, 30 μM and 100 μM. The diluted solutions were stored at 4° C. during the study.

c. Study Schedule:

The explants were treated for 10 days with Ambermax (see products application below) or with no treatment. The Experimental scheme is shown in FIG. 3.

d. Products Application:

On day 0 (DO), day 3 (D3), day 5 (D5), day 7 (D7) and day 10 (D10), the tested products (compounds for use in the invention) were 1) applied topically using a small spatula based on 2 μl per explant (2 mg per cm²) at the indicated concentrations, 2) applied intradermatically by injections into the dermis using a syringe with 20 μL of product for each explant or 3) applied systemically by dilutions of the compound of invention in the culture media at the indicated concentration. The control explants (T) did not receive any treatment except the renewal of the culture medium. The culture medium was half renewed (1 ml/well) on day 3, day 5, day 7 and day 10.

or

On day 0 (D0), D2, D3, D6, and D8, the tested products (compounds for use in the invention) were applied topically using a small spatula on the basis of 2 μl per explant (2 mg per cm²). The control explants T did not receive any treatment except the renewal of the culture medium. The culture medium was half renewed (1 ml/well) on D2, D3, D6 and D8.

e. Chronic UVA-Irradiations:

On D0, D3, D4, D5, D6, D7 and D10, the explants of the batch TUV were put in 1 mL of HBSS (Hank's Balanced Saline Solution) and irradiated using a Vilber Lourmat RMX 3W UV simulator with a dose of 2.25 J/cm² of UVA (equivalent to 0.5 MED—Minimal Erythemal Dose).

or

On D0, D1, D2, D3, D6, D7, D8 and D9, the explants of the batch TUV were put in 1 mL of HBSS (Hank's Balanced Saline Solution) and irradiated using a Vilber Lourmat RMX 3W UV simulator with a dose of 2.25 J/cm² of UVA, equivalent to 0.5 MED (Minimal Erythemal Dose).

f. Sampling

On day 0 (D0), the 3 explants from the batch TO were collected and cut into three parts. One part was fixed in formalin buffered solution, the second part was frozen at −80° C. and the third part was placed in RNA later® solution.

On D5 and D10, the 3 explants of each batch were collected and processed in the same way than on day 0.

or

On D6 and D10, the 3 explants of each batch were collected and processed in the same way than on day 0.

According to the study plan, the days of treatments, irradiations and samplings were adjusted to fit the schedule of working days.

g. Histological Processing

After fixation for 24 hours in buffered formalin solution, the samples were dehydrated and impregnated in paraffin using a Leica PEARL dehydration automat. The samples were then paraffin-embedded using a Leica EG 1160 embedding station. 5-μm-thick sections of paraffin-embedded formol fixed sections were realized using a Leica RM 2125 Minot-type microtome, and the sections were then mounted on Superfrost® Plus glass slides. Microscopical observations were realized using a Leica DMLB, a BX43 or BX63 Olympus microscope. Pictures were digitized with an Olympus DP72 or DP74 camera and the Cell\hat{ }D or cellSens data storing software.

g.1. Melanin Visualization

Melanin was visualized after silver impregnation according to Masson's Fontana staining method on formalin-fixed paraffin-embedded (FFPE) skin sections. The staining was assessed by microscopical observation and image analysis of specific batches. Concerned batches: T0, (TUV, P1→P10) on D5 or D6 and D10.

g.2. Cellular Viability of the Explants

The cell viability of epidermal and papillary dermal structures was observed on formalin-fixed paraffin-embedded (FFPE) skin sections after Masson's trichrome staining, Goldner variant. The cell viability was assessed by microscopical observation. Concerned batches: T0, (TUV, P1→P10) on D5 or D6 and D10.

h. Image Analysis Method

The images analyses were performed on all the images of each batch, according to the following method using Cell\hat{ }D or cellSens software:

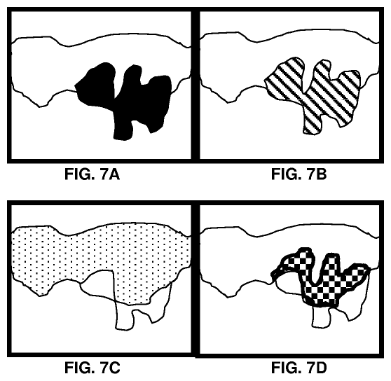

1. Image of the Immunostaining:
Schematic representation of an image to analyze Stained structure= Black as shown in Figure
7A. Stained structure = Black
2. Detection of the staining:
Selection of the pixels corresponding to the staining, by thresholding. Staining of interest=
striped mask as shown in Figure 7B
3. Determination of the region of interest (ROI):
Selection of the ROI by drawing. For example, here, the epidermis.
ROI/epidermis = spotted mask as shown in Figure 7C
4. Selection of the immunostaining in the region of interest:
Combination of the two masks: conserve common pixels to the spotted and striped mask.
Staining in the epidermis = checkered mask as shown in Figure 7D
5. Surface measurement of the surface of the spotted mask (epidermis), measurement of the
surface of the checkered mask (staining in the epidermis) and calculation of the percentage
of surface of the epidermis covered by the staining.
6. Results = > Exportation of the results (stained surface %) to Excel for analysis.

For each batch of explants, the percentage of the region of interest covered by the staining (stained surface percentage) is determined by image analysis according to the previously described process.

Stained surfaces comparisons: the stained surface percentage (Surf %) for each treatment is compared to the untreated condition=>P vs T. The same comparisons are made for the induced batches with or without treatment=>T vs TUV.

Statistical Analysis:

Student t-test gives the probability “p” for two treatments/groups to be significantly different. The difference between two treatments is significant if p<0.1 (#; limit of significancy), so a probability of 90% for two treatments/groups to be significantly different or p<0.05(*), so a probability of 95% for two treatments/groups to be significantly different or p<0.01(**), so a probability of 99% for two treatments/groups to be significantly different.

### Example 1: Organic Compounds Synthesis

### Synthesis of 1-((1,3,3-trimethylbicyclo[2.2.1]heptan-2-yl)oxy)pentan-2-ol (5)

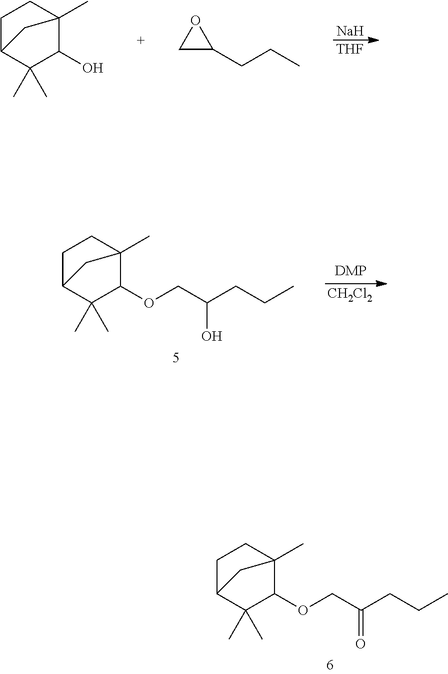

To a stirred solution of 1,3,3-trimethylbicyclo[2.2.1]heptan-2-ol (500 mg, 3.24 mmol) in THF (25 mL) were added sodium hydride (93 mg, 3.89 mmol) and 2-propyloxirane (279 mg, 3.24 mmol) at 0° C. under argon atmosphere. The reaction mixture was heated under microwave at 110° C. for 3 h. The reaction was monitored by TLC; after consumption of the starting material, the reaction mixture was diluted with water (20 mL) and extracted with ethyl acetate (2×20 mL). The combined organic extracts were dried over anhydrous Na₂SO₄, filtered and concentrated under reduced pressure. The crude material was purified by combi flash chromatography eluting with 3-4% ethyl acetate/Hexanes to afford title compound 5 (250 mg, 32%) as colorless oil.

¹H NMR (400 MHz, CDCl₃): δ 3.82-3.73 (m, 2H), 3.55-3.50 (m, 1H), 3.42-3.30 (m, 2H), 3.23-3.19 (m, 1H), 2.90-2.85 (m, 2H), 2.22-2.19 (m, 2H), 1.73-1.60 (m, 6H), 1.55-1.38 (m, 11H), 1.12-1.09 (m, 7H), 1.08-1.00 (m, 7H), 0.99-0.90 (m, 12H)

### Synthesis of 1-((1,3,3-trimethylbicyclo[2.2.1]heptan-2-yl)oxy) pentan-2-one (6)

To a stirred solution of 1-((1,3,3-trimethylbicyclo[2.2.1]heptan-2-yl)oxy) pentan-2-ol (100 mg, 0.41 mmol) in CH₂Cl₂ (10 mL) was added Dess martin periodinane (176 mg, 0.41 mmol) at 0° C. The reaction mixture was stirred at RT for 2 h. The reaction was monitored by TLC; after consumption of the starting material, the reaction mixture was diluted with saturated sodium bicarbonate solution (20 mL) and extracted with CH₂Cl₂ (3×20 mL). The combined organic extracts were dried over anhydrous Na₂SO₄, filtered and concentrated under reduced pressure. The crude material was purified by combi flash chromatography eluting with 5% ethyl acetate/Hexanes to afford title compound 6 (30 mg, 30%) as colorless oil.

¹H NMR (400 MHz, CDCl3): δ 4.11-3.97 (m, 1H), 3.97-3.87 (m, 1H), 2.92 (d, J=1.7 Hz, 1H), 2.54 (t, J=7.3 Hz, 2H), 1.85-1.67 (m, 2H), 1.65-1.59 (m, 2H), 1.55 (s, 2H), 1.48-1.36 (m, 1H), 1.14 (s, 3H), 1.12-1.08 (m, 1H), 1.05-1.02 (m, 4H), 0.97-0.90 (m, 6H)

### Synthesis of 1-((1,3,3-trimethylbicyclo[2.2.1]heptan-2-yl)oxy) butan-2-ol (7)

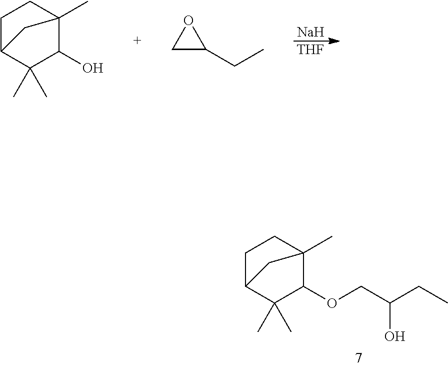

To a stirred solution of 1,3,3-trimethylbicyclo[2.2.1]heptan-2-ol (100 mg, 0.65 mmol) in THF (5 mL) were added sodium hydride (15 mg, 0.65 mmol) and 2-ethyloxirane (48 mg, 0.65 mmol) under argon atmosphere at RT. The reaction mixture was heated under microwave at 120° C. for 30 min. The reaction was monitored by TLC; after consumption of the starting material, the reaction mixture was diluted with water (20 mL) and extracted with ethyl acetate (2×20 mL). The combined organic extracts were dried over anhydrous Na₂SO₄, filtered and concentrated under reduced pressure. The crude material was purified by combi flash chromatography eluting with 3-4% ethyl acetate/Hexane to afford title compound 7 (50 mg, 34%) as colorless oil.

¹H NMR (400 MHz, CDCl3): δ 3.73-3.59 (m, 2H), 3.56-3.53 (m, 1H), 3.44-3.34 (m, 2H), 3.27-3.17 (m, 1H), 2.91-2.89 (m, 2H), 2.43-2.40 (m, 2H), 1.79-1.61 (m, 6.67H), 1.54-1.23 (m, 11.95H), 1.13-1.07 (m, 8.99H), 1.05-1.00 (m, 7.31H), 0.99-0.94 (m, 8.29H), 0.91-0.84 (m, 12H)

### Synthesis of 1-((2,2,6-trimethylcyclohexyl)oxy) pentan-2-ol (14)

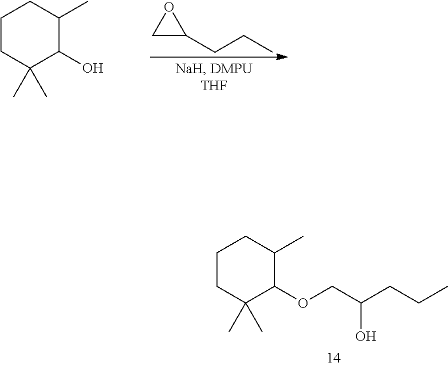

To a stirred solution of 2,2,6-Trimethylcyclohexan-1-ol (200 mg, 1.40 mmol) in THF (10 mL) were added sodium hydride (33 mg, 1.40 mmol) at 0° C. and stirred for 2 h. Then DMPU (23 mg, 1.40 mmol) and 2-propyloxirane (121 mg, 1.40 mmol) were added to the reaction mixture at 0° C. The reaction mixture was heated to 80° C. for 12 h. The reaction was monitored by TLC; after consumption of the starting material, the reaction mixture was diluted with water (20 mL) and extracted with ethyl acetate (2×20 mL). The combined organic extracts were dried over anhydrous Na₂SO₄, filtered and concentrated under reduced pressure. The crude material was purified by combi flash chromatography eluting with 3-4% Ethyl acetate/Hexane to afford title compound 14 (60 mg, 8%) as diastereomeric mixture as colorless liquid.

¹H NMR (400 MHz, CDCl₃): δ 3.84-3.73 (m, 1H), 3.61-3.50 (m, 1H), 3.49-3.37 (m, 1H), 2.50-2.41 (m, 2H), 1.72-1.58 (m, 2H), 1.54-1.31 (m, 7H), 1.01-0.86 (m, 14H)

### Synthesis of 1-((2,2,6-trimethylcyclohexyl)oxy) butan-2-ol (15)

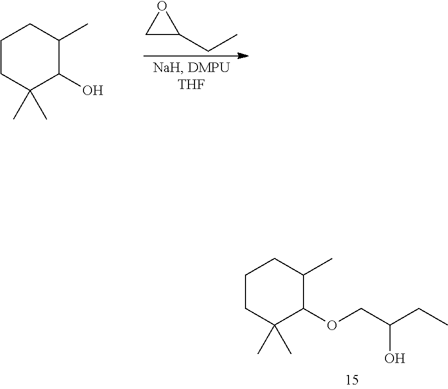

To a stirred solution of 2,2,6-Trimethylcyclohexan-1-ol (200 mg, 1.40 mmol) in THF (10 mL) were added sodium hydride (33 mg, 1.40 mmol) at 0° C. and stirred for 2 h. Then DMPU (23 mg, 1.40 mmol) and 2-ethyloxirane (101 mg, 1.40 mmol) were added to the reaction mixture at 0° C. The reaction mixture was heated to 80° C. for 12 h. The reaction was monitored by TLC; after consumption of the starting material, the reaction mixture was diluted with water (20 mL) and extracted with Ethyl acetate (2×20 mL). The combined organic extracts were dried over anhydrous Na₂SO₄, filtered and concentrated under reduced pressure. The crude material was purified by combi flash chromatography eluting with 3-4% Ethyl acetate/Hexane to afford title compound 15 (50 mg, 7%) as diastereomeric mixture as colorless oil.

¹H NMR (400 MHz, CDCl₃): δ 3.75-3.66 (m, 1H), 3.62-3.51 (m, 1H), 3.50-3.39 (m, 1H), 2.48 (d, J=10.1 Hz, 1H), 2.43 (dd, J=3.2, 1.0 Hz, 1H), 1.71-1.31 (m, 8H), 1.27-1.13 (m, 1H), 1.00-0.93 (m, 12H)

### Example 2: Dose-Response Analyses of Organic Compounds —OR Interactions

Most test compounds were purchased from the sources given below in Table 1. Compounds 5, 6, 7, 14, 17 were prepared as described in Example 1.

TABLE 1
Compound

| | | | | Provider or ref |
|---|---|---|---|---|
| Name | Structure | Number | CAS | for synthesis |
| Norlimbanol | 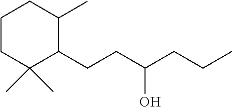 [SMILES: CCCC(O)CCC1C(C)CCCC1(C)C] | 1 | 70788-30-6 | Perfumer Supply house LLC |
| Ambercore | 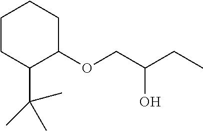 [SMILES: CCC(O)COC1CCCCC1C(C)(C)C] | 2 | 139504-68-0 | Pell Wall |
| Grisalva | 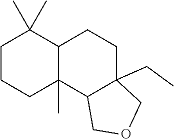 [SMILES: CCC12CCC3C(C)(C)CCCC3(C)C1COC2] | 3 | 68611-23-4 | Perfumers Apprentice |
| Georgywood | 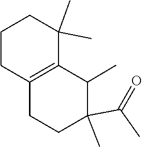 [SMILES: CC(=O)C1(C)CCC2=C(C1C)C(C)(C)CCC2] | 4 | 185429-83-8 | J. Am. Chem. Soc. 2006, 128, 4, 1346- 1352 |
| 1-((1,3,3- trimethylbicyclo[2.2.1] heptan-2-yl) oxy)pentan-2-ol | 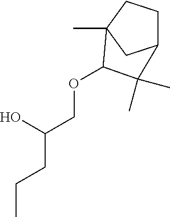 [SMILES: CCCC(O)COC1C2(C)CCC(C2)C1(C)C] | 5 | / | Synthesis given in Example 1 |
| 1-((1,3,3- trimethylbicyclo[2.2.1] heptan-2-yl)oxy) pentan-2-one | 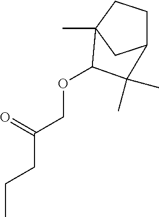 [SMILES: CCCC(=O)COC1C2(C)CCC(C2)C1(C)C] | 6 | / | Synthesis given in Example 1 |
| 1-((1,3,3,- trimethylbicyclo[2.2.1] heptan-2-yl) oxy)butan-2-ol | 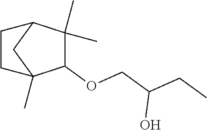 [SMILES: CCC(O)COC1C2(C)CCC(C2)C1(C)C] | 7 | / | Synthesis given in Example 1 |
| DL-Patchenol | 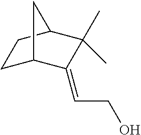 | 8 | 2226-05-3 | Sigma Aldrich |
| Belambre | 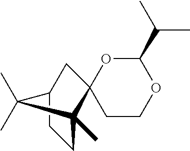 | 9 | 188199-50-0 | EP0761664 US2008161223 |
| 1′,2,5(S),7′,7′-Pentamethyl- spiro[1,3-dioxane-4,2′- bicyclo[2.2.1]heptane] | 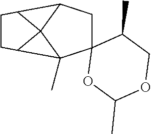 [SMILES: CC1OC[C@H](C)C2(CC3C4CCC5C43C52C)O1] | 10 | / | US2008161223 |
| 1′,2,5,7′,7′-Pentamethylspiro [1,3-dioxane-4,2′- bicyclo[2.2.1]heptane] | 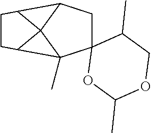 [SMILES: CC1OCC(C)C2(CC3C4CCC5C43C52C)O1] | 11 | / | US2008161223 |
| Spirambrene | 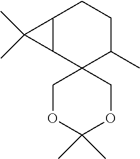 | 12 | 121251-67-0 | BOC Sciences |
| 2,4,4,10,10-Pentamethyl-8- propan-2-yl-3,5- dioxatricyclo[6.2.1.02,6] undecane | 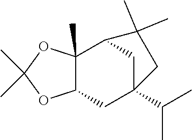 [SMILES: CC(C)[C@]12C[C@@H]3OC(C)(C)O[C@]3(C)[C@H](C1)C(C)(C)C2] | 13 | / | US2008161223 |
| 1-((2,2,6- trimethylcyclohexyl)oxy) pentan-2-ol | 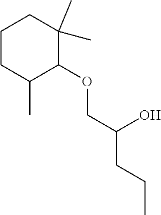 [SMILES: CCCC(O)COC1C(C)CCCC1(C)C] | 14 | 87887-12-5 | Synthesis given in Example 1 |
| 1-((2,2,6- trimethylcyclohexyl)oxy) butan-2-ol | 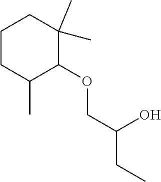 | 15 | 87887-13-6 | Synthesis given in Example 1 |
| Ambermax | 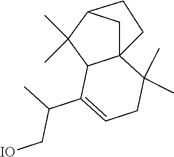 | 16 | 929625-08-1/ 1001252- 30-7 | Pell Wall |
| | 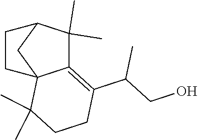 [SMILES: CC(CO)C1=C2C(C)(C)C3CCC2(C3)C(C)(C)CC1] | | | |
| Amberketal | 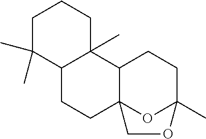 | 17 | 57345-19-4 | Chemos GmbH |
| Ambroxan or (−)-Ambroxide | 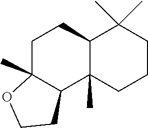 [SMILES: CC1(C)CCC[C@]2(C)[C@H]3CCO[C@]3(C)CC[C@@H]12] | 18 | 6790-58-5 | Pell Wall |
| Amboryl acetate | 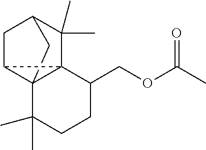 | 19 | 59056-62-1 | Creating Perfume |
| 6-Ethyl-2,2,10,10- tetramethyl- 5,7-dioxatetracyclo [9.2.1.01,9.04,8] tetradecane | 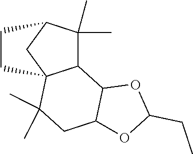 [SMILES: CCC1OC2CC(C)(C)[C@@]34CC[C@@H](C3)C(C)(C)C4C2O1] | 20 | / | WO201000083 |
| 5-(1-Oxopropan- 2-yl)isolongifol- 5-ene | 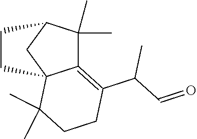 [SMILES: CC(C=O)C1=C2C(C)(C)[C@H]3CC[C@@]2(C3)C(C)(C)CC1] | 21 | / | WO2007030963 |
| 5-(3-Hydroxybutan- 2-yl)isolongifol- 5-ene | 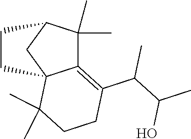 [SMILES: CC(O)C(C)C1=C2C(C)(C)[C@H]3CC[C@@]2(C3)C(C)(C)CC1] | 22 | / | WO2007030963 |
| 2,2,6,10,10- Pentamethyl-5,7- dioxatetracyclo [9.2.1.01,9.04,8] tetradecane | 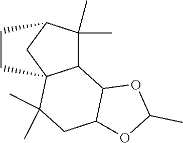 [SMILES: CC1OC2CC(C)(C)[C@@]34CC[C@@H](C3)C(C)(C)C4C2O1] | 23 | / | WO201000083 |
| 2,2,10,10- Tetramethyl-5,7- dioxatetracyclo [9.2.1.01,9.04,8] tetradecane | 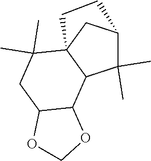 | 24 | / | WO201000083 |
| 2-(2,2,7,7- Tetramethyl-5- tricyclo[6.2.1.01,6] undec-5- enyl)propan-1-ol | 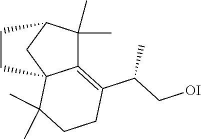 | 25 | / | WO2007030963 |
| 5-(1-Methoxypropan- 2-yl)-2,2,7,7- tetramethyltricyclo [6.2.1.01,6] undec-5-ene | 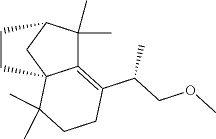 [SMILES: COC[C@@H](C)C1=C2C(C)(C)[C@H]3CC[C@@]2(C3)C(C)(C)CC1] | 26 | / | WO2007030963 |
| 5-(1-Hydroxybutan- 2-yl)isolongifol- 5-ene | 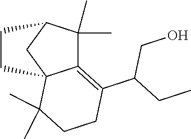 [SMILES: CCC(CO)C1=C2C(C)(C)[C@H]3CC[C@@]2(C3)C(C)(C)CC1] | 27 | / | WO2007030963 |
| 2-(2,2,7,7- Tetramethyl-5- tricyclo[6.2.1.01,6] undec-4- enyl)butan-1-ol | 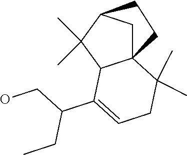 [SMILES: CCC(CO)C1=CCC(C)(C)[C@@]23CC[C@@H](C2)C(C)(C)C13] | 28 | / | WO2007030963 |
| | 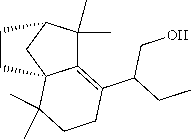 [SMILES: CCC(CO)C1=C2C(C)(C)[C@H]3CC[C@@]2(C3)C(C)(C)CC1] | | | |
| 5-(1-Methoxybutan- 2-yl)-2,2,7,7- tetramethyltricyclo [6.2.1.01,6] undec-5-ene | 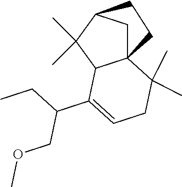 [SMILES: CCC(COC)C1=CCC(C)(C)[C@@]23CC[C@@H](C2)C(C)(C)C13] | 29 | / | WO2007030963 |
| | 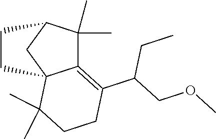 [SMILES: CCC(COC)C1=C2C(C)(C)[C@H]3CC[C@@]2(C3)C(C)(C)CC1] | | | |
| 5-(1- Methoxybutan-2-yl) isolongifol-5-ene | 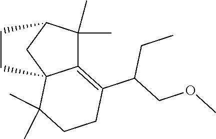 [SMILES: CCC(COC)C1=C2C(C)(C)[C@H]3CC[C@@]2(C3)C(C)(C)CC1] | 30 | / | WO2007030963 |
| 5-(1-methoxybutan-2-yl) isolongifol-4-ene | 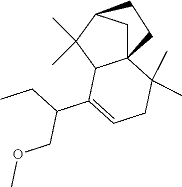 [SMILES: CCC(COC)C1=CCC(C)(C)[C@@]23CC[C@@H](C2)C(C)(C)C13] | 31 | / | WO2007030963 |
| (1S,8S)-2,2,7,7- Tetramethyltricyclo [6.2.1.01,6] undecan-5-one | 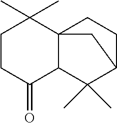 [SMILES: CC1(C)C2CCC3(C2)C1C(=O)CCC3(C)C] | 32 | 29461-14-1 | Biosynth |
| Sylvamber | 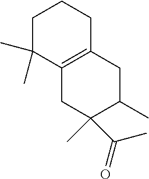 | 34 | 54464-57-2 | Pell Wall |
| 3′,4,4,8,8a- Pentamethylspiro[2,3,4a, 5,6,8-hexahydro- 1H-naphthalene-7,2′- oxirane] | 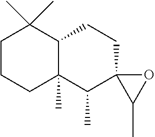 [SMILES: CC1O[C@@]12CC[C@H]1C(C)(C)CCC[C@]1(C)[C@H]2C] | 35 | / | WO2012069647 |
| 3′,4,4,8a- Tetramethylspiro[2,3,4a, 5,6,8-hexahydro- 1H-naphthalene-7,2′- oxirane] | 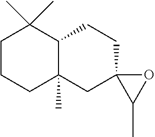 [SMILES: CC1O[C@@]12CC[C@H]1C(C)(C)CCC[C@]1(C)C2] | 36 | / | WO2012069647 |
| 4,4,8,8a- Tetramethylspiro[2,3,4a, 5,6,8-hexahydro- 1H-naphthalene-7,2′- oxirane] | 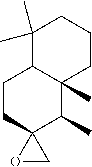 | 37 | / | WO2012069647 |
| 4,4,8a- Trimethylspiro[2,3,4a,5, 6,8-hexahydro- 1H-naphthalene-7,2′- oxirane] | 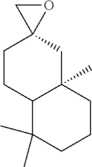 | 38 | / | Helv. Chim. Acta, 2004, 87, 1616 |
| 5-(1-Hydroxypropan-2- yl)isolongifolane | 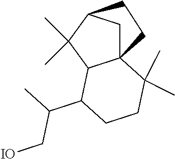 | 39 | / | WO2007030963 |
| Boisambrene | 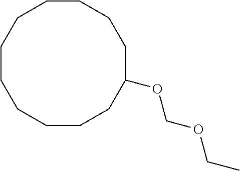 [SMILES: CCOCOC1CCCCCCCCCCC1] | 40 | 58567-11-6 | Pell Wall |
| Hydroxyambran | 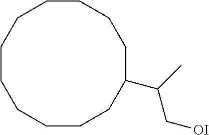 | 41 | 118562-73-5 | Pell Wall |
| Cyclisone/Trimofix | 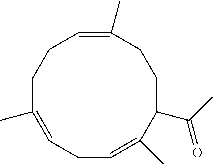 [SMILES: CC(=O)C1CC/C(C)=C\CC/C(C)=C\C/C=C\1C] | 42 | 28371-99-5 | Augustus Oils |
| Cedroxyde | 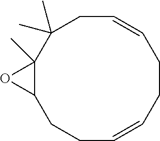 [SMILES: CC1(C)C/C=C\CC/C=C\CCC2OC21C] | 43 | 71735-79-0 | Perfumer Supply house LLC |
| (E)-woody epoxide | 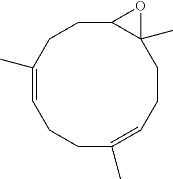 | 44 | 13786-79-3 | Boc Sciences |
| 5α-Androst- 16-en-3-one | 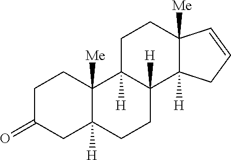 | 45 | 18339-16-7 | Carbosynth |
| Androstadienol | 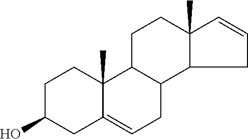 [SMILES: C[C@@]12C=CCC1C1CC=C3C[C@@H](O)CC[C@]3(C)C1CC2] | 46 | 1224-94-8 | Steraloids Inc |

Dose-response analyses using the luciferase-based reporter assay were performed using semi-logarithmic serial dilutions of the compounds, from 1 mM to 316 nM, on OR7C1. 46 compounds were tested in concentration-response analyses. In each experiment, an empty vector (pEFIBRHO) was used as negative control. Full results including the calculated log EC₅₀ are given in Table 2. Values are given as the negative logarithm of the EC₅₀ in Moles.

TABLE 2
Compound

| Name | Number | -LogEC₅₀ (average n > 3) |
|---|---|---|
| Norlimbanol | 1 | 5.4 |
| Ambercore | 2 | 4.9 |
| Grisalva | 3 | 4.6 |
| Georgywood | 4 | 4.6 |
| 1-((1,3,3-trimethylbicyclo[2.2.1]heptan-2-yl)oxy)pentan-2-ol | 5 | 4.3 |
| 1-((1,3,3-trimethylbicyclo[2.2.1]heptan-2-yl)oxy)pentan-2-one | 6 | 4.3 |
| 1-((1,3,3-trimethylbicyclo[2.2.1]heptan-2-yl)oxy)butan-2-ol | 7 | 4.3 |
| DL-Patchenol | 8 | 4.5 |
| Belambre | 9 | 5.8 |
| 1′,2,5,7′,7′-Pentamethylspiro[1,3-dioxane-4,2′- | 10 | 5.6 |
| bicyclo[2.2.1]heptane] | | |
| 1′,2,5,7′,7′-Pentamethylspiro[1,3-dioxane-4,2′- | 11 | 5.7 |
| bicyclo[2.2.1]heptane] | | |
| Spirambrene | 12 | 4.9 |
| 2,4,4,10,10-Pentamethyl-8-propan-2-yl-3,5- | 13 | 6.4 |
| dioxatricyclo[6.2.1.02,6]undecane | | |
| 1-((2,2,6-trimethylcyclohexyl)oxy)pentan-2-ol | 14 | 3.9 |
| 1-((2,2,6-trimethylcyclohexyl)oxy)butan-2-ol | 15 | 3.9 |
| Ambermax | 16 | > 8 |
| Amberketal | 17 | 5.6 |
| Ambroxan | 18 | 4.5 |
| Amboryl acetate | 19 | 4.2 |
| 6-Ethyl-2,2,10,10-tetramethyl-5,7- | 20 | 4.9 |
| dioxatetracyclo[9.2.1.01,9.04,8]tetradecane | | |
| 5-(1-Oxopropan-2-yl)isolongifol-5-ene | 21 | 6.3 |
| 5-(3-Hydroxybutan-2-yl)isolongifol-5-ene | 22 | 5.8 |
| 2,2,6,10,10-Pentamethyl-5,7- | 23 | 5.3 |
| dioxatetracyclo[9.2.1.01,9.04,8]tetradecane | | |
| 2,2,10,10-Tetramethyl-5,7- | 24 | 5.0 |
| dioxatetracyclo[9.2.1.01,9.04,8]tetradecane | | |
| 2-(2,2,7,7-Tetramethyl-5-tricyclo[6.2.1.01,6]undec-5- | 25 | 4.3 |
| enyl)propan-1-ol | | |
| 5-(1-Methoxypropan-2-yl)-2,2,7,7- | 26 | 5.6 |
| tetramethyltricyclo[6.2.1.01,6]undec-5-ene | | |
| 5-(1-Hydroxybutan-2-yl)isolongifol-5-ene | 27 | 5.5 |
| 2-(2,2,7,7-Tetramethyl-5-tricyclo[6.2.1.01,6]undec-4- | 28 | 6.6 |
| enyl)butan-1-ol | | |
| 5-(1-Methoxybutan-2-yl)-2,2,7,7- | 29 | 5.2 |
| tetramethyltricyclo[6.2.1.01,6]undec-5-ene | | |
| 5-(1-Methoxybutan-2-yl)isolongifol-5-ene | 30 | 5.6 |
| 5-(1-methoxybutan-2-yl)isolongifol-4-ene | 31 | 5.4 |
| (1S,8S)-2,2,7,7-Tetramethyltricyclo[6.2.1.01,6]undecan-5-one | 32 | 4.7 |
| Sylvamber | 34 | 4.6 |
| 3′,4,4,8,8a-Pentamethylspiro[2,3,4a,5,6,8-hexahydro- | 35 | 4.5 |
| 1H-naphthalene-7,2′-oxirane] | | |
| 3′,4,4,8a-Tetramethylspiro[2,3,4a,5,6,8-hexahydro-1H- | 36 | 4.7 |
| naphthalene-7,2′-oxiranel | | |
| 4,4,8,8a-Tetramethylspiro[2,3,4a,5,6,8-hexahydro-1H- | 37 | 3.3 |
| naphthalene-7,2′-oxirane] | | |
| 4,4,8a-Trimethylspiro[2,3,4a,5,6,8-hexahydro-1H- | 38 | 4.3 |
| naphthalene-7,2′-oxirane] | | |
| 5-(1-Hydroxypropan-2-yl)isolongifolane | 39 | 5.8 |
| Boisambrene | 40 | 4.3 |
| Hydroxyambran | 41 | 5.5 |
| Cyclisone / Trimofix | 42 | 4.7 |
| CEDROXYDE | 43 | 5.7 |
| (E)-woody epoxide | 44 | 5.1 |
| 5a-Androst-16-en-3-one | 45 | 4.7 |
| Androstadienol | 46 | 4.9 |

From the results shown in Table 2, it can be observed that the tested compounds belonging to compounds of Formula I, II, III and IV were able to activate OR7C1 in a luciferase-based reporter assay. This receptor was responding with an average log EC50 close to, 4.85 for Structure (1), to 5.045 and 5.06, for compounds belonging to compounds of Formula structure (II), (III) and to 4.78 for compounds belonging to compounds of Formula (IV) respectively. Furthermore, Table 2 indicates that the tested compounds specifically activate olfactory receptors OR7C1.

These results indicate that compounds belonging to compounds of Formula I, II, III and IV are involved in an increase of cAMP in cells expressing OR7C1 and constitute valuable candidates to activate ectopic OR7C1. Therefore, treating OR7C1 expressing cells with compounds for use in the invention will induce an increase in cAMP levels in these cells.

### Example 3: OR7C1 is Expressed Ectopically in Primary Culture of Melanocytes

Using public databases (https://www.ebi.ac.uk/gxa/home; https://www.proteinatlas.org/), the inventors have found that melanocytes expressed the olfactory receptor of interest namely OR7C 1 thereby showing the endogenous expression of this receptor outside the olfactory epithelium.

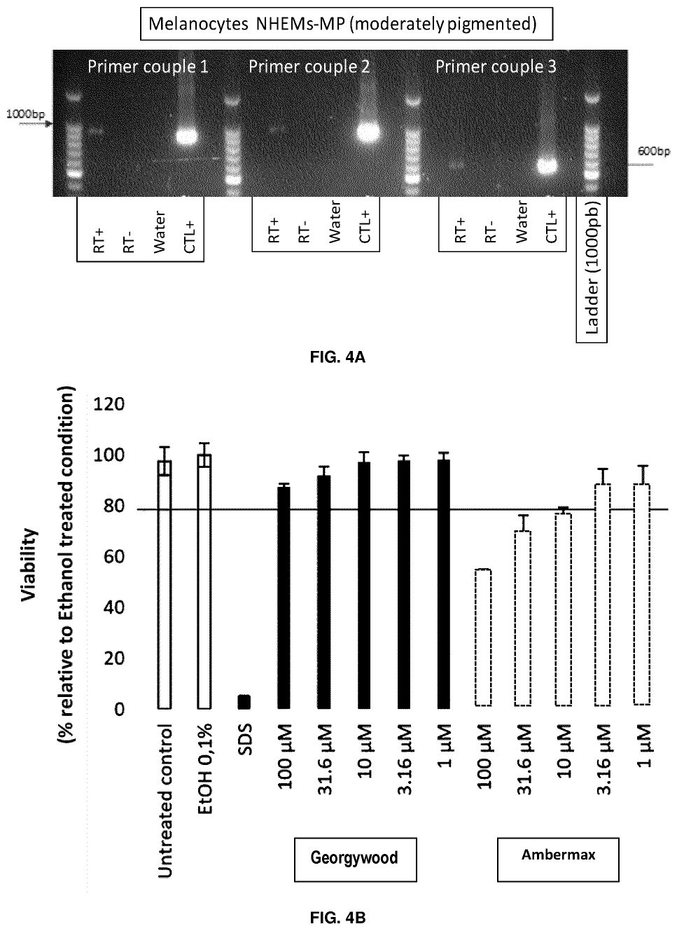

This was confirmed by PCR. OR7C1 expression levels: primary neonatal foreskins melanocytes moderately pigmented (NHEMs-MP) were grown as described herein before. After total mRNA extraction and a cDNA synthesis step (RT), PCR experiments were performed using specific primers for the detection/amplification of OR7C1. As observed in FIG. 4A, a PCR product for OR7C1 cDNA was detected in the RT+ sample (corresponding to the sample where the mRNA of NHEMs-MP was reverse transcripted into cDNA). The RT− sample (sample with no step of cDNA synthesis) showed no amplification of the target gene and was used, like water, as negative control. The CTL+ lane, corresponded to the amplification obtained after PCR using a plasmid containing cDNA sequence of OR7C1 (positive control). The data observed indicated that, using 3 different couples of primers, melanocytes ectopically expressed the olfactory receptor OR7C1 as described in the different databases (FIG. 4A). Therefore, melanocytes can be used as cellular model to test the effects of compounds according to embodiments of the invention on melanogenesis, a pathway sensitive to the variations of cAMP levels present in these cells.

An MTS assay (3-(4,5-dimethythiazol-2-yl)-5-(3-carboxymethoxyphenyl)-2-(4-sulfophenyl)-2H-tetrazolium) was performed to measure the maximal concentrations the compounds which can be used in the culture medium of NHEMs-MP without affecting cell viability. The assay was performed as described hereinabove. The different tested concentrations were based on the EC50 (in Mole) obtained in the luciferase-based reporter assay (Table 2). After 4 days of contact, an MTS assay was performed on the treated cells with the test compounds (5 concentrations) or with the cytotoxic reference; the experiment was performed in triplicate (n=3). Results are presented in FIG. 4B. Cell viability was analyzed, and toxicity/viability was expressed in percentage relative to the ethanol treated condition which was fixed at 100%. The cytotoxicity threshold was set by convention at 80% of viability. As shown in FIG. 4B, Georgywood showed no significant cell toxicity independently of the tested concentration. After 4 days of treatment, the decrease of viability appeared at concentrations equal or greater than 10 μM.

These results indicate that OR7C1 is expressed in cells outside the olfactory epithelium. This receptor is present in melanocytes where melanogenesis occurs. Compounds according to embodiments of the invention were well tolerated, can be used to modulate the cAMP in these cells.

### Example 4: Effects of Formulation on Human Melanocytes Melanogenesis

The effects of the compounds according to embodiments of the invention (Amberketal and Ambermax) on melanogenesis was tested by treating Normal Human Epidermal Melanocytes (NHEMs-MP) with said compounds.

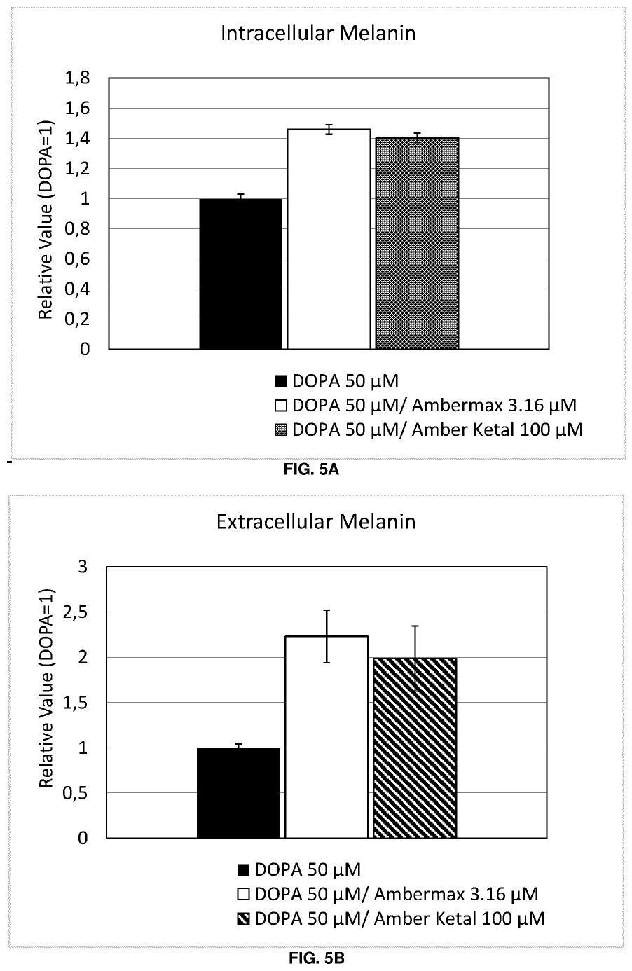

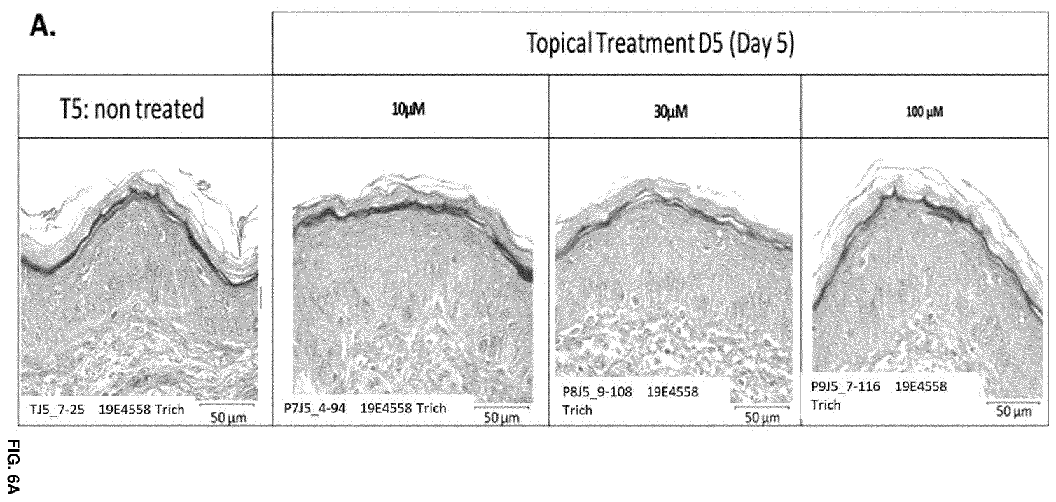

The effects of these 2 compounds, namely Ambermax and Amberketal, on the melanin production were evaluated as described above. Based on MTS results, moderately pigmented (MP) NHEMs were treated with these 2 compounds at one concentration (100 μM for Amberketal and 3.16 μM for Ambermax) for 4 days. In parallel, cell cultures were also treated with Ethanol 0.1%, a concentration corresponding to the test compounds solvent, and with a known modulator of pigmentation (L-DOPA 50 μM). Microscopical analyses showed an accumulation of melanin in the cytoplasm of the cells in the treated conditions (not shown). At the end of the treatments (D4), cells were lysed, the melanin content was quantified by spectrophotometry and normalized to the amount of proteins. Extracellular melanin was also evaluated by the same methodology after centrifugation of the culture media. As expected, L-DOPA enhanced slightly the melanin content in NHEMs after the 4 days of treatment. The results of the experiment are shown in FIGS. 5A and 5B. It can be seen that the tested compounds, Amberketal (100 μM) and Ambermax (3.16 μM), increased in a highly significant way the intracellular melanin content (+40%; p<0.01**; n=12 and +46%; p<0.01**; n=12, respectively) as well as the melanin present in the media (+99%; p<0.01**; n=10 and +123%; p<0.01** n=10; respectively) in comparison with the treatment DOPA (50 μM) (FIGS. 5A and 5B. Results are expressed as relative values; DOPA value=1).

In melanocytes, cells that endogenously expressed OR7C1, one of the major cAMP dependent pathway is melanogenesis. The results indicate that treatments with compounds according to embodiments of the invention were able to modulate melanin production.

### Example 5: Macroscopic Effects of Formulation on Human Explants Melanogenesis

The effects ex vivo was shown by treating human biopsies for 10 days (human living explants: 12 mm+/−1, Caucasian donor, skin type III) with a systemic application, intradermic injections or a topical application of compounds according to embodiments of the invention (Ambermax). Concentrations of 1, 5 and 10 μM of this compound were used for systemic application and intradermic injections on the human explants. For topical application, biopsies were treated with 10, 30, and 100 μM of Ambermax. As control, non-treated explants or/and explants exposed to chronic UVA-irradiations were used as described in the experimental procedures.

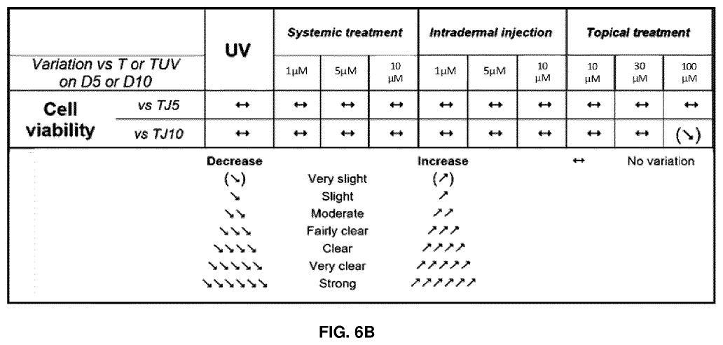

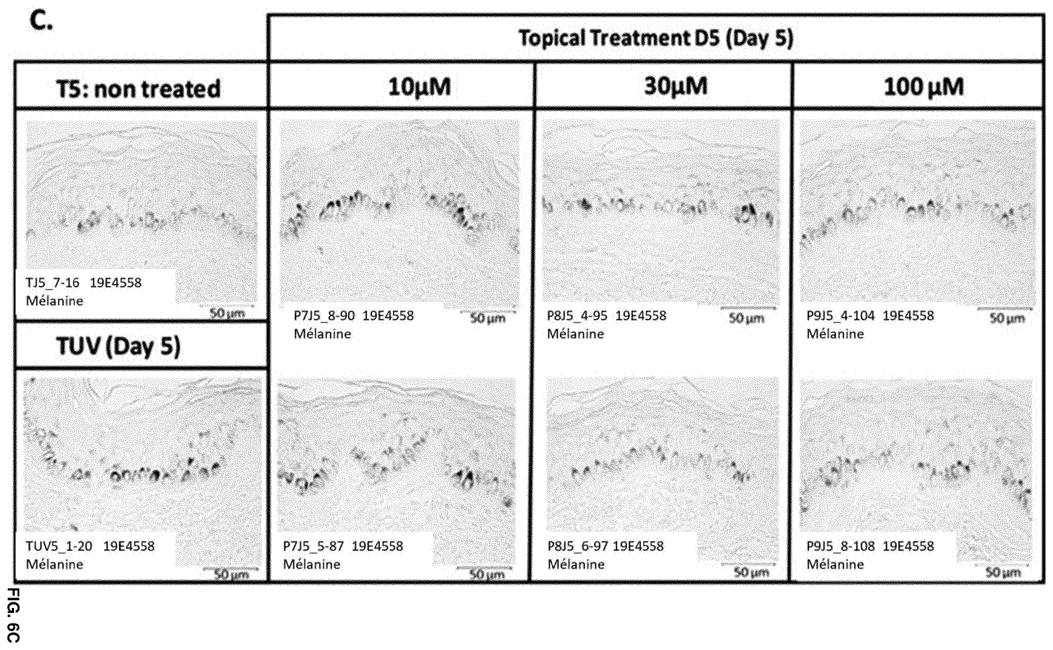

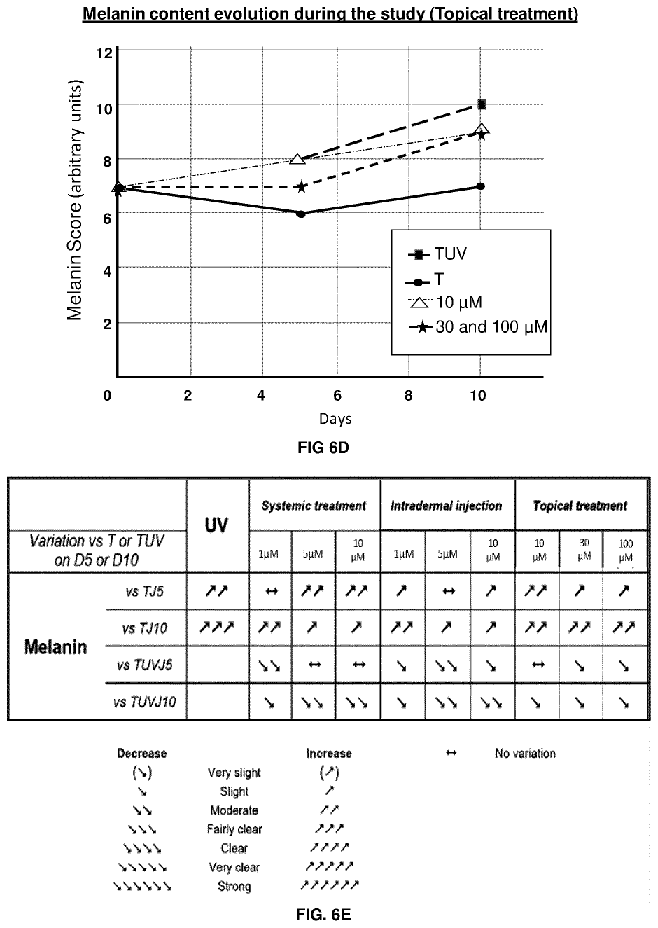

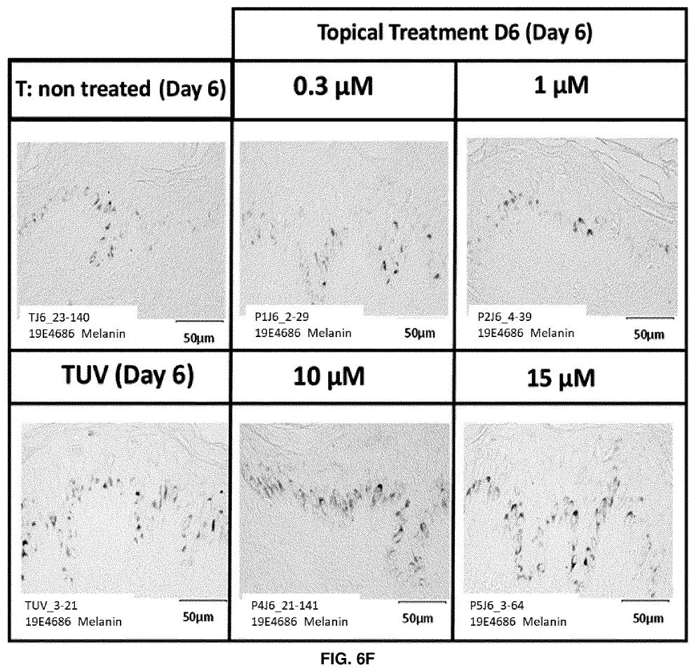

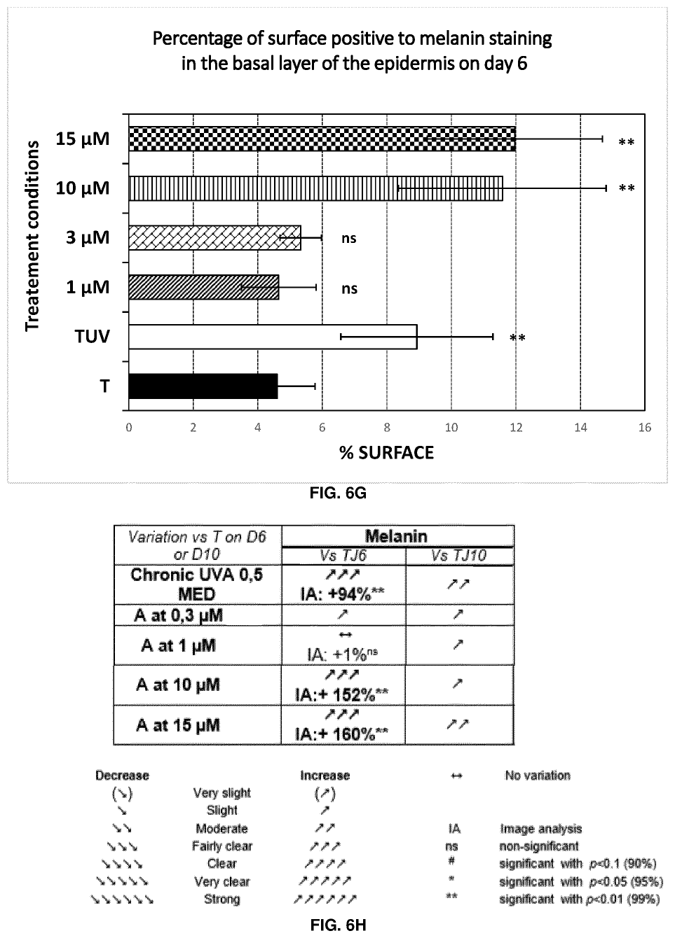

As shown in FIG. 6B, at the starting point of the study (D0), the cell viability of explants was normal with a typical organization of the papillary dermal and epidermal structures. At day 5 (D5) and day 10 (D10), treatments performed with Ambermax at 3 different concentrations and with 3 different methods of application demonstrate no significant alterations of explant viability except for the topical administration with highest concentration (100 μM). At this concentration, the macroscopic evaluation of the explant showed a very slight decrease in the viability (very slightly altered, VSA) in the epidermis, but nevertheless no modification of the papillary dermis where melanogenesis occurs was observed. In all the other conditions, the dermal and epidermal structures were similar to the non-treated explants. Representative pictures of explant viability after 5 days of topical treatment are shown in FIG. 6A. In Chronic UVA-irradiations condition, no alteration of epidermal compartment was observed (positive control, FIG. 6B). Altogether, these results indicate that compounds according to embodiment of the invention are well tolerated and can be used to stimulate melanogenesis in an ex vivo model like skin biopsies. A summary of the macroscopic effects of Ambermax on explant viability is shown in FIG. 6B.

The effect of compounds according to embodiments of the invention on melanogenesis was measured using Masson's Fontana staining on paraffin skin sections as described in experimental procedures at D0, D5 and D10. After 5 days, a systemic treatment (3 and 5 μM) with the test compound induced a rise of melanogenesis equivalent to the one obtained with a chronic UVA irradiation. At day 10, 1 μM of Ambermax was shown to be the most efficient concentration to stimulate melanin synthesis. After 5 days of intradermal injection, Ambermax was shown to induce an increase of melanogenesis slightly weaker than that achieved with chronic UVA. This activity was observed with 1 μM and 10 μM concentrations. After intradermal injection for 10 days, the most effective concentration was 1 μM, a little more efficient than 5 μM and 10 μM concentrations. Topical treatment, for 5 days, with Ambermax induces an increase in melanogenesis comparable to that obtained with chronic UVA irradiation (FIGS. 6C, 6D). This activity was observed with 10 μM concentration. After topical treatment for 10 days, the concentrations 10 μM, 30 μM and 100 μM concentrations showed a moderate melanogenesis induction, slightly lower than that obtained with of chronic UVA (FIG. 6D). A summary of the macroscopic effects of compound of invention on melanogenesis is present in FIG. 6E.

Further experiments were performed on a second batch of human skin explants coming from an abdoplasty of Caucasian woman (phototype II). Results are represented in FIGS. 6F, 6G and 6H. It can be seen that Ambermax induced an increase of the melanin content present in the basal layer in skin explants. FIG. 6F shows representative pictures of skin treated with Ambermax 0.3 μM, 1 μM, 10 μM and 30 μM at D6. Quantification of the percentage of surface positive to melanin in the basal layer of the epidermis at the different concentrations was performed using the image analysis procedure mentioned hereinabove. At D6, in non-treated explants (T), it can be seen that the melanin content represented 4.6% of the surface of the basal layer of the epidermis (FIG. 6G). The UVA-irradiations exposure (TUVJ6) induced a significant increase of 94% (**; p<0.01) of the melanin content in the basal layer, compared to the non-treated explants (TJ6). After quantification, it can be seen that at 10 μM or 15 μM, Ambermax induced an increase of melanin content at microscopical level (FIG. 6F). Image analysis method showed that it corresponds to a significant increase of 152% and 160% respectively (**, p<0.01, FIG. 6H). A summary of the macroscopic effects of Ambermax (D6 and D10) on melanogenesis and results of image analyses is shown in FIG. 6H.

Ex vivo experiments (on human living skin explants) with different concentrations of Ambermax (0.3-100 μM) and 3 different ways of application showed that this compound is well tolerate and doesn't induce explant toxicity. A very slight alterations was yet observed after application of 100 μM on D10 but they had no impact on either the product activity or the melanin synthesis. These modifications would probably not be observed on an in vivo model. Moreover, microscopical observation of and semi-quantification of melanin staining present in FFPE skin sections by image analysis demonstrate that the Ambermax is very effective to enhance melanogenesis without UVs exposure. S

1. A method of self-tanning, comprising applying onto a desired skin surface a self-tanning substance selected from the group comprising a compound of formula (IIb), (IIa), (IIIa), (IV), (Ie), (If), (Ig), (Ic), or (Id), a racemate, a stereoisomer, a tautomer, a salt, and/or a solvate thereof, including mixtures thereof, 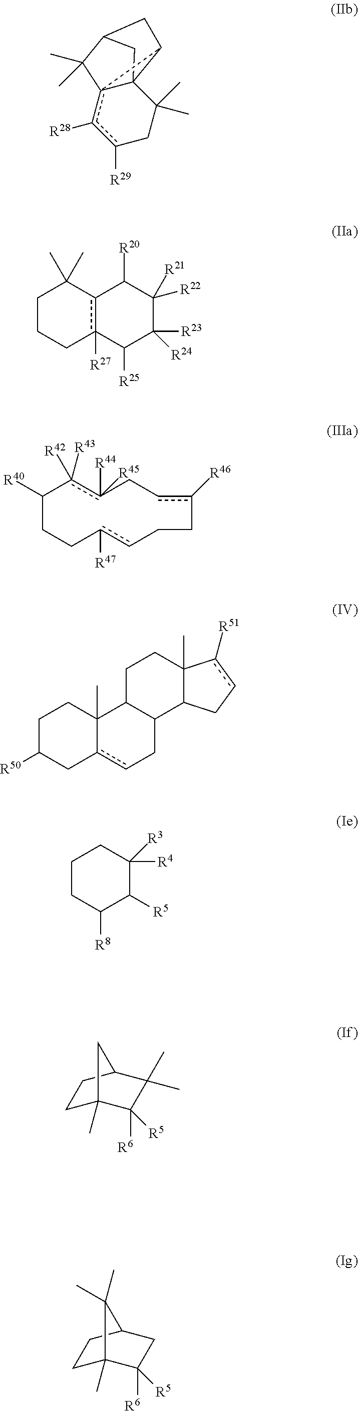 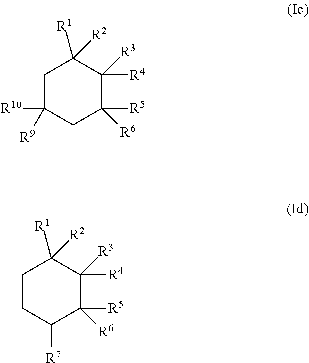
wherein the dash bond [symbol] represents an optional double bond; and wherein [symbol] represent an optional single bond; and
R²⁸ is selected from the group comprising hydrogen, oxo, C₁₋₆alkyl, —O—C₁₋₆alkyl, —[CR¹⁷R¹⁸]ₘ—O—CO—C₁₋₆alkyl, —[CR¹⁷R¹⁸]ₘ—O—C₁₋₆alkyl, —[CR¹⁷R¹⁸]ₘ—COH, and —[CR¹⁷R¹⁸]m OH; m is an integer selected from 1, 2, 3, 4 or 5,
R¹⁷, R¹⁸, are each independently selected from hydrogen or C₁₋₆alkyl;
R²⁹ is hydrogen or C₁₋₆alkyl;
or R²⁹ and R²⁸ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2;
R²⁰ is hydrogen or C₁₋₆alkyl;
R²¹ is hydrogen or C₁₋₆alkyl;
R²² is selected from the group comprising hydrogen, C₁₋₆alkyl, and —CO—R¹⁹;
R²³ is hydrogen or C₁₋₆alkyl;
R²⁴ is hydrogen or C₁₋₆alkyl;
or R²³ and R²⁴ together with the carbon atom to which they are attached form a 3- to 6-membered heterocyclyl moiety; wherein said heterocyclyl is optionally substituted with one or more substituents each independently selected from C₁₋₆alkyl;
R²⁵ is hydrogen or C₁₋₆alkyl;
or R²⁴ and R²⁵ form together with the carbon atom to which they are attached a 5 or 6-membered heterocyclyl moiety, wherein said heterocyclyl is optionally substituted with one or more substituents each independently selected from C₁₋₆alkyl, or wherein one of said substituent and R²³ form together with the atoms to which they are attached a 3- to 6-membered heterocyclyl moiety;
R²⁷ if present is hydrogen or C₁₋₆alkyl;
or R²⁰ and R²⁷ together with the carbon atoms to which they are attached form a C3-membered ring;
R⁴⁰ is selected from the group comprising —[CR¹⁷R¹⁸]ₘ—OH, —CO—C₁₋₆alkyl, —O—([CR¹⁷R¹⁸]ₘ—O—)ₜ—C₁₋₆alkyl, t is an integer selected from 0, 1, 2, or 3;
R⁴², R⁴³, R⁴⁴, R⁴⁵, R⁴⁶, R⁴⁷ are each independently selected from hydrogen or C₁₋₆alkyl;
or R⁴⁰ and R⁴² together with the carbon atom to which they are attached form a 3- to 6-membered heterocyclyl moiety;
R⁵⁰ is selected from OH or oxo;
R⁵¹ is hydrogen or C₁₋₄alkyl;
R¹, R² are each independently selected from hydrogen or C₁₋₆alkyl;
R³, R⁴ are each independently selected from hydrogen or C₁₋₆alkyl;
or R¹ with R³ together with the carbon atoms to which they are attached form a saturated C3 to C5-membered ring, wherein said ring is optionally substituted with one or more C₁₋₆alkyl substituents;
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —X—[CR¹³R¹⁴]ₙ—O—[CR¹⁷R¹⁸]ₘ—OH, —([CR¹⁷R¹⁸]ₘ—O)_{y}—C₁₋₆alkyl, —[CR¹³R¹⁴]ₙ—CO—R¹⁹;
m is an integer selected from 1, 2, 3, 4 or 5, n is an integer selected from 0, 1, 2, 3 or 4; y is an integer selected from 1, 2 or 3;
X is a single bond or O,
R¹³, R¹⁴, R¹⁶, R¹⁷, R¹⁸, R¹⁹ are each independently selected from hydrogen or C₁₋₆alkyl;
R¹⁵ is OH, or R¹⁵ together with R¹⁶ form an oxo group;
or R³ and R^{s} together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2;
R⁶ is hydrogen or C₁₋₆alkyl;
or R⁵ and R⁶ form together ═[CR¹⁴]—[CR¹⁷R¹⁸]ₘ—OH; or R⁵ and R⁶ together with the carbon atom to which they are attached form a 5- or 6-membered heterocyclyl moiety, wherein said heterocyclyl is optionally substituted with one or more C₁₋₆alkyl substituents;
R⁷ is hydrogen or C₁₋₆alkyl;
R⁸ is hydrogen or C₁₋₆alkyl;
or R^{s} and R⁸ together with the carbon atoms to which they are attached form a saturated or unsaturated C5 or C6-membered ring, wherein said ring is optionally substituted with one or more substituents each independently selected from oxo, C₁₋₆alkyl, —O—C₁₋₆alkyl, —CO—C₁₋₆ alkyl, —[CR¹⁷R¹⁸]ₘ—O—CO—C₁₋₆alkyl, —([CR¹⁷R¹⁸]ₘ—O)_{y}—C₁₋₆alkyl, —[CR¹⁷R¹⁸]ₘ—COH, or —[CR¹⁷R¹⁸]ₘ—OH; or two substituents on said ring form together with the carbon atoms to which they are attached a 3- to 6-membered heterocyclyl moiety, wherein said heterocyclyl moiety is optionally substituted with one or more substituents each independently selected from C₁₋₆alkyl, or two substituents on said heterocyclyl form together with the atoms to which they are attached a 3- to 6-membered heterocyclyl moiety;
R⁹, R¹⁰ are each independently selected from hydrogen or C₁₋₆alkyl;
or R⁶ and R⁹ together with the carbon atoms to which they are attached form a C3-membered ring;
or R¹ with R⁹ together with the carbon atoms to which they are attached form a saturated C5-membered ring, wherein said ring is optionally substituted with one or more C₁₋₆alkyl substituents;
or R³ with R⁹ together with the carbon atoms to which they are attached form a saturated C5-membered ring, wherein said ring is optionally substituted with one or more C₁₋₆alkyl substituents.

2. A compound of formula (Ih), or a racemate, a stereoisomer, a tautomer, a salt, and/or a solvate thereof, including mixtures thereof; 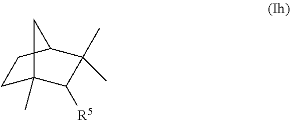
wherein
R⁵ is selected from the group comprising —O—CHR¹⁴—C(═O)—[CR¹⁷R¹⁸]ₘ—R¹⁹, —O—CHR¹⁴—CH(OH)—[CR¹⁷R¹⁸]ₘ—R¹⁹;
m is an integer selected from 1, 2, 3, 4 or 5;
R¹⁴, R¹⁷, R¹⁸, R¹⁹ are each independently selected from hydrogen or C₁₋₆alkyl.

3. The method according to claim 1, wherein the compound comprises a compound of formula (Ih), or a racemate, a stereoisomer, a tautomer, a salt, and/or a solvate thereof, including mixtures thereof: 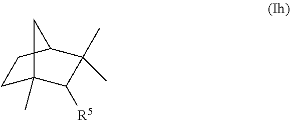
wherein
R⁵ is selected from the group comprising —O—CHR¹⁴—C(═O)—[CR¹⁷R¹⁸]ₘ—R¹⁹, —O—CHR¹⁴—CH(OH)—[CR¹⁷R¹⁸]ₘ—R¹⁹;
m is an integer selected from 1, 2, 3, 4 or 5;
R¹⁴, R¹⁷, R¹⁸, R¹⁹ are each independently selected from hydrogen or C₁₋₆alkyl.

4. The method according to claim 1, wherein the self-tanning comprises increasing the amount of melanin in melanocytes, increasing melanin synthesis, improving melanin transport and/or improving the distribution of melanin in suprabasal layers of the desired skin.

5. The method according to claim 1, wherein the method results in darkening the desired skin.

6. The method according to claim 1, wherein the method results in sunless tanning of the desired skin.

7. The method according to claim 1, wherein the compound is of formula (Ie), 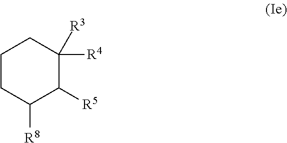
wherein
R³, R⁴ are each independently selected from hydrogen or C₁₋₆alkyl;
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —CO—R¹⁹;
m is an integer selected from 1, 2, 3, 4 or 5, n is an integer selected from 0, 1, 2, 3 or 4;
X is a single bond or O,
R¹³, R¹⁴, R¹⁶, R¹⁷, R¹⁸, R¹⁹ are each independently selected from hydrogen or C₁₋₆alkyl;
R¹⁵ is OH, or R¹⁵ together with R¹⁶ form an oxo group; and
R⁸ is hydrogen or C₁₋₆alkyl.

8. The method according to claim 1, wherein the compound is of formula (If), 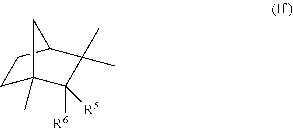
wherein
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —CO—R¹⁹;
m is an integer selected from 1, 2, 3, 4 or 5, n is an integer selected from 0, 1, 2, 3 or 4;
X is a single bond or O,
R¹³, R¹⁴, R¹⁶, R¹⁷, R¹⁸, R¹⁹ are each independently selected from hydrogen or C₁₋₆alkyl;
R¹⁵ is OH, or R¹⁵ together with R¹⁶ form an oxo group; and
R⁶ is hydrogen or C₁₋₆alkyl;
or R⁵ and R⁶ form together ═[CR¹⁴]—[CR¹⁷R¹⁸]ₘ—OH.

9. The method according to claim 1, wherein the compound is of formula (Ig), 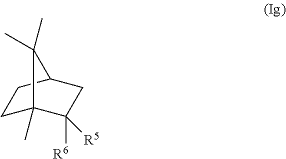
wherein
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —CO—R¹⁹;
m is an integer selected from 1, 2, 3, 4 or 5, n is an integer selected from 0, 1, 2, 3 or 4;
X is a single bond or O,
R¹³, R¹⁴, R¹⁶, R¹⁷, R¹⁸, R¹⁹ are each independently selected from hydrogen or C₁₋₆alkyl;
R¹⁵ is OH, or R¹⁵ together with R¹⁶ form an oxo group;
R⁶ is hydrogen or C₁₋₆alkyl;
or R⁵ and R⁶ form together ═[CR¹⁴]—[CR¹⁷R¹⁸]ₘ—OH; or R⁵ and R⁶ together with the carbon atom to which they are attached form a 5- or 6-membered heterocyclyl moiety, wherein said heterocyclyl is optionally substituted with one or more C₁₋₆alkyl substituents.

10. The method according to claim 1, wherein the compound is of formula (IIb), 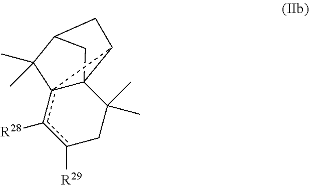
wherein the dash bond [symbol] represents an optional double bond; and wherein [symbol] represent an optional single bond;
R²⁸ is selected from the group comprising hydrogen, oxo, C₁₋₆alkyl, —O—C₁₋₆alkyl, —[CR¹⁷R¹⁸]ₘ—O—CO—C₁₋₆alkyl, —[CR¹⁷R¹⁸]ₘ—O—C₁₋₆alkyl, —[CR¹⁷R¹⁸]ₘ—COH, and —[CR¹⁷R¹⁸]ₘ—OH; wherein
R²⁹ is hydrogen or C₁₋₆alkyl;
or R²⁹ and R²⁸ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents;
R¹⁷, R¹⁸, are each independently selected from hydrogen or C₁₋₆alkyl;
m is an integer selected from 1, 2, 3, 4 or 5; and p is an integer selected from 1 or 2.

11. The method according to claim 1, wherein the compound is of formula (IIa), 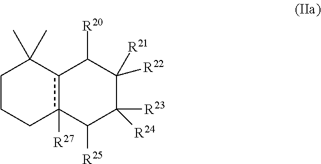
wherein the dash bond [symbol] represents an optional double bond; and wherein
R²⁰ is hydrogen or C₁₋₆alkyl;
R²¹ is hydrogen or C₁₋₆alkyl;
R²² is selected from the group comprising hydrogen, C₁₋₆alkyl, and —CO—R¹⁹;
R²³ is hydrogen or C₁₋₆alkyl;
R²⁴ is hydrogen or C₁₋₆alkyl;
or R²³ and R²⁴ together with the carbon atom to which they are attached form a 3-membered heterocyclyl moiety; wherein said heterocyclyl is optionally substituted with one or two substituents each independently selected from C₁₋₆alkyl;
R²⁵ is hydrogen or C₁₋₆alkyl;
or R²⁴ and R²⁵ form together with the carbon atom to which they are attached a 5 or 6-membered heterocyclyl moiety, wherein said heterocyclyl is optionally substituted with one or more substituents each independently selected from C₁₋₆alkyl, or wherein one of said substituent and R²³ form together with the atoms to which they are attached a 5- to 6-membered heterocyclyl moiety;
R²⁷ if present is hydrogen or C₁₋₆alkyl.

12. The method according to claim 1, wherein the compound is of formula (Ic), 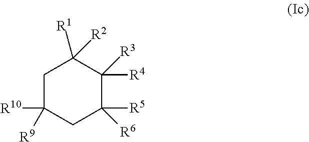
wherein R¹, R² are each independently selected from hydrogen or C₁₋₆alkyl;
R³, R⁴ are each independently selected from hydrogen or C₁₋₆alkyl;
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —CO—R¹⁹;
m is an integer selected from 1, 2, 3, 4 or 5, n is an integer selected from 0, 1, 2, 3 or 4;
X is a single bond or O,
R¹³, R¹⁴, R¹⁶, R¹⁷, R¹⁸, R¹⁹ are each independently selected from hydrogen or C₁₋₆alkyl;
R¹⁵ is OH, or R¹⁵ together with R¹⁶ form an oxo group;
or R³ and R⁵ together are of formula —O—[CR¹⁷R¹⁸]ₚ—O—, and form together with the carbon atoms to which they are attached a 5- or 6-membered heterocyclyl moiety, wherein said heterocyclyl is optionally substituted with one or more C₁₋₆alkyl substituents; p is an integer selected from 1 or 2;
R⁶ is hydrogen or C₁₋₆alkyl;
R⁹, R¹⁰ are each independently selected from hydrogen or C₁₋₆alkyl;
or R¹ with R⁹ together with the carbon atoms to which they are attached form a saturated C₅-membered ring, wherein said ring is optionally substituted with one or more C₁₋₆alkyl substituents.

13. The method according to claim 1, wherein the compound is of formula (Id), 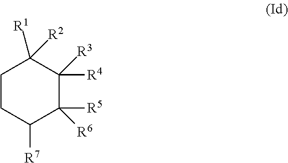
wherein R¹, R² are each independently selected from hydrogen or C₁₋₆alkyl;
R³, R⁴ are each independently selected from hydrogen or C₁₋₆alkyl;
or R¹ with R³ together with the carbon atoms to which they are attached form a saturated C3 to C5-membered ring, wherein said ring is optionally substituted with one or more C₁₋₆alkyl substituents;
R⁵ is selected from the group comprising —X—[CR¹³R¹⁴]ₙ—CR¹⁵R¹⁶—[CR¹⁷R¹⁸]ₘ—R¹⁹, —X—[CR¹³R¹⁴]ₙ—[CR¹⁷R¹⁸]ₘ—OH, —CO—R¹⁹;
m is an integer selected from 1, 2, 3, 4 or 5, n is an integer selected from 0, 1, 2, 3 or 4;
X is a single bond or O,
R¹³, R¹⁴, R¹⁶, R¹⁷, R¹⁸, R¹⁹ are each independently selected from hydrogen or C₁₋₆alkyl;
R¹⁵ is OH, or R¹⁵ together with R¹⁶ form an oxo group;
R⁶ is hydrogen or C₁₋₆alkyl;
or R⁵ and R⁶ form together ═[CR¹⁴]—[CR¹⁷R¹⁸]ₘ—OH;
or R⁵ and R⁶ together with the carbon atom to which they are attached form a 5- or 6-membered heterocyclyl moiety, wherein said heterocyclyl is optionally substituted with one or more C₁₋₆alkyl substituents;
R⁷ is hydrogen or C₁₋₆alkyl.

14. The method according to claim 1, wherein the compound is of formula (IVa), 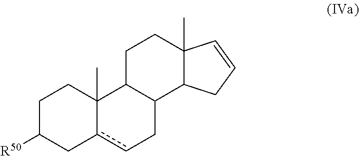
wherein the dash bond [symbol] represents an optional double bond; and wherein
R⁵⁰ is selected from OH or oxo.

15. The method according to claim 1, wherein said compound is selected from the group comprising

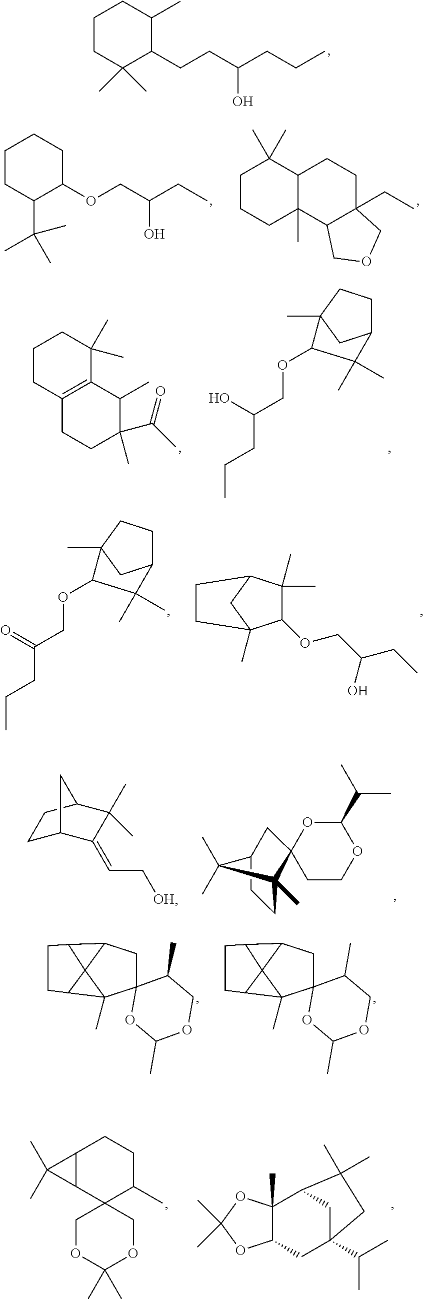

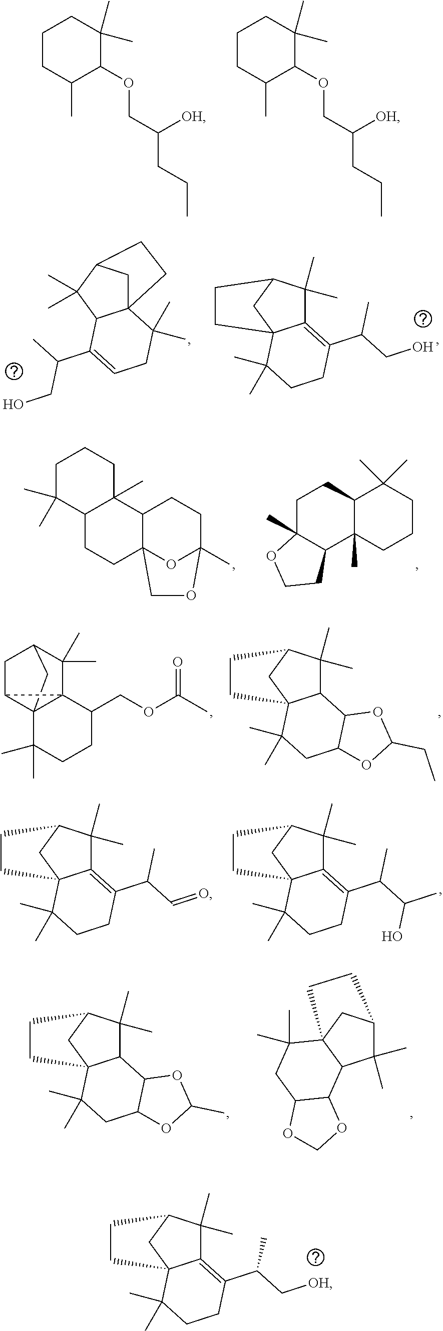

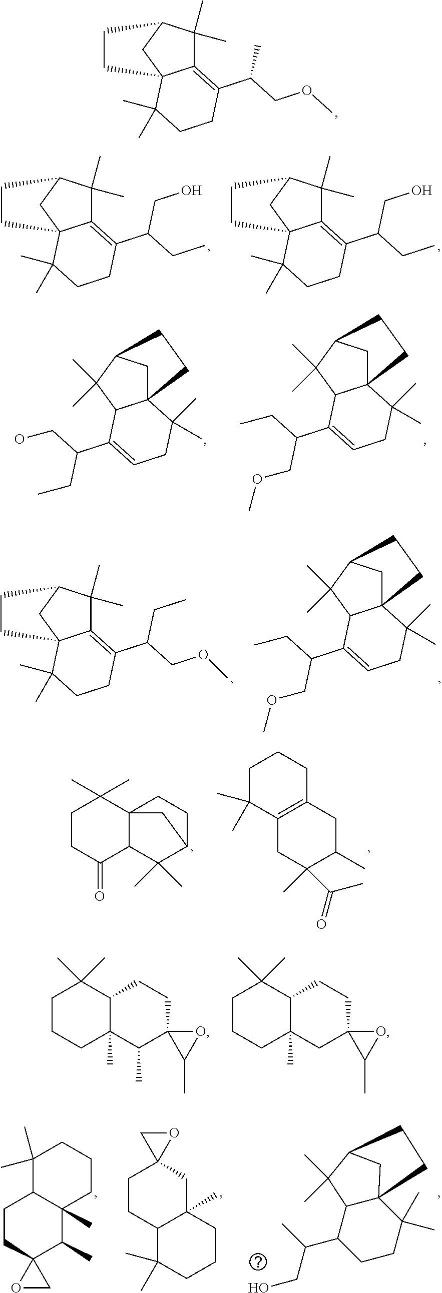

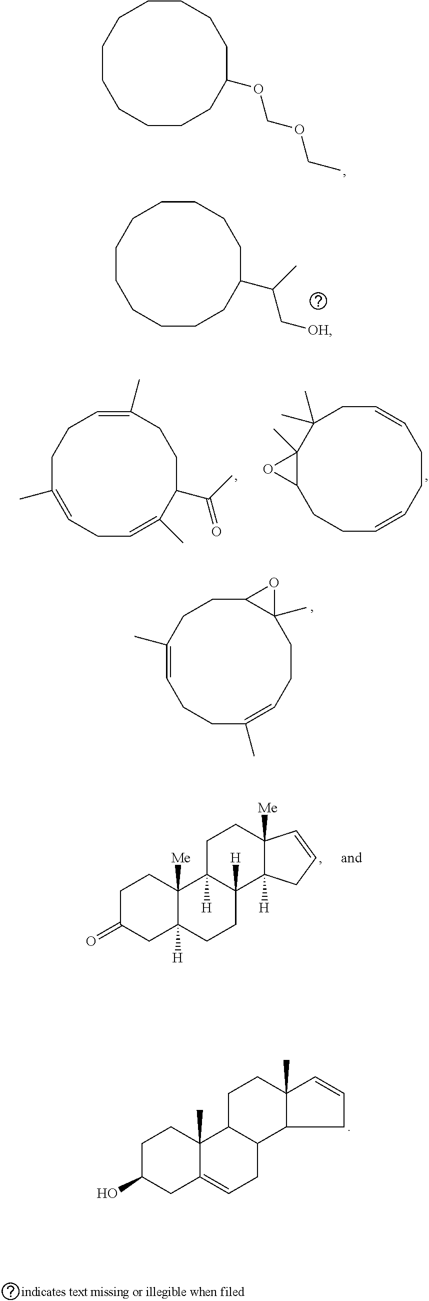

16. The method according to claim 1, wherein said compound is Amberketal or Ambermax.

17. The method according to claim 1, wherein said compound is formulated as a cosmetic composition.

18. The method according to claim 17, wherein said composition is a sunless tanning composition, a moisturizing composition or a haircare composition.

19-20. (canceled)


SEQUENCE LISTING (6 sequences)

SEQ ID NO: 1 - DNA, 20 bp, artificial sequence
  primer
cattctctttgggctgttcc

SEQ ID NO: 2 - DNA, 22 bp, artificial sequence
  primer
ttatgaaagtcctgcagtgatg

SEQ ID NO: 3 - DNA, 22 bp, artificial sequence
  primer
atggaaacaggaaatcaaacac

SEQ ID NO: 4 - DNA, 22 bp, artificial sequence
  primer
ttatgaaagtcctgcagtgatg

SEQ ID NO: 5 - DNA, 20 bp, artificial sequence
  primer
cattctctttgggctgttcc

SEQ ID NO: 6 - DNA, 20 bp, artificial sequence
  primer
gaaggaaatcacacccagga